(19) United States
(12) Patent Application Publication
(10) Pub. No.: US 2026/0355395 A1
(43) Pub. Date: Nov. 12, 2026

(54) MODULAR SINGLE-USE BREAST PUMP COMPONENTS FORMED FROM BIODEGRADABLE FIBER MATERIALS AND BARRIER MATERIALS

(71) Applicant: PUMP FOR JOY, INC., Davidson, NC (US)

(72) Inventor: Erin Devine Martin, Davidson, NC (US)

(21) Appl. No.: 19/673,759

(22) Filed: May 11, 2026

Related U.S. Application Data
(60) Provisional application No. 63/802,952, filed on May 9, 2025.
(60) Provisional application No. 63/912,919, filed on Nov. 6, 2025.

(51) Int. Cl. A61M 1/06; A61M 39/24

(52) U.S. Cl. CPC A61M 1/062; A61M 39/24

(57) ABSTRACT

Described are breast pump components comprising a first portion and a second portion configured to be coupled together to form the breast pump component. Each of the first portion and the second portion is formed from a laminate material comprising a compressed fiber material and a barrier layer disposed on at least one surface of the compressed fiber material. In an assembled configuration, the barrier layer of each of the first portion and the second portion defines at least one internal surface of the breast pump component configured for contact with lactation fluid. The compressed fiber material provides structural rigidity and reduced weight while the barrier layer provides liquid impermeability and mechanical compliance. Methods of forming breast pump components and kits comprising breast pump components are also provided.

### CROSS-REFERENCE TO RELATED APPLICATIONS

This application claims the benefit of priority to U.S. Provisional Patent Application No. 63/802,952, filed May 9, 2025 and entitled “Biodegradable Single-Use Breast Pump Components, Methods of Fabricating and Using the Same, and Kits Comprising the Same,” the entire disclosure of which is hereby incorporated herein by reference in its entirety for all purposes.

This application also claims the benefit of priority to U.S. Provisional Patent Application No. 63/912,919, filed Nov. 6, 2025 and entitled “Modular Components for Breast Pump Systems, Disposable Liners for the Same, and Methods for Assembling and Using the Same,” the entire disclosure of which is hereby incorporated herein by reference in its entirety for all purposes.

### TECHNICAL FIELD

The present disclosure relates to breast pump systems and components thereof, and more particularly to modular breast pump components formed from laminate materials comprising compressed fiber materials and barrier layers configured to provide structural rigidity, liquid impermeability, and mechanical compliance suitable for contact with lactation fluid.

### BACKGROUND

Breast pump systems are widely used by lactating individuals to express and collect breast milk for later feeding. These systems typically include a motorized or manual pump unit that generates suction, a breast shield or flange that fits over the breast to create a seal, tubing that transfers negative air pressure from the pump to the breast shield, valve assemblies that regulate milk flow, backflow protectors that prevent milk from entering the pump mechanism, and collection containers for storing expressed milk. Electronic breast pump systems may further include control panels with settings for adjusting cycle speed and suction levels, as well as power supplies such as AC adapters or battery packs for portable operation.

The components of breast pump systems that contact expressed milk during use present particular challenges for users. Breast shields, valve membranes, duckbill valves, backflow protectors, connectors, and collection containers may all come into contact with lactation fluid during pumping sessions. These milk-contacting components typically require thorough cleaning between uses to maintain hygiene and prevent bacterial growth. Because many of these components include narrow geometries, curved interiors, and integrated ducts, they can be difficult to fully access and clean. Users often rely on disassembly, washing, soaking, steaming, or boiling to ensure adequate hygiene of milk-contacting components.

The cleaning burden associated with conventional breast pump components can be particularly challenging for individuals who pump frequently or who pump away from home. Lactating individuals who work outside the home, travel, or otherwise pump in locations without convenient access to cleaning facilities may find it difficult to maintain adequate hygiene of their pump components. The time and effort associated with disassembling, cleaning, and reassembling multiple small components after each pumping session can be burdensome, especially for caregivers managing demanding schedules.

Conventional breast pump components are typically manufactured from materials such as medical-grade silicone, polypropylene, or other rigid or semi-rigid plastics. While these materials provide durability and can withstand repeated cleaning and sterilization, they are generally not biodegradable and contribute to environmental waste when disposed of at the end of their useful life. The non-biodegradable nature of these materials, combined with the cleaning requirements associated with reusable components, presents challenges for users seeking both convenience and environmental sustainability.

There remains interest in breast pump systems and components that address one or more of these considerations, including ease of use, portability, hygiene, and environmental impact.

### BRIEF SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a breast pump component is provided. The breast pump component includes a first portion of the breast pump component and a second portion of the breast pump component. The first portion and the second portion are configured to be coupled together to form the breast pump component. Each of the first portion and the second portion is formed from a laminate material comprising a compressed fiber material and a barrier layer disposed on at least one surface of the compressed fiber material. In an assembled configuration, the barrier layer of each of the first portion and the second portion defines at least one internal surface of the breast pump component that is configured for contact with lactation fluid.

According to other aspects of the present disclosure, the breast pump component may include one or more of the following features. The compressed fiber material may provide structural rigidity and reduced weight of the breast pump component and the barrier layer may provide liquid impermeability and mechanical compliance. The compressed fiber material may be biodegradable or compostable. The barrier layer may be disposed over one or more mating surfaces of the first portion and the second portion. The barrier layer may form a continuous liquid-impermeable barrier across a joint between the first portion and the second portion. The compressed fiber material may not be suitable for direct contact with lactation fluid and the barrier layer may isolate the compressed fiber material from the lactation fluid. The laminate material may resist collapse and permanent deformation when subjected to partial vacuum conditions during breast pump operation. The laminate material may elastically deform under vacuum pressure and substantially return to an original shape upon release of the vacuum. The barrier layer may reduce liquid permeation through the laminate material relative to the compressed fiber material alone. The breast pump component may comprise a breast pump flange, a valve, a duckbill valve, a flange connector, or a lactation fluid collection container. The compressed fiber material may have a thickness in a range of 0.5 mm to 3 mm and the barrier layer may have a thickness in a range of 10 micrometers to 150 micrometers, and the laminate material may resist liquid permeation such that no observable absorption of lactation fluid into the compressed fiber material occurs after exposure to lactation fluid under partial vacuum conditions representative of breast pump operation for at least one pumping session. The barrier layer may include a patterned reinforcement structure selected from lattice patterns, rib patterns, corrugations, or anisotropic reinforcement features configured to increase stiffness in selected regions of the component. The patterned reinforcement may span an interface between the first portion and the second portion to reduce deformation at a seam under vacuum loading.

According to another aspect of the present disclosure, a breast pump component is provided. The breast pump component includes a first portion and a second portion that are coupled together to form the breast pump component. Each of the first portion and the second portion is formed from a laminate material comprising a planar compressed fiber material and a barrier layer laminated onto the compressed fiber material. The barrier layers of the first portion and the second portion collectively define a continuous internal surface configured for contact with lactation fluid. The laminate material exhibits liquid impermeability and mechanical durability sufficient for use in a breast pump device under partial vacuum conditions.

According to other aspects of the present disclosure, the breast pump component may include one or more of the following features. The laminate material may resist measurable milk transport into the compressed fiber material during use. The laminate material may maintain barrier integrity during repeated vacuum cycles representative of breast pump operation. The barrier layer may have a thickness less than a thickness of the compressed fiber material. Adhesion between the barrier layer and the compressed fiber material may resist delamination during flexural deformation. The laminate material may exhibit improved flexural endurance relative to the compressed fiber material alone. The barrier layer may be selected to meet food-contact and medical-device milk-contact requirements. The laminate material may support both milk expression in a breast pump device and storage of expressed milk. The compressed fiber material may comprise one or more cellulosic fiber materials. The laminate material may be configured to withstand repeated partial-vacuum loading cycles between 80 mmHg and 280 mmHg for at least 1,000 cycles without delamination of the barrier layer from the compressed fiber material and without formation of cracks, creases, or fluid leakage paths. The compressed fiber material may have a thickness in a range of 0.5 mm to 3 mm and the barrier layer may have a thickness in a range of 10 micrometers to 150 micrometers, and the laminate material may resist liquid permeation such that no observable absorption of lactation fluid into the compressed fiber material occurs after exposure to lactation fluid under partial vacuum conditions representative of breast pump operation for at least one pumping session. The barrier layer may include a patterned reinforcement structure selected from lattice patterns, rib patterns, corrugations, or anisotropic reinforcement features configured to increase stiffness in selected regions of the component. The patterned reinforcement may span an interface between the first portion and the second portion to reduce deformation at a seam under vacuum loading.

According to another aspect of the present disclosure, a breast pump component is provided. The breast pump component includes a first portion and a second portion coupled together to form the breast pump component. Each of the first portion and the second portion is formed from a laminate material comprising a compressed cellulosic fiber material and a barrier layer laminated onto an internal surface of the compressed fiber material. The barrier layer defines all milk-contacting surfaces of the breast pump component. The laminate material exhibits both liquid impermeability sufficient to prevent milk absorption into the compressed fiber material and elastic flexure sufficient to withstand repeated partial-vacuum pumping cycles without structural failure.

According to other aspects of the present disclosure, the breast pump component may include one or more of the following features. The compressed cellulosic fiber material by itself may be permeable to lactation fluid and mechanically unsuitable for breast pump use, and laminating the barrier layer onto the compressed cellulosic fiber material may produce a composite structure that simultaneously meets liquid-impermeability requirements for milk contact and elastic flexure requirements for repeated partial-vacuum operation of a breast pump device. The compressed fiber material may have a thickness in a range of 0.5 mm to 3 mm and the barrier layer may have a thickness in a range of 10 micrometers to 150 micrometers, and the laminate material may resist liquid permeation such that no observable absorption of lactation fluid into the compressed fiber material occurs after exposure to lactation fluid under partial vacuum conditions representative of breast pump operation for at least one pumping session. The barrier layer may include a patterned reinforcement structure selected from lattice patterns, rib patterns, corrugations, or anisotropic reinforcement features configured to increase stiffness in selected regions of the component. The patterned reinforcement may span an interface between the first portion and the second portion to reduce deformation at a seam under vacuum loading.

According to another aspect of the present disclosure, a compostable breast milk storage container is provided. The compostable breast milk storage container includes two compression-molded fiber halves joined together to define an internal milk storage volume. Each half includes a polymeric biopolymer barrier coating on an inner surface thereof. The storage container has a top edge wider than a bottom edge such that the container exhibits a trapezoidal cross-section. The trapezoidal geometry is configured so that a plurality of identical containers stacked in alternating vertical orientation form a level, stable stack.

According to other aspects of the present disclosure, the compostable breast milk storage container may include one or more of the following features. The geometry may provide substantially uniform face-to-face contact between adjacent containers to improve heat transfer during freezing.

According to another aspect of the present disclosure, a compostable breast milk storage container is provided. The compostable breast milk storage container includes a rigid molded fiber body defining a sealed internal volume, a biopolymer barrier layer coating all milk-contacting surfaces, and at least one pre-formed port opening configured to accept a breast pump adapter. The port opening establishes a sealed fluid pathway from a breast pump directly into the container without an intermediate collection vessel.

According to other aspects of the present disclosure, the compostable breast milk storage container may include one or more of the following features. The port opening may be recessed relative to an outer surface of the container such that adjacent containers in an alternating-orientation stack do not mechanically interfere at the port locations.

In some embodiments, the barrier layer may be formed by coating, rather than laminating, a barrier material directly onto the compressed fiber material or other fiber-based substrate. As used herein, the term “barrier layer” encompasses any liquid-impermeable or liquid-resistant layer disposed on or within a fiber-based substrate, whether applied by lamination, coating, impregnation, dispersion application, vapor deposition, or combinations thereof. The barrier layer may comprise a coating, a film, an impregnated layer, a dispersion-applied layer, a vapor-deposited layer, or combinations thereof. Application methods for the barrier layer may include, without limitation, spray coating, dip coating, roll coating, curtain coating, slot-die coating, gravure coating, flexographic coating, rod coating, blade coating, extrusion coating, impregnation, chemical vapor deposition, physical vapor deposition, atomic layer deposition, or combinations thereof. The barrier layer may comprise one or more of bio-based polymers such as polyhydroxyalkanoates (PHA), polylactic acid (PLA), polybutylene adipate terephthalate (PBAT), polycaprolactone, or polybutylene succinate; mineral-based barriers such as clay nanocomposites, silica coatings, or calcium carbonate-filled coatings; aqueous dispersions of biodegradable polymers; cellulose-derived barriers such as nanocellulose coatings, microfibrillated cellulose coatings, or cellulose nanocrystal coatings; polysaccharide barriers such as chitosan coatings, starch-based coatings, alginate coatings, or pullulan coatings; hybrid organic/inorganic barriers combining polymeric and mineral components; fluorine-free hydrophobic systems such as alkyl ketene dimer (AKD), alkenyl succinic anhydride (ASA), natural wax coatings, or silicone-based hydrophobic treatments; protein-based barriers such as whey protein coatings, casein coatings, soy protein coatings, or zein coatings; or combinations thereof. The fiber-based substrate onto which the barrier layer is disposed may comprise one or more of paper, paperboard, molded fiber, cellulose sheet, glassine, vegetable parchment, kraft paper, cellulose-derived flexible sheet materials, compressed fiber material, densified cellulosic material, or combinations thereof. In some embodiments, the barrier layer may be applied as a single coating layer or as multiple coating layers to achieve desired barrier performance. In some embodiments, the barrier layer may penetrate partially into the fiber-based substrate to form an impregnated zone that enhances adhesion between the barrier layer and the substrate.

In some embodiments, the barrier layer may be formed by modifying the fiber-based substrate itself to render the substrate impermeable, mostly impermeable, or partially impermeable to lactation fluid. The fiber-based substrate may be soaked, saturated, or immersed in a liquid barrier material such that the barrier material penetrates into the fiber matrix and fills interstitial voids between fibers. Upon drying, curing, or solidification, the barrier material within the fiber matrix may form a continuous or semi-continuous barrier phase that prevents or reduces liquid permeation through the substrate. The liquid barrier material may comprise an aqueous dispersion of PHA, PLA, or other biodegradable polymer particles; a solution of a biodegradable polymer in a volatile solvent; a molten wax or wax emulsion; a starch-based or protein-based solution; a silicone-based or silane-based hydrophobic treatment; or combinations thereof. The soaking or immersion process may be performed at ambient temperature, at elevated temperature, under vacuum to enhance penetration, or under pressure to force the barrier material into the fiber structure.

In some embodiments, the barrier material may be embedded in, formed in, formed with, or formed within the fiber-based substrate as an integral part of the substrate rather than as a separate layer disposed on a surface thereof. The barrier material may be co-formed with the fiber-based substrate during the fiber forming process. For example, barrier polymer particles, fibers, or flakes may be blended with the cellulosic fiber furnish prior to molding, such that the barrier material is distributed throughout the fiber matrix. Upon compression, heating, or drying, the barrier polymer may melt, flow, or coalesce to form a continuous or semi-continuous barrier phase within the fiber structure. In some embodiments, the barrier material may be introduced into the fiber furnish as an aqueous dispersion of biodegradable polymer microparticles or nanoparticles that deposit onto and between the cellulosic fibers during wet forming. In some embodiments, the barrier material may comprise PHA granules, PLA particles, wax emulsion droplets, or starch granules that are incorporated into the fiber slurry before sheet formation or molding.

In some embodiments, the fiber-based substrate may be impregnated with a barrier material after forming but before final shaping of the breast pump component. Impregnation may comprise applying a liquid barrier formulation to the formed fiber substrate under conditions that cause the barrier material to penetrate into the fiber matrix. The impregnation may be performed by vacuum impregnation, pressure impregnation, capillary impregnation, or combinations thereof. The impregnated barrier material may form a gradient within the fiber substrate, with higher barrier material concentration near the milk-contacting surface and lower concentration near the outer surface. In some embodiments, the impregnated fiber substrate may be subsequently coated with an additional surface barrier layer to provide a multi-level barrier architecture combining bulk impregnation with surface coating.

In some embodiments, the barrier layer may be formed by chemical modification of the fiber-based substrate surface. The cellulosic fibers at or near the surface of the substrate may be chemically treated to render them hydrophobic or liquid-resistant. Chemical modification methods may include esterification, acetylation, silylation, grafting of hydrophobic polymer chains, crosslinking with hydrophobic agents, or treatment with reactive sizing agents such as alkyl ketene dimer (AKD) or alkenyl succinic anhydride (ASA). The chemical modification may penetrate to a controlled depth within the fiber substrate, creating a modified surface zone that resists liquid permeation while the bulk of the substrate retains its original fiber properties. In some embodiments, the chemical modification may be combined with a surface coating to provide enhanced barrier performance.

In some embodiments, the barrier layer may be formed by vapor deposition of a barrier material onto the fiber-based substrate. Chemical vapor deposition (CVD), physical vapor deposition (PVD), or atomic layer deposition (ALD) may be used to deposit a thin, conformal barrier coating onto the fiber surfaces. Vapor-deposited barrier layers may comprise silicon oxide (SiOx), aluminum oxide (AlOx), or other inorganic barrier materials that provide liquid and gas barrier properties. In some embodiments, a vapor-deposited inorganic barrier layer may be combined with an organic barrier coating to form a hybrid barrier system. The vapor deposition process may coat both the surface fibers and fibers within the near-surface region of the substrate, providing enhanced barrier performance relative to surface-only coatings.

In some embodiments, the barrier layer may comprise a combination of two or more barrier formation methods. For example, the fiber-based substrate may be first impregnated with a hydrophobic agent and then coated with a surface barrier layer. The fiber-based substrate may be first chemically modified and then laminated with a barrier film. The fiber-based substrate may include embedded barrier particles from the forming process and then receive an additional surface coating. Any combination of lamination, coating, impregnation, chemical modification, vapor deposition, and embedded barrier materials may be employed to achieve desired barrier performance for breast pump and breast milk storage applications.

In some embodiments, a breast pump flange or other breast pump component may be formed from a molded fiber substrate coated with a barrier coating. The molded fiber substrate may be compression-molded, vacuum-formed, or pressure-formed from cellulosic fibers, and the barrier coating may be applied to at least the milk-contacting surfaces of the molded fiber substrate by spray coating, dip coating, or other coating methods described herein. The barrier coating may comprise PHA, PLA, or other bio-based barrier materials. In some embodiments, a breast pump flange or other breast pump component may be formed from a molded fiber substrate laminated with a PHA film or other barrier film. The PHA film may be heat-laminated, adhesive-laminated, or extrusion-coated onto the molded fiber substrate. In some embodiments, a breastmilk storage bag may be formed from a paper-based or fiber-based flexible substrate coated with a barrier coating. The paper-based flexible substrate may comprise glassine, kraft paper, vegetable parchment, or other cellulose-derived flexible sheet material, and the barrier coating may comprise PHA, PLA, or other bio-based barrier materials applied by spray coating, dip coating, roll coating, curtain coating, slot-die coating, or other coating methods described herein. In some embodiments, a breastmilk storage bag may be formed from a paper-based or fiber-based flexible substrate laminated with a PHA film or other barrier film. The PHA film may be heat-laminated, adhesive-laminated, or extrusion-coated onto the paper-based flexible substrate. In some embodiments, a paper-based flexible pouch for breastmilk storage may be formed by folding and sealing a coated or laminated paper-based substrate, with the barrier layer facing inward to define the milk-contacting interior surface of the pouch.

According to another aspect of the present disclosure, a breast pump component is provided. The breast pump component includes a first portion and a second portion that are coupled together to form the breast pump component. Each of the first portion and the second portion is formed from a laminate material comprising a planar compressed fiber material and a barrier layer laminated onto the compressed fiber material. The barrier layers of the first portion and the second portion collectively define a continuous internal surface configured for contact with lactation fluid. The laminate material exhibits liquid impermeability and mechanical durability sufficient for use in a breast pump device under partial vacuum conditions.

According to another aspect of the present disclosure, a breast milk expression system is provided. The breast milk expression system includes a reusable breast pump, at least one disposable breast pump component formed from molded fiber and a biopolymer barrier layer, and a disposable milk storage container formed from molded fiber and a biopolymer barrier layer. The system is configured to establish a sealed milk flow path from the breast pump directly into the storage container. All milk-contacting components of the system are disposable and compostable.

According to other aspects of the present disclosure, the breast milk expression system may include one or more of the following features. The disposable breast pump component may comprise a flange and the storage container may comprise a trapezoidal, stack-stable configuration.

According to another aspect of the present disclosure, a disposable compostable breast pump flange is provided. The disposable compostable breast pump flange includes a body formed from biodegradable materials and configured to transition between a substantially planar storage configuration and a three-dimensional deployed configuration for use. The flange includes engineered fold lines and reinforcement features that cause the flange to assume the deployed configuration upon attachment to a breast pump or application of vacuum.

According to another aspect of the present disclosure, a compostable breast milk storage container is provided. The compostable breast milk storage container includes two compression-molded fiber halves joined together to define an internal milk storage volume. Each half includes a polymeric biopolymer barrier coating on an inner surface thereof. The storage container has a top edge wider than a bottom edge such that the container exhibits a trapezoidal cross-section. The trapezoidal geometry is configured so that a plurality of identical containers stacked in alternating vertical orientation form a level, stable stack.

According to other aspects of the present disclosure, the compostable breast milk storage container may include one or more of the following features. The geometry may provide substantially uniform face-to-face contact between adjacent containers to improve heat transfer during freezing.

According to another aspect of the present disclosure, a compostable breast milk storage container is provided. The compostable breast milk storage container includes a rigid molded fiber body defining a sealed internal volume, a biopolymer barrier layer coating all milk-contacting surfaces, and at least one pre-formed port opening configured to accept a breast pump adapter. The port opening establishes a sealed fluid pathway from a breast pump directly into the container without an intermediate collection vessel.

According to other aspects of the present disclosure, the compostable breast milk storage container may include one or more of the following features. The port opening may be recessed relative to an outer surface of the container such that adjacent containers in an alternating-orientation stack do not mechanically interfere at the port locations.

According to another aspect of the present disclosure, a breast milk expression system is provided. The breast milk expression system includes a reusable breast pump, at least one disposable breast pump component formed from molded fiber and a biopolymer barrier layer, and a disposable milk storage container formed from molded fiber and a biopolymer barrier layer. The system is configured to establish a sealed milk flow path from the breast pump directly into the storage container. All milk-contacting components of the system are disposable and compostable.

According to other aspects of the present disclosure, the breast milk expression system may include one or more of the following features. The disposable breast pump component may comprise a flange and the storage container may comprise a trapezoidal, stack-stable configuration.

According to another aspect of the present disclosure, a disposable compostable breast pump flange is provided. The disposable compostable breast pump flange includes a body formed from biodegradable materials and configured to transition between a substantially planar storage configuration and a three-dimensional deployed configuration for use. The flange includes engineered fold lines and reinforcement features that cause the flange to assume the deployed configuration upon attachment to a breast pump or application of vacuum.

According to another aspect of the present disclosure, a breast pump component is provided. The breast pump component includes a first portion formed from a laminate material comprising a planar compressed fiber material and a barrier layer disposed on at least one surface of the compressed fiber material, the barrier layer defining at least one inside surface of the breast pump component. The breast pump component includes a second portion formed from a laminate material comprising a planar compressed fiber material and a barrier layer disposed on at least one surface of the compressed fiber material, the barrier layer defining at least one inside surface of the breast pump component. The breast pump component includes a coupling interface by which the first portion and the second portion are joined or configured to be joined together to form the breast pump component. In an assembled configuration, the barrier layers of the first and second portions collectively define a liquid-impermeable internal surface suitable for contact with lactation fluid. The compressed fiber material provides structural support and reduced weight while the barrier layer provides regulatory compatibility and mechanical compliance.

According to another aspect of the present disclosure, a breast pump component is provided. The breast pump component includes a first portion formed from a laminate material comprising a planar compressed fiber material and a barrier layer disposed on at least one surface of the compressed fiber material, the barrier layer defining at least one inside surface of the breast pump component. The breast pump component includes a second portion formed from a laminate material comprising a planar compressed fiber material and a barrier layer disposed on at least one surface of the compressed fiber material, the barrier layer defining at least one inside surface of the breast pump component. The breast pump component includes a coupling interface by which the first portion and the second portion are joined or configured to be joined together to form the breast pump component. In an assembled configuration, the barrier layers of the first and second portions collectively define a liquid-impermeable internal surface suitable for contact with lactation fluid. The compressed fiber material provides structural support and reduced weight while the barrier layer provides regulatory compatibility and mechanical compliance.

According to other aspects of the present disclosure, the breast pump component may include one or more of the following features. The barrier layer may extend across one or more mating surfaces of the first portion and the second portion. The barrier layers of the first and second portions may be bonded together along the one or more mating surfaces. The compressed fiber material may not be suitable for direct contact with lactation fluid, and the barrier layer may isolate the lactation fluid from the compressed fiber material. The compressed fiber material may comprise a planar molded, pressed, or compressed fiber structure. The barrier layer may comprise a flexible polymer selected to permit elastic deformation of the breast pump component under vacuum conditions without forming permanent creases. The coupling interface may comprise one or more mating surfaces configured for abutment, overlap, or interengagement. The coupling interface may further comprise one or more of ridges, channels, tongues, grooves, or alignment features. The breast pump component may comprise a breast pump flange, a flange connector, a valve, a duckbill valve, a backflow preventer, or a lactation fluid collection container. The component may be configured to be biodegradable or compostable after use. Neither the first portion nor the second portion may be injection molded as a unitary plastic body. The compressed fiber material may define a majority of the structural thickness of the component and the barrier layer may define a minority thickness of the component. Structural rigidity of the component may be provided primarily by the compressed fiber material rather than by the barrier layer. The barrier layer may have a thickness insufficient to maintain the structural shape of the component in the absence of the compressed fiber material. The compressed fiber material may not be approved for direct contact with lactation fluid, and the barrier layer may provide a regulatory barrier between the compressed fiber material and the lactation fluid. The barrier layer may be selected to satisfy food-contact or medical-device fluid-contact requirements independently of the compressed fiber material. The compressed fiber material may be selected without regard to food-contact regulatory approval and the barrier layer may compensate for such regulatory limitations. The first portion and the second portion may be configured to be shipped in an unassembled state and joined together by a user prior to first use. Assembly of the first and second portions by a user may enable formation of an internal fluid-contact surface that does not exist prior to assembly. The internal fluid-contact surface may be completed upon joining of the barrier layers of the first and second portions. The barrier layer may be configured to elastically deform under partial vacuum while the compressed fiber material resists permanent deformation. The laminate material may be configured to bend or flex under vacuum loading without forming creases or stress concentrations within the compressed fiber material. The barrier layer may distribute vacuum-induced stress across the compressed fiber material to prevent localized failure points. The barrier layer of the first portion may be bonded directly to the barrier layer of the second portion to form a sealed joint. The compressed fiber materials of the first and second portions may not be directly bonded to one another at a fluid-contact interface. The barrier layer may extend continuously across a joint between the first portion and the second portion. At least one portion of the component may have a mass lower than an equivalent injection-molded plastic component of the same geometry. The compressed fiber material may provide a reduction in shipping weight relative to an injection-molded plastic breast pump component providing equivalent structural strength. The component may be configured to be compostable after use while maintaining regulatory compliance during use. The component may comprise a breast pump flange having a concave geometry formed primarily by the compressed fiber material and lined by the barrier layer. The component may comprise a valve or duckbill valve, and the barrier layer may define a flexible fluid seal while the compressed fiber material supports external geometry. The barrier layer may reduce liquid permeation through the laminate material by at least an order of magnitude relative to the compressed fiber material alone. The laminate material may exhibit no detectable bulk transport of lactation fluid through the compressed fiber material during use of the breast pump component. The laminate material may exhibit a fluid transmission rate sufficiently low to prevent wicking, absorption, or diffusion of lactation fluid into the compressed fiber material during normal pumping and storage conditions. The barrier layer may form a continuous impermeable barrier across regions of the component subjected to vacuum-induced pressure differentials. The laminate material may maintain liquid impermeability after repeated vacuum cycles experienced during breast pumping. The laminate material may demonstrate lower milk permeation, absorption, or retention than an uncoated compressed fiber structure of comparable geometry. The laminate material may exhibit greater resistance to mechanical collapse under partial vacuum than the compressed fiber material alone. The barrier layer may contribute to load sharing across the compressed fiber material during use of the breast pump component. The laminate material may resist cracking, delamination, or structural failure when subjected to vacuum pressures generated during use of a breast pump device. The laminate material may maintain structural integrity under repeated cyclic loading and unloading associated with breast pump operation. The barrier layer may mitigate stress concentrations within the compressed fiber material. The laminate material may exhibit improved tensile strength or flexural strength relative to the compressed fiber material alone. The laminate material may elastically deform under partial vacuum and substantially return to its original shape upon release of the vacuum. The barrier layer may enable controlled flexure of the laminate material without permanent creasing of the compressed fiber material. The laminate material may exhibit reduced hysteresis during repeated vacuum cycles relative to the compressed fiber material alone. The barrier layer may allow limited elastic stretching while the compressed fiber material constrains overall deformation. The laminate material may exhibit fatigue resistance sufficient to withstand a plurality of pumping cycles without loss of barrier or mechanical function. The laminate material may maintain impermeability and mechanical integrity after a number of pumping cycles typical of daily use of a breast pump device. The laminate material may resist pinhole formation, microcracking, or delamination after repeated flexural deformation. The barrier layer may protect the compressed fiber material from moisture-induced degradation during use. The laminate material may exhibit greater durability under wet conditions than the compressed fiber material alone. The laminate material may demonstrate a performance characteristic selected from impermeability, flexural durability, or vacuum resistance that would not be expected based on the properties of the compressed fiber material. The laminate material may exhibit a synergistic improvement in mechanical and barrier properties relative to the compressed fiber material and the barrier layer considered individually. The barrier layer may enable use of a compressed fiber material that would otherwise be unsuitable for breast pump applications. The laminate material may satisfy both fluid-contact performance requirements and mechanical performance requirements that the compressed fiber material alone cannot satisfy. The laminate material may exhibit a measured liquid permeability, flexural fatigue endurance, or vacuum deformation characteristic demonstrating suitability for breast pump use. Testing may demonstrate that the laminate material resists fluid penetration while maintaining mechanical compliance under simulated breast pump operating conditions. Measured performance of the laminate material under vacuum loading may differ qualitatively from the performance of the compressed fiber material alone. The laminate material may resist structural collapse under a partial vacuum of at least 150 mmHg, at least 200 mmHg, or at least 250 mmHg without permanent deformation. The laminate material may maintain dimensional integrity when subjected to repeated vacuum cycles within a range of 50 mmHg to 300 mmHg. The laminate material may exhibit a maximum permanent deformation of less than 5%, less than 3%, or less than 1% after vacuum loading representative of breast pump operation. The laminate material may have a flexural modulus in a range of 0.1 to 5 GPa, 0.3 to 3 GPa, or 0.5 to 2 GPa, measured in bending. The barrier layer may increase flexural endurance of the laminate material by at least 25%, at least 50%, or at least 100% relative to the compressed fiber material alone. The laminate material may withstand at least 500, at least 1,000, or at least 5,000 flexural deformation cycles without cracking or delaminating. The laminate material may exhibit an elastic strain at break in a range of 1% to 20%, 2% to 15%, or 5% to 10%. The barrier layer may exhibit greater elongation at break than the compressed fiber material and accommodate elastic deformation during vacuum cycling. The laminate material may exhibit fatigue resistance sufficient to withstand at least 2,000 vacuum cycles without loss of barrier or mechanical function. The laminate material may exhibit a liquid permeation rate of less than 1 g/m²/day, less than 0.5 g/m²/day, or less than 0.1 g/m²/day. The laminate material may reduce liquid absorption into the compressed fiber material by at least 90%, at least 95%, or at least 99% relative to uncoated fiber material. Transport of lactation fluid through the laminate material may be substantially prevented over a continuous exposure period of at least 4 hours, at least 12 hours, or at least 24 hours. The barrier layer may exhibit a water vapor transmission rate (WVTR) of less than 20 g/m²/day, less than 10 g/m²/day, or less than 5 g/m²/day. The compressed fiber material may remain substantially dry after repeated exposure to lactation fluid due to the barrier properties of the barrier layer. Adhesion between the barrier layer and the compressed fiber material may exhibit a peel strength of at least 1 N/cm, at least 3 N/cm, or at least 5 N/cm. The laminate material may resist delamination under cyclic mechanical loading representative of breast pump use. Bonding between the barrier layer and the compressed fiber material may be maintained after exposure to moisture, vacuum, and mechanical deformation. The compressed fiber material may comprise one or more of: cellulose fibers, wood pulp fibers, bamboo fibers, bagasse fibers, hemp fibers, flax fibers, cotton fibers, recycled paper fibers. The compressed fiber material may comprise a blend of two or more fibrous materials selected to balance stiffness and toughness. The compressed fiber material may comprise at least 50 wt%, at least 70 wt%, or at least 90 wt% cellulosic fibers. The compressed fiber material may have a density in a range of 0.3 to 1.2 g/cm³, 0.4 to 0.8 g/cm³, or 0.5 to 0.7 g/cm³. The compressed fiber material may have a thickness in a range of 0.3 mm to 5 mm, 0.5 mm to 3 mm, or 1 mm to 2 mm. The barrier layer may comprise one or more of: polyethylene, polypropylene, ethylene-vinyl acetate (EVA), polyurethane, polycaprolactone, polylactic acid, polyhydroxyalkanoates (PHA), polybutylene adipate terephthalate (PBAT). The barrier layer may comprise a multilayer film including at least one elastomeric layer and at least one barrier layer. The barrier layer may have a thickness in a range of 5 μm to 300 μm, 10 μm to 150 μm, or 25 μm to 100 μm. The barrier layer may have an elongation at break of at least 100%, at least 200%, or at least 400%. The barrier layer may exhibit a Shore hardness selected to permit elastic deformation under vacuum loading. The laminate material may exhibit mechanical and barrier performance that exceeds the sum of the individual contributions of the compressed fiber material and the barrier layer when considered separately. Experimental testing may demonstrate that the laminate material enables use of compressed fiber material in a breast pump application that would otherwise be mechanically or hygienically unsuitable. The laminate structured material may exhibit unexpected resistance to both mechanical failure and fluid permeation relative to conventional breast pump component materials. The barrier layer may be configured to satisfy biocompatibility requirements for materials contacting lactation fluid in a medical device pursuant to a biological evaluation conducted in accordance with ISO 10993-1. Materials defining milk-contacting surfaces may exhibit no cytotoxic response when evaluated using an in-vitro cytotoxicity assay consistent with ISO 10993-5. Materials defining milk-contacting surfaces may exhibit no irritation or sensitization response when evaluated under conditions consistent with ISO 10993-10. Chemical characterization testing may demonstrate that extractable or leachable substances from the barrier layer remain below toxicological concern thresholds established under FDA biocompatibility guidance. The barrier layer may be selected as a food-contact substance authorized for contact with human milk under 21 CFR Parts 175, 176, or 177. The laminate material may exhibit overall migration values below 10 mg/dm² when tested under food-contact conditions representative of human milk storage. Specific migration of constituent substances from the barrier layer into human milk may remain below FDA-recognized thresholds of toxicological concern. The laminate material may maintain food-contact compliance under conditions of refrigerated storage and frozen storage of human milk. The barrier layer may prevent direct contact between lactation fluid and the compressed fiber material to satisfy FDA food-contact safety requirements. Measured liquid permeation through the laminate material may be sufficiently low to prevent fiber absorption that would otherwise disqualify the compressed fiber material from food-contact use. The laminate architecture may enable use of a compressed fiber substrate that is not independently approved for food contact by providing a compliant polymeric barrier. The laminate material may maintain mechanical integrity under vacuum levels representative of FDA-cleared breast pump operation. Cyclic vacuum testing may demonstrate no cracking, delamination, or barrier failure after a number of cycles representative of normal breast pump use. Mechanical testing may demonstrate suitability of the laminate material for repeated use in a medical device subject to partial vacuum loading. The barrier layer may resist chemical degradation when exposed to human milk under repeated use conditions. The laminate material may maintain barrier and mechanical properties following exposure to moisture, temperature cycling, and vacuum loading consistent with FDA device performance expectations. The same laminate material architecture may support compliance with both FDA medical-device requirements for milk expression and FDA food-contact requirements for milk storage. Testing may demonstrate that the laminate material meets regulatory requirements applicable to both breast pump components and breastmilk storage containers. The laminate material may be configured to be suitable for use in both a Class II breast pump device and a food-contact article for human milk. Experimental testing may demonstrate that the laminate structured material simultaneously satisfies FDA milk-contact safety requirements and mechanical durability requirements that the compressed fiber material alone cannot satisfy. Achieving FDA compliance for milk contact using the laminated compressed fiber material may not have been expected based on the properties of the compressed fiber material.

According to another aspect of the present disclosure, a method of forming a breast pump component is provided. The method includes forming a first portion of the breast pump component from a laminate material comprising a planar compressed fiber material and a barrier layer disposed on at least one surface of the compressed fiber material. The method includes forming a second portion of the breast pump component from a laminate material comprising a planar compressed fiber material and a barrier layer disposed on at least one surface of the compressed fiber material. The method includes configuring the first portion and the second portion such that the barrier layer of each portion defines at least one inside surface configured for contact with lactation fluid when the breast pump component is assembled. The method includes joining or coupling the first portion and the second portion together to form the breast pump component. The compressed fiber material provides structural rigidity, reduced weight, and biodegradability. The barrier layer provides a liquid-impermeable, food-contact-compatible, and mechanically compliant surface suitable for use in a breast pump device.

According to other aspects of the present disclosure, the method may include one or more of the following features. Forming the laminate material may comprise laminating the barrier layer onto the compressed fiber material using heat, pressure, adhesives, extrusion coating, or combinations thereof. The barrier layer may be disposed on surfaces intended to define an internal fluid pathway of the breast pump component. The barrier layer may be disposed across one or more mating surfaces of the first portion and the second portion. Joining or coupling the first portion and the second portion together may comprise thermally bonding the barrier layers of the first and second portions to one another. Joining or coupling the first portion and the second portion may comprise using adhesives, ultrasonic welding, mechanical fasteners, elastic fasteners, interference fits, snap-fit features, or combinations thereof. The first portion and the second portion may be joined together during manufacture to form a unitary breast pump component prior to distribution to a user. The first portion and the second portion may be provided separately to a user and joined together by the user prior to use. The compressed fiber material may comprise a biodegradable and compostable material. The barrier layer may be selected to be compliant such that the assembled breast pump component flexes under partial vacuum conditions during pumping without creasing, cracking, or forming localized failure regions. Forming the laminate material may result in a breast pump component that simultaneously exhibits liquid impermeability and elastic flexure during use. Bonding the barrier layer to the compressed fiber material may increase resistance to fluid permeation and mechanical failure relative to an unbonded structure.

According to another aspect of the present disclosure, a method of manufacturing a breast pump component is provided. The method includes forming a bio-based structural substrate, forming a barrier, applying the barrier to at least one surface of the bio-based structural substrate to form a laminate structure, and forming the laminate structure into a breast pump component having one or more milk-contacting surfaces defined by the barrier.

According to other aspects of the present disclosure, the method may include one or more of the following features. Forming the barrier may comprise forming the barrier by extrusion. Forming the barrier may comprise forming the barrier by blown-film extrusion. Forming the barrier may comprise forming the barrier by cast-film extrusion. Forming the barrier may comprise forming the barrier by solution casting. Forming the barrier may comprise forming the barrier by melt casting. Forming the barrier may comprise forming the barrier by calendaring. Forming the barrier may comprise forming the barrier by extrusion coating. Forming the barrier may comprise forming the barrier as a multilayer or co-extruded film. Forming the barrier may comprise forming the barrier from a biodegradable polymer selected from polyhydroxyalkanoates, polylactic acid, polybutylene adipate terephthalate, polycaprolactone, or combinations thereof. Applying the barrier to the bio-based structural substrate may comprise laminating the barrier to the substrate using heat and pressure. Applying the barrier to the bio-based structural substrate may comprise adhesive lamination. Applying the barrier to the bio-based structural substrate may comprise extrusion coating the barrier directly onto the substrate. Applying the barrier to the bio-based structural substrate may comprise spray coating the barrier or a polymeric precursor onto the substrate. Applying the barrier to the bio-based structural substrate may comprise dip coating the substrate in a polymeric dispersion or solution. Applying the barrier to the bio-based structural substrate may comprise roll-to-roll coating or roll-to-roll lamination. Applying the barrier to the bio-based structural substrate may comprise applying the barrier only to surfaces intended to contact lactation fluid. Forming the bio-based structural substrate may comprise forming a compressed fiber material. Forming the compressed fiber material may comprise wet molded fiber forming. Forming the compressed fiber material may comprise dry molded fiber forming. Forming the compressed fiber material may comprise thermoforming, compression molding, or hot pressing of fibrous material. The compressed fiber material may comprise one or more of cellulose fibers, bagasse fibers, bamboo fibers, wood pulp fibers, recycled paper fibers, wheat straw fibers, hemp fibers, or combinations thereof. Forming the compressed fiber material may comprise blending multiple fibrous materials prior to molding to tailor mechanical properties. Forming the compressed fiber material may comprise adding a binder selected from starch-based binders, waxes, alkyl ketene dimer, or biodegradable resins. The method may further comprise forming the breast pump component in at least two separate portions. The method may further comprise joining the at least two separate portions together to form the breast pump component. Joining the at least two separate portions may comprise heat sealing opposing barrier surfaces to one another. Joining the at least two separate portions may comprise ultrasonic welding, adhesive bonding, mechanical fastening, or combinations thereof. Joining the at least two separate portions may form a liquid-impermeable seam without use of a separate gasket or sealing member. The method may further comprise patterning the barrier prior to or after application to the substrate to locally modify mechanical properties of the laminate structure. Patterning the barrier may comprise laser cutting, embossing, scoring, corrugating, or forming lattice or rib structures. The method may further comprise forming the laminate structure into a breast pump flange, valve housing, connector, diaphragm support, or milk storage container. The method may further comprise sterilizing the breast pump component after manufacture. Sterilizing may comprise gamma irradiation, electron-beam irradiation, ethylene oxide exposure, or combinations thereof. The method may produce a breast pump component that is biodegradable or compostable after use. The laminate structure may exhibit liquid impermeability sufficient to prevent absorption of lactation fluid into the bio-based structural substrate during use. The laminate structure may exhibit mechanical durability sufficient to withstand repeated partial-vacuum pumping cycles without delamination or structural failure.

According to another aspect of the present disclosure, a breast pump component architecture is provided. The breast pump component architecture includes a structural layer comprising a compressed fiber material configured to provide rigidity, shape retention, and reduced mass of a breast pump component. The breast pump component architecture includes a fluid-contact layer comprising a barrier disposed on the structural layer, the barrier being liquid-impermeable, food-contact-compatible, and mechanically compliant. The breast pump component architecture is configured such that the compressed fiber material supplies structural support while the barrier supplies lactation fluid compatibility and flexural performance during use of a breast pump device.

According to other aspects of the present disclosure, the breast pump component architecture may include one or more of the following features. The barrier layer may be bonded to the compressed fiber material across a major planar surface. The compressed fiber material may comprise a compostable fiber-based substrate. The barrier layer may prevent absorption of lactation fluid into the compressed fiber material. The barrier layer may be configured to maintain integrity under repeated vacuum cycles during breast pumping. The breast pump component architecture may be implemented in a multi-part breast pump component comprising at least two laminate portions joined together. The barrier layer may span across mating surfaces of the at least two laminate portions to form a continuous internal fluid pathway.

According to another aspect of the present disclosure, a flexible compostable breast milk storage bag is provided. The flexible compostable breast milk storage bag includes a flexible cellulosic substrate and a bio-based barrier layer disposed on at least one interior surface of the flexible cellulosic substrate. The flexible cellulosic substrate and the bio-based barrier layer together define a sealed or sealable internal volume configured to store expressed human breast milk. The bio-based barrier layer provides liquid impermeability sufficient to contain expressed breast milk under refrigerated and frozen storage conditions. The flexible compostable breast milk storage bag is compostable without separation of constituent materials.

According to other aspects of the present disclosure, the flexible compostable breast milk storage bag may include one or more of the following features. The flexible cellulosic substrate may comprise glassine paper. The bio-based barrier layer may comprise a plant-derived, heat-sealable barrier coating derived from renewable agricultural feedstocks. The bio-based barrier layer may comprise one or more of polyhydroxyalkanoates (PHA), polylactic acid (PLA), or other bio-based barrier materials. The flexible bag may be plastic-free and PFAS-free. The flexible bag may be industrially compostable, home compostable, or both. The bag may comprise an open-top configuration with a gusseted base configured to provide stand-up capability when the bag is filled with expressed milk. The bag may comprise a three-side sealed construction with an open top configured for consumer filling and sealing. The bag may include a tear notch at a sealed top edge configured for easy consumer opening after sealing. Volume markings may be visible through the translucent substrate to permit measurement of milk volume without opening the bag. The flexible bag may be configured for direct pump-to-bag filling. The bag may include an opening or port configured to receive a breast pump adapter or to interface directly with a duckbill valve or other component of a breast pump device, establishing a sealed fluid pathway from the breast pump into the bag without an intermediate collection vessel.

According to another aspect of the present disclosure, a breast milk expression and storage system is provided. The system includes a reusable breast pump, at least one disposable breast pump component formed from a laminate material comprising a compressed fiber material and a barrier layer, and a flexible compostable breast milk storage bag comprising a flexible cellulosic substrate and a bio-based barrier layer disposed on at least one interior surface thereof. The system is configured to establish a sealed milk flow path from the breast pump through the disposable breast pump component and directly into the flexible compostable breast milk storage bag. All milk-contacting components of the system are disposable and compostable.

According to other aspects of the present disclosure, the breast milk expression and storage system may include one or more of the following features. The system may further comprise a bag-direct pumping adapter configured to establish a sealed fluid pathway between the disposable breast pump component and the flexible compostable breast milk storage bag. The system may further comprise a dedicated consumer heat sealer configured to seal the flexible compostable breast milk storage bag after filling. The interior of the bag may be sanitized or sterilized prior to final sealing using non-thermal methods including UV-C irradiation, electron-beam radiation, gamma radiation, or other ionizing or non-ionizing treatment methods.

According to another aspect of the present disclosure, a kit is provided. The kit comprises a breast pump component as described above.

According to another aspect of the present disclosure, an apparatus is provided. The apparatus comprises a breast pump component as described above.

According to another aspect of the present disclosure, an apparatus is provided. The apparatus comprises at least one processor and at least one memory comprising instructions stored therein that, when executed by the at least one processor, cause the apparatus to perform a method of forming a breast pump component as described above.

The foregoing general description of the illustrative embodiments and the following detailed description thereof are merely exemplary aspects of the teachings of this disclosure and are not restrictive.

### BRIEF DESCRIPTION OF FIGURES

Having thus described the invention in general terms, reference will now be made to the accompanying drawings. The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1 is a block diagram illustrating an example breast pumping system, according to an embodiment of the present disclosure.

FIG. 2 is a schematic illustration of an example breast pumping system, according to an embodiment of the present disclosure.

FIG. 3 is a schematic illustration from a perspective view of assembled breast pumping components, according to an embodiment of the present disclosure.

FIG. 4 is a schematic illustration from a perspective view of disassembled breast pumping components provided or received as a kit, according to an embodiment of the present disclosure.

FIGS. 5A-5C are schematic illustrations of a breast shield, according to one or more embodiments of the present disclosure.

FIGS. 6A-6B are schematic illustrations of a breast shield connector, according to one or more embodiments of the present disclosure.

FIGS. 7A-7B are schematic illustrations of a duckbill connector, according to one or more embodiments of the present disclosure.

FIGS. 8A-8K are schematic illustrations of a duckbill valve, according to one or more embodiments of the present disclosure.

FIG. 9 is a top exploded view of a modular breast pump flange, according to one or more embodiments of the present disclosure.

FIG. 10 illustrates side views of two halves of a modular breast pump flange, according to one or more embodiments of the present disclosure.

FIG. 11 illustrates side views of two halves of a modular breast pump flange, according to one or more embodiments of the present disclosure.

FIG. 12 illustrates side views of two halves of a modular breast pump flange, according to one or more embodiments of the present disclosure.

FIGS. 13A and 13B are side view illustration of two halves of a modular breast pump flange, according to one or more embodiments of the present disclosure.

FIG. 14 is a side view of a breast pump flange and a liner configured to be disposed within an inner volume of the breast pump flange, according to one or more embodiments of the present disclosure.

FIG. 15 is a side view of a breast pump flange and a liner configured to be disposed within an inner volume of the breast pump flange, according to one or more embodiments of the present disclosure.

FIG. 16 is a side view of a breast pump flange and a disposable liner configured to be disposed within an inner volume of the breast pump flange, according to one or more embodiments of the present disclosure.

FIG. 17 is a side view of a breast pump flange and a liner configured to be disposed within an inner volume of the breast pump flange, according to one or more embodiments of the present disclosure.

FIG. 18 is a side view of a breast pump flange and a disposable liner configured to be disposed within an inner volume of the breast pump flange, according to one or more embodiments of the present disclosure.

FIG. 19 is a top view of an unassembled disposable liner configured to be disposed within an inner volume of a breast pump component, according to one or more embodiments of the present disclosure.

FIG. 20 is a side view illustration of two unassembled halves of a breast pump component, according to one or more embodiments of the present disclosure.

FIG. 21 is a perspective view of the breast pump component assembled from two component halves, according to one or more embodiments of the present disclosure.

FIG. 22 is a side view illustration of two halves of a breast pump component, according to one or more embodiments of the present disclosure.

FIG. 23 is a perspective view of a breast pump component assembled from two halves of the component, according to one or more embodiments of the present disclosure.

FIG. 24 illustrates side views of a breast pump component and a disposable liner for the breast pump component, according to one or more embodiments of the present disclosure.

FIG. 25 illustrates side views of a half of a breast pump component and a disposable liner configured to be disposed within an inner volume of the half of the breast pump component, according to one or more embodiments of the present disclosure.

FIG. 26 is a side view of a lactation fluid collection container, according to one or more embodiments of the present disclosure.

FIG. 27 is a side view of a lactation fluid collection container, according to one or more embodiments of the present disclosure.

FIG. 28 is a side view of a lactation fluid collection container, according to one or more embodiments of the present disclosure.

FIG. 29A is a perspective view of unassembled halves of a modular breast pump flange, according to one or more embodiments of the present disclosure.

FIG. 29B is a perspective view of assembled halves of a modular breast pump flange, according to one or more embodiments of the present disclosure.

FIG. 30 is a perspective view of unassembled halves of a modular breast pump flange in a nested configuration, according to one or more embodiments of the present disclosure.

FIG. 31 is a block flow diagram illustrating an example method for assembling and/or using a breast pump component, according to one or more embodiments of the present disclosure.

FIG. 32 is a block flow diagram illustrating an example method for assembling and/or using a breast pump component, according to one or more embodiments of the present disclosure.

FIG. 33 is a block flow diagram illustrating an example method for assembling and/or using a breast pump component, according to one or more embodiments of the present disclosure.

FIG. 34 is a block flow diagram illustrating an example method for assembling and/or using a breast pump component, according to one or more embodiments of the present disclosure.

FIG. 35 is a block flow diagram illustrating an example method for assembling and/or using a breast pump component, according to one or more embodiments of the present disclosure.

FIG. 36 is a block flow diagram illustrating an example method for assembling and/or using a breast pump component, according to one or more embodiments of the present disclosure.

FIG. 37 is a block flow diagram illustrating an example method for assembling and/or using a breast pump component, according to one or more embodiments of the present disclosure.

FIG. 38 is a side profile view of a planar laminate material comprising a compressed fiber material and a barrier layer, according to one or more embodiments of the present disclosure.

FIG. 39 is a perspective view of a breast shield formed from a laminate material, according to one or more embodiments of the present disclosure.

FIG. 40 is an exploded perspective view of a modular breast shield formed from a laminate material, according to one or more embodiments of the present disclosure.

FIG. 41 is an exploded perspective view of a modular breast shield formed from a laminate material with compressed fiber material comprising the full outer surface, according to one or more embodiments of the present disclosure.

FIG. 42 is an exploded perspective view of a modular breast shield formed from a laminate material with a barrier layer wrapping over skin-contacting and coupling edges, according to one or more embodiments of the present disclosure.

FIG. 43 is a side perspective view of assembled components for a breast pump device, according to one or more embodiments of the present disclosure.

FIG. 44 is a perspective view schematic of molds for forming two halves of a breast pump component for a breast pump device, according to one or more embodiments of the present disclosure.

FIG. 45 is an image of molds for forming two halves of a breast pump component for a breast pump device, according to one or more embodiments of the present disclosure.

FIG. 46 is a block flow diagram of a method for forming a breast pump component for a breast pump device, according to one or more embodiments of the present disclosure.

FIG. 47 is a block flow diagram of a method for forming a breast pump component for a breast pump device, according to one or more embodiments of the present disclosure.

FIG. 48 is a simplified side-view schematic of a process for forming a breast pump component for a breast pump device, according to one or more embodiments of the present disclosure.

FIG. 49 is a cross-sectional side view of a bio-based material comprising a barrier-coating material, according to one or more embodiments of the present disclosure.

FIG. 50 is a top-down cross-sectional view of a lactation fluid storage pouch formed from a bio-based material comprising a barrier-coating, according to one or more embodiments of the present disclosure.

### DETAILED DESCRIPTION

The present disclosure more fully describes various embodiments with reference to the accompanying drawings. It should be understood that some, but not all embodiments are shown and described herein. Indeed, the embodiments may take many different forms, and accordingly this disclosure should not be construed as limited to the embodiments set forth herein. Rather, these embodiments are provided so that this disclosure will satisfy applicable legal requirements. Like numbers refer to like elements throughout.

Various embodiments of the present disclosure will now be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level. Like numbers refer to like elements throughout.

Thus, the inventor has conceived of and diligently reduced to practice multiple embodiments of a system, method of manufacture, method of use, composition of matter, apparatus, device, and/or a kit for facilitating breast pumping. In some embodiments, various components of a breast pump system, device, assembly, or kit can be or comprise biodegradable material(s).

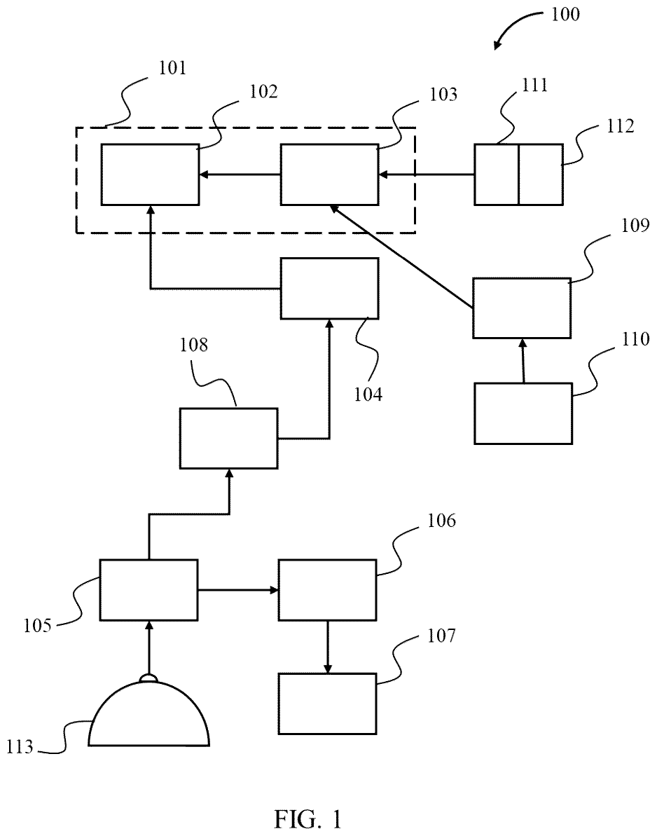

FIG. 1 is a block diagram of an example of a breast pump system 100 that comprises components that work together to facilitate human milk expression efficiently and comfortably. The breast pump system 100 can include a housing 101 that at least partially houses therein a pump 102 operably coupled to a motor 103 that together generate suction to draw milk from milk ducts in a breast 113 of a lactating person. Connected to the motor 103 is tubing 104, which transfers a negative air pressure to an inner volume within a breast shield 105. The breast shield 105, also known as a flange, is a molded plastic or silicone piece designed to fit over a nipple and surrounding breast tissue of a human breast to create a seal therebetween. Attached to the breast shield 105 is a valve membrane system 106, which facilitates controlled suction and milk flow from the breast 113, through the breast shield 105, and into a collection container 107. The collection container 107 serves as a milk reservoir and is typically made of BPA-free plastic or glass. In some embodiments, the breast pump system 100 also includes an optional backflow protector 108, also known as a backflow preventer, to prevent milk from entering the tubing 104, ensuring hygiene within components of the breast pump system 100 that are more difficult or impossible to clean and to improve the lifespan of the motor 103 and pump 102. Additionally, an adjustable suction control unit 109 allows the lactating person to modify the suction strength for comfort and efficiency. Many modern breast pumps feature an electronic control panel 110 with settings for cycle speed and suction levels.

The pump 103 is caused to operate by operation of the motor 102. In some embodiments, the motor 102 of the breast pump system 100 can be powered by, e.g., an AC adapter 111 for stationary use or a battery pack 112 for portability.

The breast pump system 100 is a complex yet highly efficient device designed to mimic an infant's natural nursing patterns through controlled vacuum cycles. The housing 101 in which the pump 102 and/or the motor 103 is typically housed, can be or comprise a compact plastic or metal casing with internal diaphragms and pistons to generate intermittent suction pressure. The motor 102 can be mechanically, magnetically, hydraulically, pneumatically, and/or electrically driven. The motor 102 can, for example, be powered via either the AC adapter 111 or the battery pack 112. The suction generated by the pump 103 can be transmitted through the tubing 104, which is generally made of medical-grade silicone or flexible thermoplastic elastomers. The tubing 104 typically has a standardized inner diameter that ensures airtight connections to the pump 103 and one or more other components, such as the breast shield 105 or another component fluidically coupled thereto.

The breast shield 105 can be made from a transparent or a semi-transparent BPA-free polymeric material, such as polypropylene, polylactic acid, or the like. The breast shield 105 can comprise a funnel-shaped component designed to fit securely over the breast. In some embodiments, the breast shield 105 comprises a wide mouth and a tapered neck to accommodate various nipple sizes and/or breast sizes and ensure an ergonomic seal between the outside of the breast 113 and the inside of the breast shield 105. In some embodiments, the breast shield 105 can be dimensioned and configured to connect directly to the valve membrane system 106.

In some embodiments, the valve membrane system 106 can comprise a flexible silicone or rubber membrane that acts as a one-way valve, preventing backflow and regulating milk extraction efficiency. Below the valve assembly, the collection container 107 is securely threaded or press-fit onto the breast shield 105 or otherwise fluidically coupled thereto. In some embodiments, the collection container 107 is dimensioned and configured to accommodate volumes of expressed milk and/or other expressed fluids (e.g., colostrum) ranging from about 4 fluid ounces (about 118 mL) to about 8 fluid ounces (about 236 mL). The collection container 107 can comprise, or be made of, food-grade polypropylene or borosilicate glass to ensure durability and ease of sterilization. In some embodiments, the collection container 107 is a food-grade polymeric bag, such as a sealable storage bag into which the expressed fluids (e.g., milk, colostrum) can be stored.

For breast pump systems 100 incorporating additional hygienic barriers, the backflow protector 108 can be positioned between the tubing 104 and the breast shield 105. In some embodiments, the backflow protector 108 can comprise a diaphragm housed within a sealed chamber, typically constructed from silicone or thermoplastic elastomers, preventing moisture or milk from entering the tubing 104 and contaminating the pump 103 and/or the motor 102. Users can customize their pumping experience using the adjustable suction control unit 109, which is either mechanically operated through a dial or electronically integrated within the electronic control panel 110. The electronic control panel 110 can be embedded in the main housing of the pump 103 and/or the motor 102, allows real-time adjustments of suction strength and cycle speed through tactile buttons or touchscreen interfaces.

Each component in the breast pump system 100 is designed for standardized compatibility, allowing interchangeability across different models and brands. The choice of materials ensures durability, hygiene, and comfort, with all milk-contact components meeting medical-grade safety standards. The structural form factor of the breast pump system 100 may vary depending on its intended use, with compact, lightweight designs favored for portable models, while larger, hospital-grade units feature reinforced enclosures and higher-powered motors for prolonged, intensive usage.

In some embodiments, an apparatus or device can be provided that comprises only a portion of the components described above regarding the breast pump system 100, such as only those components which are configured to come into contact with expressed fluids during use of the breast pump system 100. As used herein, the terms “milk-contacting components”, “milk-contacting elements”, and the like are used throughout the present disclosure interchangeably and refer to any component of the breast pump system 100 or other disclosed systems or apparatuses that are intended to come into contact with any expressed fluids (e.g., human breast milk, human colostrum, etc.) during normal use of the breast pump system 100 or other disclosed systems or apparatuses.

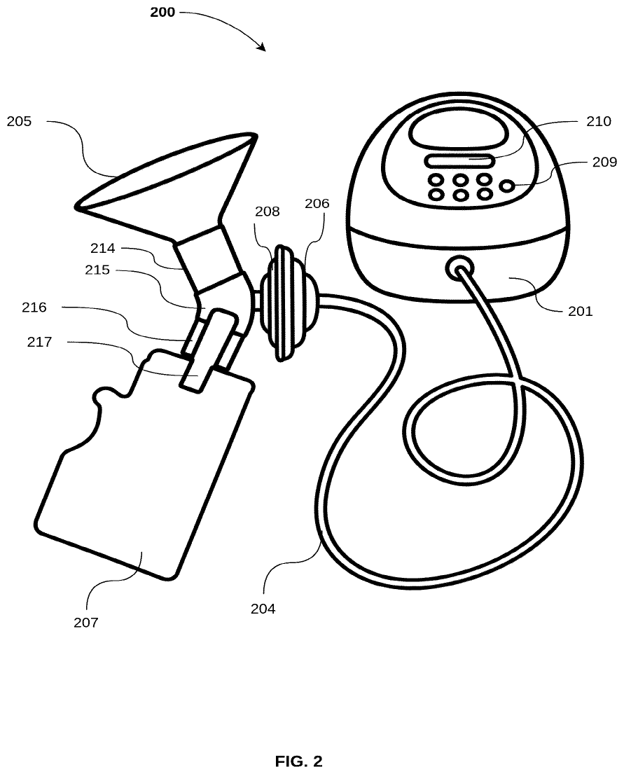

FIG. 2 illustrates an example of a breast pump system 200 which can be used to facilitate expression of human breastmilk or other lactation fluids. In some embodiments, the breast pump system 200 includes a set of components configured for facilitating expression of human breastmilk or other lactation fluids using, e.g., a double electric breast pump. In some embodiments, select components or elements of the breast pump system 200, such as ‘lactation fluid-contacting components’, can come in a kit in any suitable manner, including fully pre-assembled, fully disassembled, or only partially pre-assembled. In some embodiments, the kit can include components that are individually separated and sealed within a sterilized compact package designed to preserve hygiene while offering portability. The overall packaging of the kit can be discreet and small enough to be carried in a bag (e.g., purse, tote, diaper bag) or stored in a convenient location (e.g., the user's car, office, home, suitcase, or the like).

In some embodiments, the breast pump system 200 can comprise a breast pump housing 201 that contains therein a pump (not shown) and a motor (not shown). The breast pump housing 201 can include one or more controls 209 and one or more electronic displays 210. The controls 209 can be used by a lactating person to control one or more settings or characteristics of the breast pump system 200 before or during use thereof. In some embodiments, the lactating person can use the controls 209 to select a pre-programmed pumping intensity, a pre-programmed pumping style, a pre-programmed pumping duration, a pre-programmed pumping sequence, a pre-programmed pumping frequency, a pre-programmed pumping rate, other pumping settings, and/or the like. In some embodiments, the electronic display 210 can be used to display information about such pre-programmed characteristics or pumping settings to the lactating person.

The breast pump system 200 can further comprise a plurality of lactation fluid-contacting components or elements that each have distinct functions. The breast pump system 200 can include a breast shield 205, a breast shield connector 214, a body portion 215, a duckbill connector 216, and a duckbill valve 218. In some embodiments, the breast pump system 200 can further include a backflow protector 208. In some embodiments, the backflow protector 208 can comprise a pumping membrane 206 covering the backflow protector 208. In some embodiments, the breast pump system 200 can further comprise one or more adhesive strips 217.

In some embodiments, the body portion 215 can be fluidically coupled to the pump in the pump housing 201 via the backflow protector 208 and/or the pumping membrane 206 and further via a length of tubing 204 coupled to and disposed between the backflow protector 208/pumping membrane 206 and the pump housing 201.

In some embodiments, the breast shield 205 can be dimensioned and configured (e.g., sized, shaped, contoured) to ergonomically and/or sealingly fit onto, about, or against at least a portion of human breast tissue or at least a portion of a human breast. In some embodiments, the breast shield 205 can be dimensioned and configured according to a particular breast of a particular lactating person. In some embodiments, the breast shield 205 can be dimensioned and configured to, generally, fit a variety of shapes and sizes of breasts within a particular range of breast sizes and/or shapes. In some embodiments, the lactating person can determine, indicate, select, or otherwise associate themselves with one or more particular ranges of breast sizes and/or shapes, thereby ensuring that the lactating person receives a kit that includes one or more of the breast shield(s) 205 that are suitably dimensioned and configured with respect to the lactating person's specific breast(s) or breast tissue(s).

In some embodiments, the breast pump system 200 can further comprise the body portion 215 that is configured to be fluidically connected to a pump (not shown), which is not included in the kit. In some embodiments, the body portion 215 can comprise or define a central channel through which lactation fluid passes from the breast shield 205 to the lactation fluid collection container 207.

In some embodiments, the breast pump system 200 can further comprise the breast shield connector 214 that is configured to connect the breast shield 205 to the body portion 215.

In some embodiments, the breast pump system 200 can further comprise the duckbill valve 218 that is configured to fluidically connect the breast shield 405 to a lactation fluid collection container 207. In some embodiments, the lactation fluid collection container 207 can be provided in the kit, while in other embodiments the lactation fluid collection container 207 (e.g., single-use breastmilk collection bag or reusable breastmilk collection bottle) is provided by the lactating person and is not provided as part of the kit.

In some embodiments, the breast pump system 200 can further comprise the duckbill connector 216 that is configured to connect the duckbill valve 218 to the body portion 215.

In some embodiments, the breast pump system 200 can, optionally, further comprise the backflow protector 208 that is configured to connect the body portion 215 to the pump. The backflow protector 208 can be dimensioned and configured to prevent lactation fluid from flowing therethrough and to/into the pump, while allowing air to flow therethrough thereby causing a reduced air pressure (e.g., negative air pressure relative to ambient air pressure outside of the breast shield 205) to facilitate expression of lactation fluid from the breast tissue of the lactating person. In some embodiments, the pumping membrane 206 can cover or partially cover one side of the backflow protector 208. In some embodiments, the pumping membrane 206 can function as a barrier film between the backflow protector 208 and the pump/motor to ensure that lactation fluids do not touch or flow into the motor or pump.

In some embodiments, the breast pump system 200 can, optionally, further comprise one or more adhesive strips 217. In some embodiments, the one or more adhesive strips 217 can be or comprise one or more pieces of tape. In some embodiments, the one or more adhesive strips 217 can be compostable, biodegradable, recyclable, biobased, and/or the like.

In some embodiments, the breast shield connector 214 can be configured to connect the breast shield 205 to the body portion 215, thereby establishing a lactation fluid flow path by which lactation fluid is directed after being expressed from the breast tissue of the lactating person.

In some embodiments, the breast shield 205 is configured to slidably or rotatably receive at least a portion of the breast shield connector 214 therewithin in order to releasably or temporarily couple the breast shield 205 to the breast shield connector 214. In other embodiments, the breast shield connector 214 is configured to slidably or rotatably receive at least a portion of the breast shield 205 therewithin in order to releasably or temporarily couple the breast shield 205 to the breast shield connector 214.

In some embodiments, the body portion 215 is configured to slidably or rotatably receive at least a portion of the breast shield connector 214 therewithin in order to releasably or temporarily couple the body portion 215 to the breast shield connector 214. In other embodiments, the breast shield connector 214 is configured to slidably or rotatably receive at least a portion of the body portion 215 therewithin in order to releasably or temporarily couple the body portion 215 to the breast shield connector 214.

In some embodiments, the duckbill connector 216 is configured to slidably or rotatably receive at least a portion of the body portion 215 therewithin in order to releasably or temporarily couple the body portion 215 to the duckbill connector 216. In other embodiments, the body portion 215 is configured to slidably or rotatably receive at least a portion of the duckbill connector 216 therewithin in order to releasably or temporarily couple the body portion 215 to the duckbill connector 216.

In some embodiments, the duckbill connector 216 is configured to slidably or rotatably receive at least a portion of the duckbill valve 218 therewithin in order to releasably or temporarily couple the duckbill valve 218 to the duckbill connector 216. In other embodiments, the duckbill valve 218 is configured to slidably or rotatably receive at least a portion of the duckbill connector 216 therewithin in order to releasably or temporarily couple the duckbill valve 218 to the duckbill connector 216.

In some embodiments, the duckbill valve 218 can be or comprise a one-way valve that allows lactation fluid to flow towards therethrough and into the lactation fluid collection container, while preventing any flow of lactation fluid in the opposite direction.

In some embodiments, one or more of the breast shield 205, the breast shield connector 214, the body portion 215, the duckbill connector 216, and/or the duckbill valve 218 can be or comprise PHA-based materials, e.g., PHA or PHA-based polymer(s) or resin(s) derived from renewable sources. In some embodiments, the duckbill valve 205 can be or comprise an elastomeric polymer, such as elastomeric PHA or an elastomer comprising PHA, to allow for added flexibility and unidirectional sealing of the one-way valve. In some embodiments, the pumping membrane 206 can be or comprise any suitable compostable, food-safe material. In some embodiments, the one or more adhesive strips 217 can be or comprise a compostable material, a biodegradable material, or the like.

When assembled, the breast pump system 200 forms a closed pathway that connects securely to standard double electric pumps. The breast pump system 200 is engineered for use with suction pressures less than or equal to about 250 mmHg.

In some embodiments, the breast pump system 200 is configured to support a single breast pumping session of a single breast of a lactating person before the lactation fluid-contacting components must be discarded. In other embodiments, the breast pump system 200 is configured to support a single breast pumping session of both breasts of a lactating person before the lactation fluid-contacting components must be discarded. In other embodiments, the breast pump system 200 is configured to support multiple breast pumping sessions of both breasts of a lactating person before the lactation fluid-contacting components must be discarded. In some embodiments, the lactation fluid-contacting components are strictly single-use components and are not intended to be cleaned or sterilized for reuse.

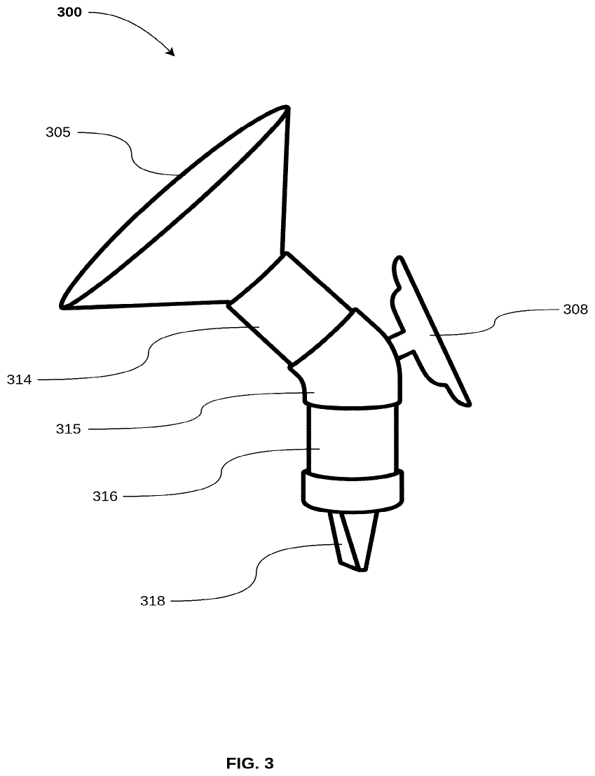

FIG. 3 illustrates a device 300 that comprises a set of components assembled for use with or in a breast pump system (e.g., 100, 200) to facilitate expression of human breastmilk or other lactation fluids. In some embodiments, the device 300 comprises a set of components configured for facilitating expression of human breastmilk or other lactation fluids using, e.g., a double electric breast pump. In some embodiments, the device 300 in any suitable manner, including fully pre-assembled, fully disassembled, or only partially pre-assembled. In some embodiments, the device 300 can be formed or assembled from components provided in a kit. In some embodiments, the kit can comprise components individually separated and sealed within a sterilized compact package designed to preserve hygiene while offering portability. The overall packaging of the kit can be discreet and small enough to be carried in a bag (e.g., purse, tote, diaper bag) or stored in a convenient location (e.g., the user's car, office, home, suitcase, or the like).

In some embodiments, the device 300 can comprise a plurality of components that each have distinct functions. The device 300 can comprise a breast shield 305, a breast shield connector 314, a body portion 315, a duckbill connector 316, and a duckbill valve 318. In some embodiments, the device 300 can further include a backflow protector 308. In some embodiments, the backflow protector 308 can comprise a thin film or barrier (not shown) covering at least a portion of the backflow protector 308. In some embodiments, the device 300 can further comprise one or more adhesive strips (not shown) configured to retain the duckbill valve 318 within or partially within a lactation fluid collection container (e.g., breastmilk collection bag).

In some embodiments, the breast shield 305 can be dimensioned and configured (e.g., sized, shaped, contoured) to ergonomically and/or sealingly fit onto, about, or against at least a portion of human breast tissue or at least a portion of a human breast. In some embodiments, the breast shield 305 can be dimensioned and configured according to a particular breast of a particular lactating person. In some embodiments, the breast shield 305 can be dimensioned and configured to, generally, fit a variety of shapes and sizes of breasts within a particular range of breast sizes and/or shapes. In some embodiments, the lactating person can determine, indicate, select, or otherwise associate themselves with one or more particular ranges of breast sizes and/or shapes, thereby ensuring that the lactating person receives a kit that includes one or more breast shield(s) 305 that are suitably dimensioned and configured with respect to the lactating person's specific breast(s) or breast tissue(s).

In some embodiments, the device 300 can further comprise the body portion 315 that is configured to be fluidically connected to a pump (not shown), which is not included in the device 300 and/or the kit of components provided therefore or therewith. In some embodiments, the body portion 315 can comprise or define a central channel through which lactation fluid passes from the breast shield 305 to the lactation fluid collection container.

In some embodiments, the device 300 can further comprise the breast shield connector 314 that is configured to connect the breast shield 305 to the body portion 315.

In some embodiments, the device 300 can further comprise the duckbill valve 318 that is configured to fluidically connect the breast shield 305 to a lactation fluid collection container (not shown). In some embodiments, the lactation fluid collection container can be provided with the device 300 and/or in the kit of components therewith or therefore, while in other embodiments the lactation fluid collection container (e.g., single-use breastmilk collection bag or reusable breastmilk collection bottle) is provided by the lactating person and is not provided with or as part of the device 300.

In some embodiments, the device 300 can further comprise the duckbill connector 316 that is configured to connect the duckbill valve 318 to the body portion 315.

In some embodiments, the device 300 can, optionally, further comprise the backflow protector 308 that is configured to connect the body portion 315 to the pump. The backflow protector 308 can be dimensioned and configured to prevent lactation fluid from flowing therethrough and to/into the pump, while allowing air to flow therethrough thereby causing a reduced air pressure (e.g., negative air pressure relative to ambient air pressure outside of the breast shield 305) to facilitate expression of lactation fluid from the breast tissue of the lactating person. In some embodiments, the backflow protector 308 can be provided with the thin film or barrier covering at least a portion of the backflow protector 308. In other embodiments, the device 300 can comprise the backflow protector 308 without the thin film or barrier. In some embodiments, the thin film or barrier film can be positioned between the backflow protector 308 and a pump/motor (neither shown nor included in the kit or as part of the device 300) to ensure that lactation fluids do not touch or flow into the motor or pump.

In some embodiments, the device 300 can, optionally, further comprise one or more adhesive strips (not shown). In some embodiments, the one or more adhesive strips can be or comprise one or more pieces of tape. In some embodiments, the one or more adhesive strips can be compostable, biodegradable, recyclable, biobased, and/or the like.

In some embodiments, the breast shield connector 314 can be configured to connect the breast shield 305 to the body portion 315 of the device 300, thereby establishing a lactation fluid flow path by which lactation fluid is directed after being expressed from the breast tissue of the lactating person.

In some embodiments, the breast shield 305 is configured to slidably or rotatably receive at least a portion of the breast shield connector 314 therewithin in order to releasably or temporarily couple the breast shield 305 to the breast shield connector 314. In other embodiments, the breast shield connector 314 is configured to slidably or rotatably receive at least a portion of the breast shield 305 therewithin in order to releasably or temporarily couple the breast shield 305 to the breast shield connector 314.

In some embodiments, the body portion 315 is configured to slidably or rotatably receive at least a portion of the breast shield connector 314 therewithin in order to releasably or temporarily couple the body portion 315 to the breast shield connector 314. In other embodiments, the breast shield connector 314 is configured to slidably or rotatably receive at least a portion of the body portion 315 therewithin in order to releasably or temporarily couple the body portion 315 to the breast shield connector 314.

In some embodiments, the duckbill connector 316 is configured to slidably or rotatably receive at least a portion of the body portion 315 therewithin in order to releasably or temporarily couple the body portion 315 to the duckbill connector 316. In other embodiments, the body portion 315 is configured to slidably or rotatably receive at least a portion of the duckbill connector 316 therewithin in order to releasably or temporarily couple the body portion 315 to the duckbill connector 316.

In some embodiments, the duckbill connector 316 is configured to slidably or rotatably receive at least a portion of the duckbill valve 318 therewithin in order to releasably or temporarily couple the duckbill valve 318 to the duckbill connector 316. In other embodiments, the duckbill valve 318 is configured to slidably or rotatably receive at least a portion of the duckbill connector 316 therewithin in order to releasably or temporarily couple the duckbill valve 318 to the duckbill connector 316.

In some embodiments, the duckbill valve 318 can be or comprise a one-way valve that allows lactation fluid to flow towards therethrough and into the lactation fluid collection container, while preventing any flow of lactation fluid in the opposite direction.

In some embodiments, one or more of the breast shield 305, the breast shield connector 314, the body portion 315, the duckbill connector 316, and/or the duckbill valve 318 can be or comprise PHA-based materials, e.g., PHA or PHA-based polymer(s) or resin(s) derived from renewable sources. In some embodiments, the duckbill valve 305 can be or comprise an elastomeric polymer, such as elastomeric PHA or an elastomer comprising PHA, to allow for added flexibility and unidirectional sealing of the one-way valve. In some embodiments, the barrier layer can be or comprise any suitable material, such as polymeric materials, PHA, compostable materials, food-safe materials, etc. In some embodiments, the one or more adhesive strips can be or comprise a compostable material, a biodegradable material, or the like.

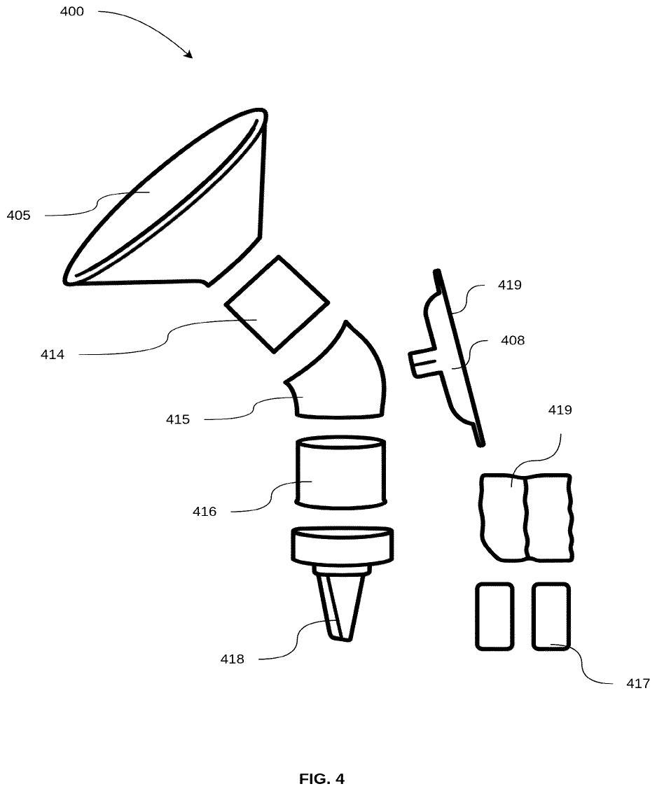

FIG. 4 illustrates an example of a kit 400 that includes a set of components configured for facilitating expression of human breastmilk or other lactation fluids using, e.g., a double electric breast pump. In some embodiments, components can come in the kit 400 in any suitable manner, including fully pre-assembled, fully disassembled, or only partially pre-assembled. In some embodiments, the components in the kit 400 can be individually separated and sealed within a sterilized compact package designed to preserve hygiene while offering portability. The overall packaging of the kit 400 can be discreet and small enough to be carried in a bag (e.g., purse, tote, diaper bag) or stored in a convenient location (e.g., the user's car, office, home, suitcase, or the like).

In some embodiments, the kit 400 can comprise a plurality of components that each have distinct functions. The kit 400 can include a breast shield 405, a breast shield connector 414, a body portion 415, a duckbill connector 416, and a duckbill valve 418. In some embodiments, the kit 400 can further include a backflow protector 408. In some embodiments, the backflow protector 408 can comprise a thin film 419 covering the backflow protector 408. In some embodiments, the kit 400 can further comprise one or more adhesive strips 417.

In some embodiments, the breast shield 405 can be dimensioned and configured (e.g., sized, shaped, contoured) to ergonomically and/or sealingly fit onto, about, or against at least a portion of human breast tissue or at least a portion of a human breast. In some embodiments, the breast shield 405 can be dimensioned and configured according to a particular breast of a particular lactating person. In some embodiments, the breast shield 405 can be dimensioned and configured to, generally, fit a variety of shapes and sizes of breasts within a particular range of breast sizes and/or shapes. In some embodiments, the lactating person can determine, indicate, select, or otherwise associate themselves with one or more particular ranges of breast sizes and/or shapes, thereby ensuring that the lactating person receives a kit 400 that includes one or more breast shield(s) 405 that are suitably dimensioned and configured with respect to the lactating person's specific breast(s) or breast tissue(s).

In some embodiments, the kit 400 can further comprise the body portion 415 that is configured to be fluidically connected to a pump (not shown), which is not included in the kit 400. In some embodiments, the body portion 415 can comprise or define a central channel through which lactation fluid passes from the breast shield 405 to the lactation fluid collection container.

In some embodiments, the kit 400 can further comprise the breast shield connector 414 that is configured to connect the breast shield 405 to the body portion 415.

In some embodiments, the kit 400 can further comprise the duckbill valve 418 that is configured to fluidically connect the breast shield 405 to a lactation fluid collection container (not shown). In some embodiments, the lactation fluid collection container can be provided in the kit 400, while in other embodiments the lactation fluid collection container (e.g., single-use breastmilk collection bag or reusable breastmilk collection bottle) is provided by the lactating person and is not provided as part of the kit 400.

In some embodiments, the kit 400 can further comprise the duckbill connector 416 that is configured to connect the duckbill valve 418 to the body portion 415.

In some embodiments, the kit 400 can, optionally, further comprise the backflow protector 408 that is configured to connect the body portion 415 to the pump. The backflow protector 408 can be dimensioned and configured to prevent lactation fluid from flowing therethrough and to/into the pump, while allowing air to flow therethrough thereby causing a reduced air pressure (e.g., negative air pressure relative to ambient air pressure outside of the breast shield 405) to facilitate expression of lactation fluid from the breast tissue of the lactating person. In some embodiments, the backflow protector 408 can be provided with the thin film 419 covering at least a portion of the backflow protector 408. In other embodiments, the backflow protector 408 provided in the kit 400 does not include the thin film 419, and instead the thin film 419 is provided by the lactating person. In some embodiments, the thin film 419 can function as a barrier film between the backflow protector 408 and a pump/motor membrane (membrane not included) to ensure that lactation fluids do not touch or flow into the motor or pump.

In some embodiments, the kit 400 can, optionally, further comprise one or more adhesive strips 417. In some embodiments, the one or more adhesive strips 417 can be or comprise one or more pieces of tape. In some embodiments, the one or more adhesive strips 417 can be compostable, biodegradable, recyclable, biobased, and/or the like.

In some embodiments, the breast shield connector 414 can be configured to connect the breast shield 405 to the body portion 415, thereby establishing a lactation fluid flow path by which lactation fluid is directed after being expressed from the breast tissue of the lactating person.

In some embodiments, the breast shield 405 is configured to slidably or rotatably receive at least a portion of the breast shield connector 414 therewithin in order to releasably or temporarily couple the breast shield 405 to the breast shield connector 414. In other embodiments, the breast shield connector 414 is configured to slidably or rotatably receive at least a portion of the breast shield 405 therewithin in order to releasably or temporarily couple the breast shield 405 to the breast shield connector 414.

In some embodiments, the body portion 415 is configured to slidably or rotatably receive at least a portion of the breast shield connector 414 therewithin in order to releasably or temporarily couple the body portion 415 to the breast shield connector 414. In other embodiments, the breast shield connector 414 is configured to slidably or rotatably receive at least a portion of the body portion 415 therewithin in order to releasably or temporarily couple the body portion 415 to the breast shield connector 414.

In some embodiments, the duckbill connector 416 is configured to slidably or rotatably receive at least a portion of the body portion 415 therewithin in order to releasably or temporarily couple the body portion 415 to the duckbill connector 416. In other embodiments, the body portion 415 is configured to slidably or rotatably receive at least a portion of the duckbill connector 416 therewithin in order to releasably or temporarily couple the body portion 415 to the duckbill connector 416.

In some embodiments, the duckbill connector 416 is configured to slidably or rotatably receive at least a portion of the duckbill valve 418 therewithin in order to releasably or temporarily couple the duckbill valve 418 to the duckbill connector 416. In other embodiments, the duckbill valve 418 is configured to slidably or rotatably receive at least a portion of the duckbill connector 416 therewithin in order to releasably or temporarily couple the duckbill valve 418 to the duckbill connector 416.

In some embodiments, the duckbill valve 418 can be or comprise a one-way valve that allows lactation fluid to flow towards therethrough and into the lactation fluid collection container, while preventing any flow of lactation fluid in the opposite direction.

In some embodiments, one or more of the breast shield 405, the breast shield connector 414, the body portion 415, the duckbill connector 416, and/or the duckbill valve 418 can be or comprise PHA-based materials, e.g., PHA or PHA-based polymer(s) or resin(s) derived from renewable sources. In some embodiments, the duckbill valve 405 can be or comprise an elastomeric polymer, such as elastomeric PHA or an elastomer comprising PHA, to allow for added flexibility and unidirectional sealing of the one-way valve. In some embodiments, the thin film 419 can be or comprise any suitable compostable, food-safe material. In some embodiments, the one or more adhesive strips 417 can be or comprise a compostable material, a biodegradable material, or the like.

When assembled, the kit 400, forms a closed pathway that connects securely to standard double electric pumps. The kit 400 contains components that are engineered for use with suction pressures less than or equal to about 250 mmHg.

In some embodiments, the kit 400 may comprise a sufficient number and type of components to support only a single breast pumping session of a single breast of a lactating person before the lactation fluid-contacting components must be discarded. In other embodiments, the kit 400 may comprise a sufficient number and type of components to support a single breast pumping session of both breasts of a lactating person before the lactation fluid-contacting components must be discarded. In other embodiments, the kit 400 may comprise a sufficient number and type of components to support multiple breast pumping sessions of both breasts of a lactating person before the lactation fluid-contacting components must be discarded. In some embodiments, the lactation fluid-contacting components provided in the kit 400 are strictly single-use components and are not intended to be cleaned or sterilized for reuse.

In some embodiments, one or more of the components, elements, or features of the systems (e.g., 100, 200), devices (e.g., 300) and kits (e.g., 400) described herein can be formed from biodegradable, compostable, and/or biobased materials, such as one or more from among: polylactic acid (PLA), polyhydroxyalkanoate (PHA), poly-3-hydroxybutyrate (PHB), polyhydroxyvalerate (PHV) and polyhydroxyhexanoate (PHH), starches, polycaprolactone, polybutylene-adipate-co-terephthalate, cellulose esters, cellulose acetate, nitrocellulose, celluloids, lignin-based polymer composites, polyglycolic acid (PGA), polybutylene succinate (PBS), polycaprolactone (PCL), poly(vinyl alcohol) (PVA), polybutylene adipate terephthalate (PBAT), BioPBS FD92 resin, BWC BF 90A resin, Ecopond Flex 162 resin, HCPT-1 triple laminate, HCFD-2 duplex laminate, Torise TRBF90 resin, variations thereof, or combinations thereof.

In some embodiments, one or more of the components, elements, or features of the systems (e.g., 100, 200), devices (e.g., 300) and kits (e.g., 400) described herein can be formed from other materials, such as one or more from among: a polymer, thermoplastic polymers, acrylonitrile-butadiene-styrene, polyurethane, acrylic, poly(acrylonitrile), polyolefins, polyvinyl chlorides, nylons, fluorocarbons, polystyrenes, polyethylene, ultra-high molecular weight polyethylene, polypropylene, polybutene, polymethylpentene, polyisoprene, polyethylene, ultra-high molecular weight polyethylene, polypropylene, ethylene-butene copolymers, ethylene-hexene copolymers, thermosetting plastics, polyimide (PI), poly amide (PA), poly amide imide (PAI), polypropylene (PP), polyethylene (PE), ethylene vinylacetate (EVA), poly(ethylene terephthalate) (PET), poly(vinyl acetate) (PVA), poly lactic-co-glycolic acid (PLGA), polylactic acid (PLA), polyamide (PA), acrylic adhesives, ultraviolet (UV)/electron beam (EB)/infrared (IR) curable resin, polyether ether ketone (PEEK), polyethylene naphthalate (PEN), polyethersulfone (PES), polyphenylene sulfide (PPS), polyphenylene oxide (PPO), copolymers thereof, variants thereof, or combinations thereof. In some embodiments, one or more of the components, elements, or features of the systems (e.g., 100, 200), devices (e.g., 300) and kits (e.g., 400) described herein can be formed from other materials, such as one or more from among: thermoplastic polymers, thermosetting polymers, acrylonitrile-butadiene-styrene, polyurethane, acrylic, poly(acrylonitrile), polyolefins, polyvinyl chlorides, nylons, fluorocarbons, polystyrenes, polyethylene, ultra-high molecular weight polyethylene, polypropylene, polybutene, polymethylpentene, polyisoprene, polyethylene, ultra-high molecular weight polyethylene, polypropylene, ethylene-butene copolymers, ethylene-hexene copolymers, thermosetting plastics, polyimide (PI), poly amide (PA), poly amide imide (PAI), polypropylene (PP), polyethylene (PE), ethylene vinylacetate (EVA), poly(ethylene terephthalate) (PET), poly-vinyl acetate (PVA), polyamide (PA), acrylic adhesives, ultraviolet (UV)/electron beam (EB)/infrared (IR) curable resin, polyether ether ketone (PEEK), polyethylene naphthalate (PEN), polyethersulfone (PES), polyphenylene sulfide (PPS), polyphenylene oxide (PPO), copolymers thereof, variants thereof, or any combination thereof.

In some embodiments, the components, elements, or features of the systems (e.g., 100, 200), devices (e.g., 300) and/or kits (e.g., 400) described herein can be used in additional or alternative systems, devices, and/or associated methods. For example, in some embodiments, the same or similar biodegradable or compostable lactation fluid-contacting components can be used in or with a manual pump device, a hand pump device, with multiple different breast shields/flanges, with freezer-grade lactation fluid collection containers, etc.

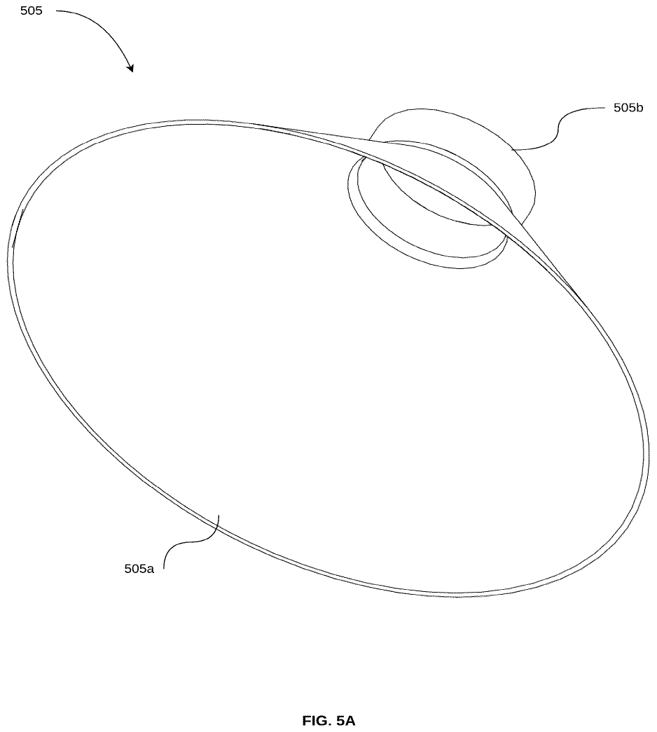

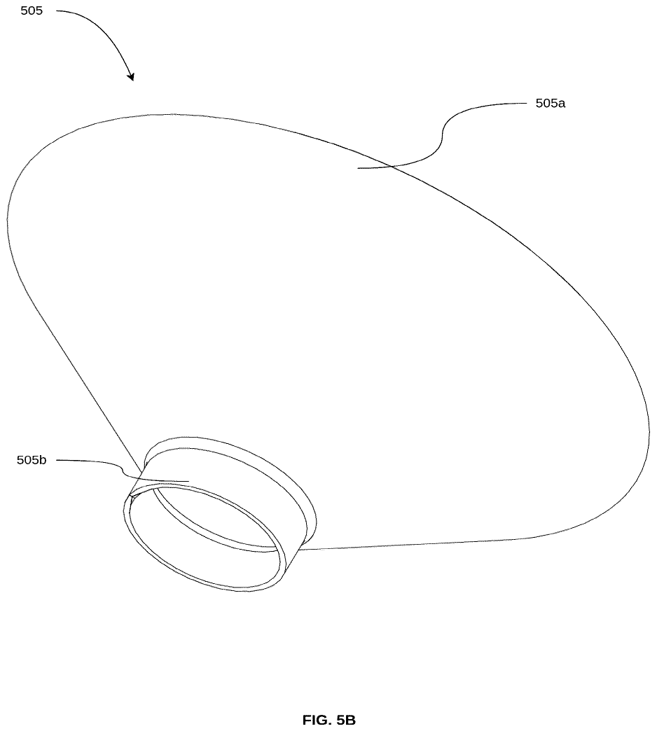

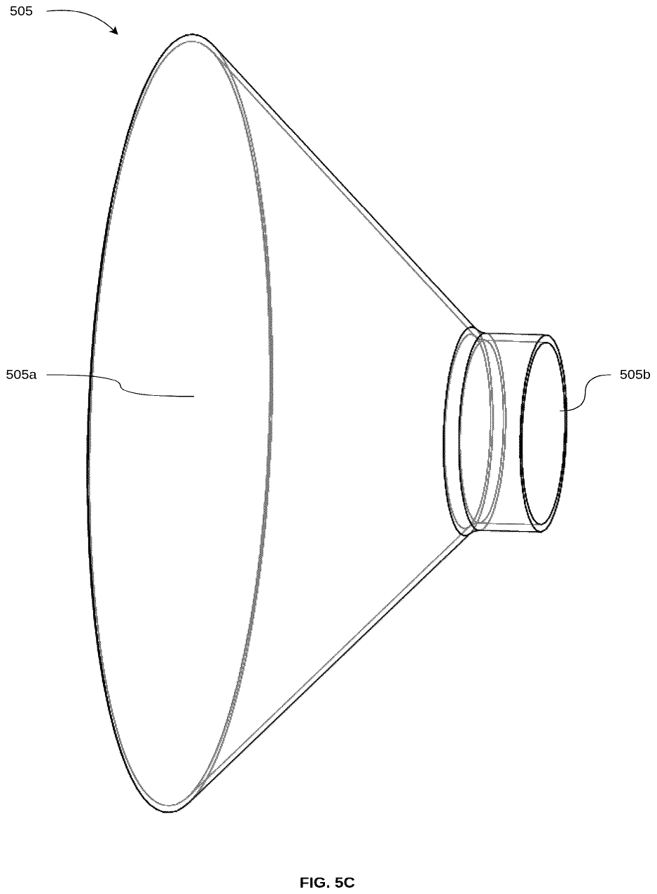

FIGS. 5A-5C illustrate, from a variety of perspectives, a breast shield 505, in accordance with an embodiment of the present disclosure. The illustrated breast shield 505 includes a breast tissue-contacting portion 505a and a pump-side portion 505b. In some embodiments, the breast tissue-contacting portion 505a has a generally concave form factor. In some embodiments, the breast tissue-contacting portion 505a has a generally cylindrical form factor. In some embodiments, the breast tissue-contacting portion 505a has a broad, funnel-like opening that is dimensioned and configured to receive a breast or a portion of a breast or breast tissue of a lactating person therewithin.

In some embodiments, the breast tissue-contacting portion 505a is dimensioned and configured that when a breast or a portion of a breast or breast tissue of a lactating person is disposed within a concavity or inner volume defined therein, a sufficiently air-tight seal is formed between an outer surface of the breast or breast tissue to facilitate enhanced expression of lactation fluids from the breast or breast tissue of the lactating person. In some embodiments, the breast tissue-contacting portion 505a is dimensioned and configured that when a breast or a portion of a breast or breast tissue of a lactating person is disposed within a concavity or inner volume defined therein, the breast tissue-contacting portion 505a of the breast shield 505 can have a form factor and dimensions suitable to allow for sufficient centering of the nipple and primary milk ducts of the breast or breast tissue within a center of the breast shield 505.

In some embodiments, the breast tissue-contacting portion 505a is dimensioned and configured such that when a breast or a portion of a breast or breast tissue of a lactating person is disposed within a concavity or inner volume defined therein, the breast tissue-contacting portion 505a of the breast shield 505 can have a form factor and dimensions suitable to allow for the nipple, areola, and/or primary milk ducts of the breast or breast tissue, which are generally centered within the breast shield 505, to move freely in response to pulsing suction intensity, which is often used as part of pre-programmed breast pumping sequences to mimic breastfeeding by a human infant.

In some embodiments, the pump-side portion 505b of the breast shield 505 is dimensioned and configured such that when a breast or a portion of a breast or breast tissue of a lactating person is disposed within the breast tissue-contacting portion 505a of the breast shield 505, the pump-side portion 505b of the breast shield 505 has a form factor and dimensions (e.g., sufficiently large inner diameter) to allow for the nipple, areola, and/or primary milk ducts of the breast or breast tissue, which are generally centered within the breast shield 505, to move freely within or through the pump-side portion 505b (i.e., opening at the narrow end of the conical/funnel shape of the breast shield 505) in response to pulsing suction intensity.

In some embodiments, the pump-side portion 505b is dimensioned and configured as a coupling sleeve that either receives a portion of a breast shield connector (e.g., 414) or which is disposed within a portion of the breast shield connector. In some embodiments, the pump-side portion 505b can have an outer diameter that is approximately the same as an inner diameter of a breast shield connector (e.g., 414), such that when the pump-side portion 505b is disposed or partially disposed within an inner portion of the breast shield connector (e.g., 414), the pump-side portion 505b of the breast shield 505 is frictionally retained within the breast shield connector (e.g., 414) due in part to the surface characteristics of the two components and also due to the similarity of the outer diameter of the pump-side portion 505b of the breast shield 505 relative to the inner diameter of the breast shield connector (e.g., 414).

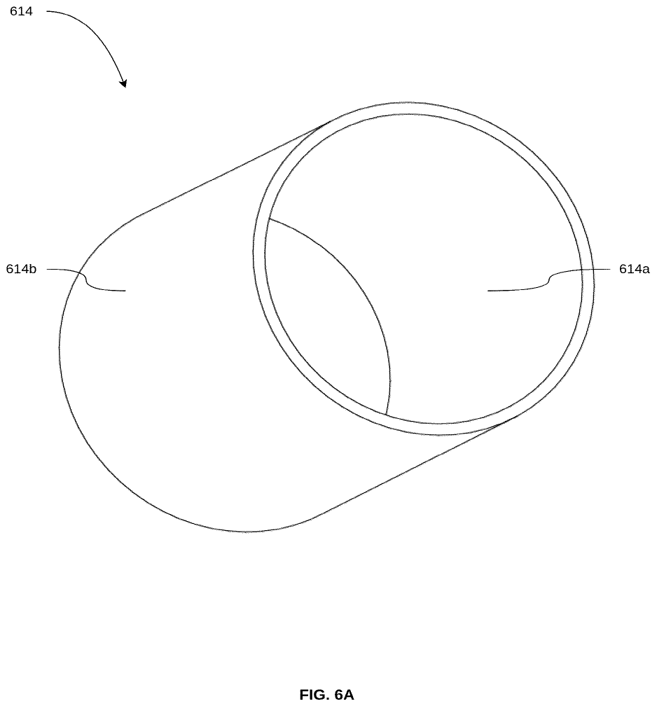

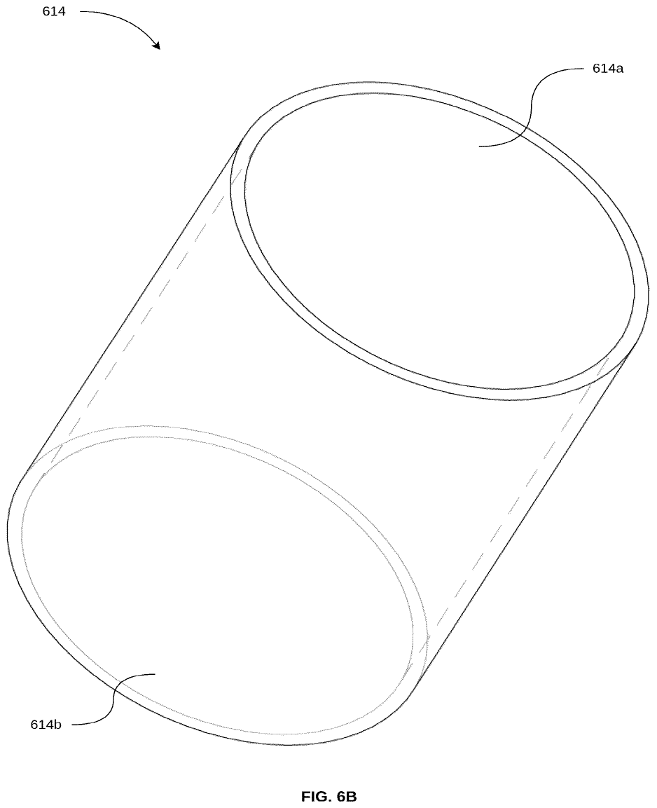

FIGS. 6A and 6B illustrate, from a variety of perspectives, a breast shield connector 614, in accordance with an embodiment of the present disclosure. The illustrated breast shield connector 614 includes a first end 614a and a second end 614b. In some embodiments, the breast shield connector 614 has a generally columnar form factor. In some embodiments, the first end 614a of the breast shield connector 614 has a particular form factor and one or more particular dimensions suitable to accommodate frictional coupling of the breast shield connector 614 to a breast shield (e.g., 505) or a component of a breast shield (e.g., pump-side portion 505b).

In some embodiments, the first end 614a is dimensioned and configured as a coupling sleeve that either receives a portion of the breast shield (e.g., 505) or which is disposed within a portion of a breast shield (e.g., 505). In some embodiments, the first end 614a can have an outer diameter that is approximately the same as an inner diameter of a breast shield (e.g., 505), such that when the first end 614a of the breast shield connector 614 is disposed or partially disposed within an inner portion of a pump-side element (e.g., 505b) of a breast shield (e.g., 505), the first end 614a of the breast shield connector 614 is frictionally retained within the pump-side portion (e.g., 505b) of the breast shield (e.g., 505) due in part to the surface characteristics of the two components and also due to the similarity of the inner diameter of the pump-side portion (e.g., 505b) of the breast shield (e.g., 505) relative to the outer diameter of the first end 614a of the breast shield connector 614.

In some embodiments, the second end 614b is dimensioned and configured as a coupling sleeve that either receives a portion of a body portion (e.g., 415) or which is disposed within a portion of a body portion (e.g., 415). In some embodiments, the second end 614b can have an outer diameter that is approximately the same as an inner diameter of a first end of a body portion (e.g., 415), such that when at least a portion of the first end of the body portion (e.g., 415) is disposed or partially disposed within an inner portion of the second end 614b of the breast shield connector 614, the first end of the body portion (e.g., 415) is frictionally retained within the second end 614b of the breast shield connector 614 due in part to the surface characteristics of the two components and also due to the similarity of the outer diameter of the second end of the breast shield connector 614b relative to the inner diameter of the first end of the body portion (e.g., 415).

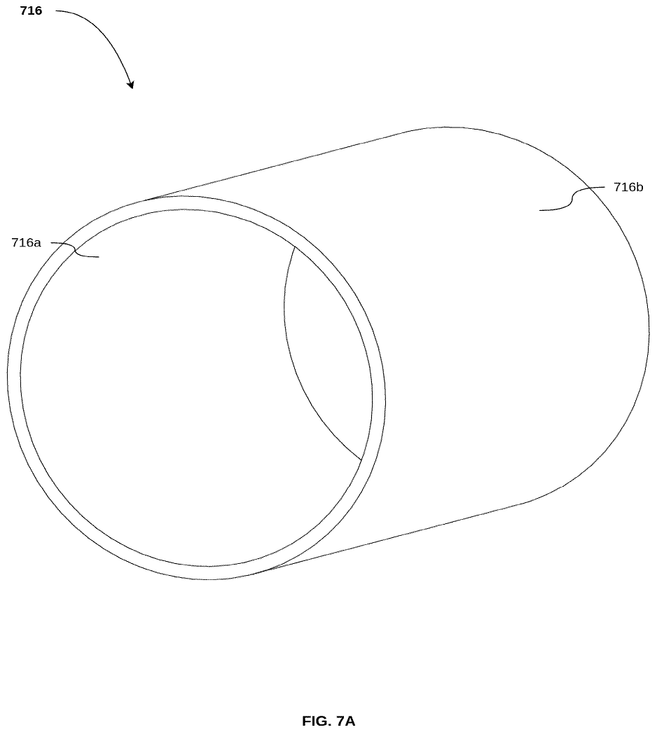

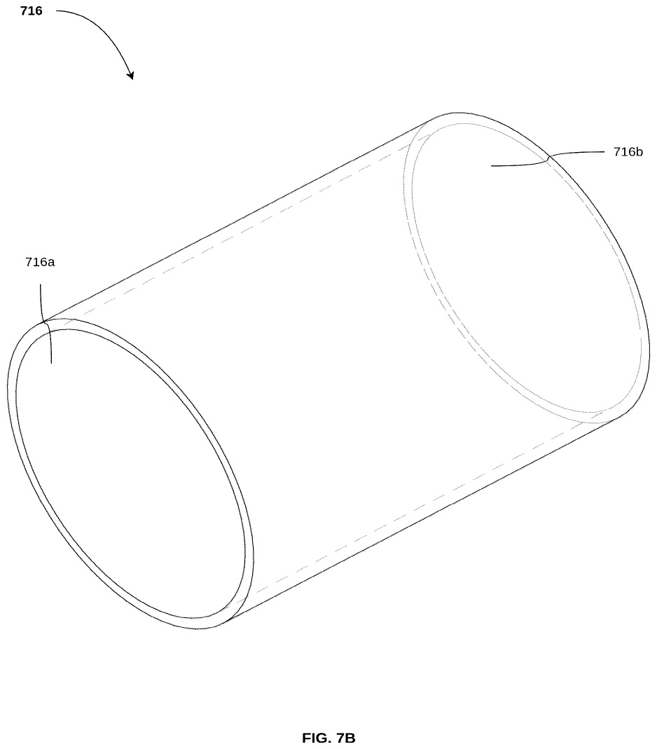

FIGS. 7A and 7B illustrate, from a variety of perspectives, a duckbill connector 716, in accordance with an embodiment of the present disclosure. The illustrated duckbill connector 716 includes a first end 716a and a second end 716b. In some embodiments, the duckbill connector 716 has a generally columnar form factor. In some embodiments, the first end 716a of the duckbill connector 716 has a particular form factor and one or more particular dimensions suitable to accommodate frictional coupling of the duckbill connector 716 to a body portion (e.g., 615) or a component of a body portion (e.g., second end 615b).

In some embodiments, the first end 716a is dimensioned and configured as a coupling sleeve that either receives a portion of the body portion (e.g., 415) or which is disposed within the body portion (e.g., 415). In some embodiments, the first end 716a can have an outer diameter that is approximately the same as an inner diameter of a body portion (e.g., 415), such that when the first end 716a of the duckbill connector 716 is disposed or partially disposed within an inner portion of a body portion (e.g., 415), the first end 716a of the duckbill connector 716 is frictionally retained within the body portion (e.g., 415) due in part to the surface characteristics of the two components and also due to the similarity of the inner diameter of the body portion (e.g., 415) relative to the outer diameter of the first end 716a of the duckbill connector 716.

In some embodiments, the second end 716b of the duckbill connector 716 is dimensioned and configured as a coupling sleeve that either receives a portion of a duckbill valve (e.g., 418) or which is disposed within a portion of a duckbill valve (e.g., 418). In some embodiments, the second end 716b of the duckbill connector 716 can have an outer diameter that is approximately the same as an inner diameter of a first end of a duckbill valve (e.g., 418), such that when at least a portion of the first end of the duckbill valve (e.g., 418) is disposed or partially disposed within an inner portion of the second end 716b of the duckbill connector 716, the first end of the duckbill valve (e.g., 418) is frictionally retained within the second end 716b of the duckbill connector 716 due in part to the surface characteristics of the two components and also due to the similarity of the outer diameter of the second end 716b of the duckbill connector 716 relative to the inner diameter of the first end of the duckbill valve (e.g., 418).

In other embodiments, the second end 716b of the duckbill connector 716 is dimensioned and configured to be disposed within a portion of a duckbill valve (e.g., 418). In some embodiments, the second end 716b of the duckbill connector 716 can have an inner diameter that is approximately the same as an outer diameter of a first end of a duckbill valve (e.g., 418), such that when at least a portion of the second end 716b of the duckbill valve 716 is disposed or partially disposed within a duckbill valve (e.g., 418), the second end 716b of the duckbill connector 716 is frictionally retained within the duckbill valve (e.g., 418) due in part to the surface characteristics of the two components and also due to the similarity of the inner diameter of the second end 716b of the duckbill connector 716 relative to the outer diameter of the first end of the duckbill valve (e.g., 418).

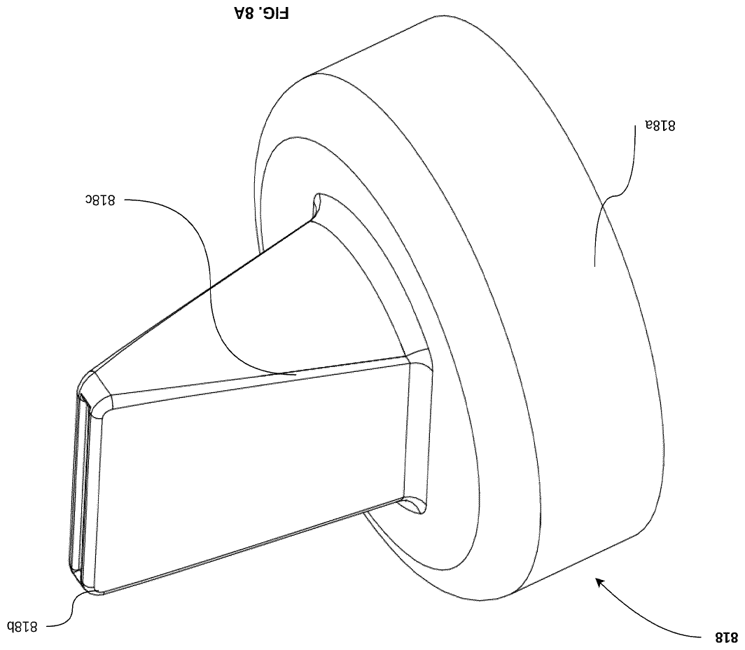

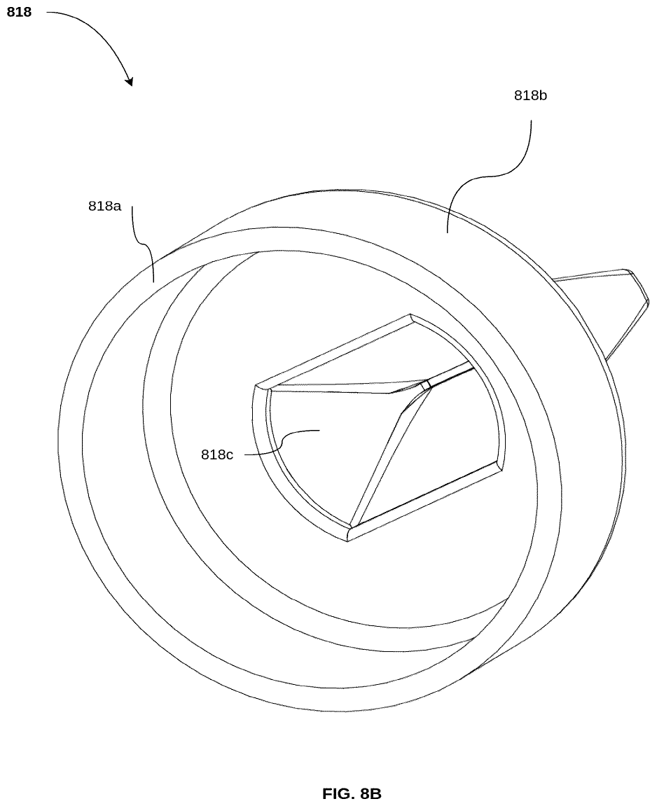

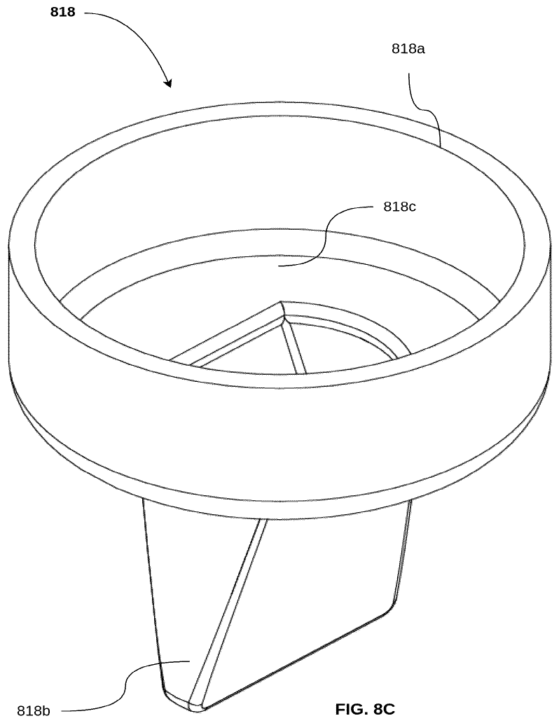

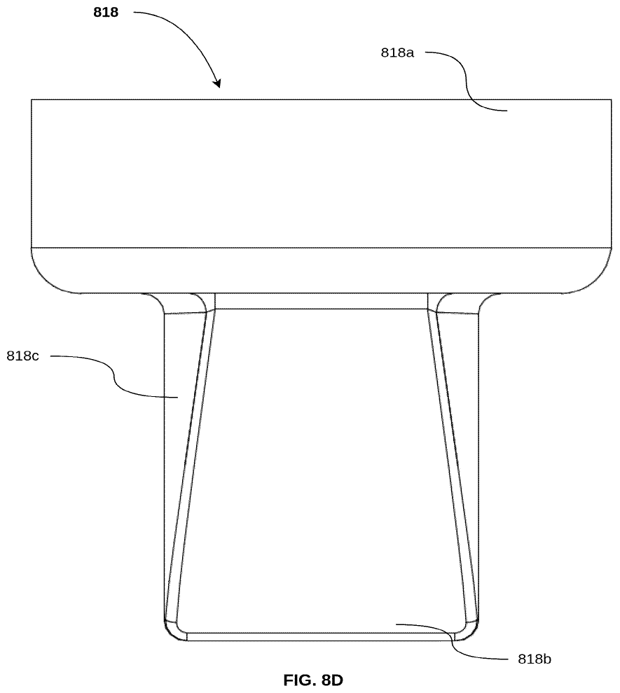

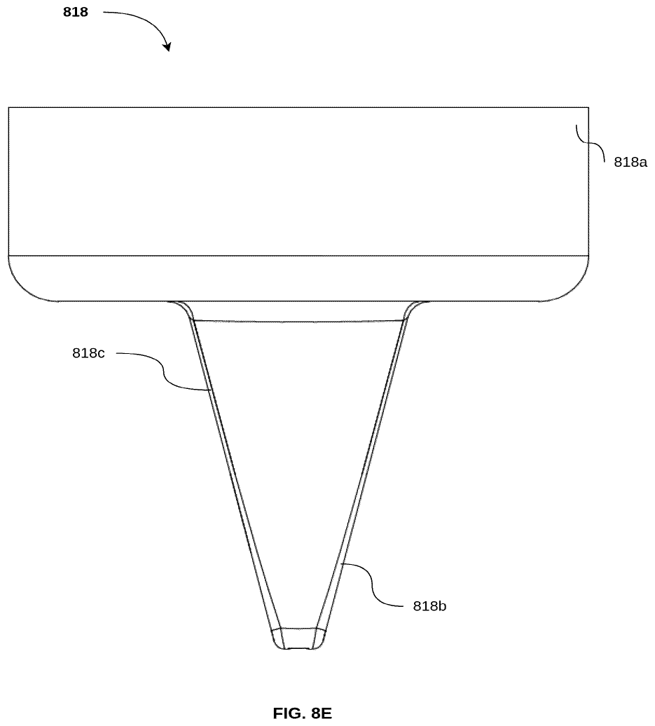

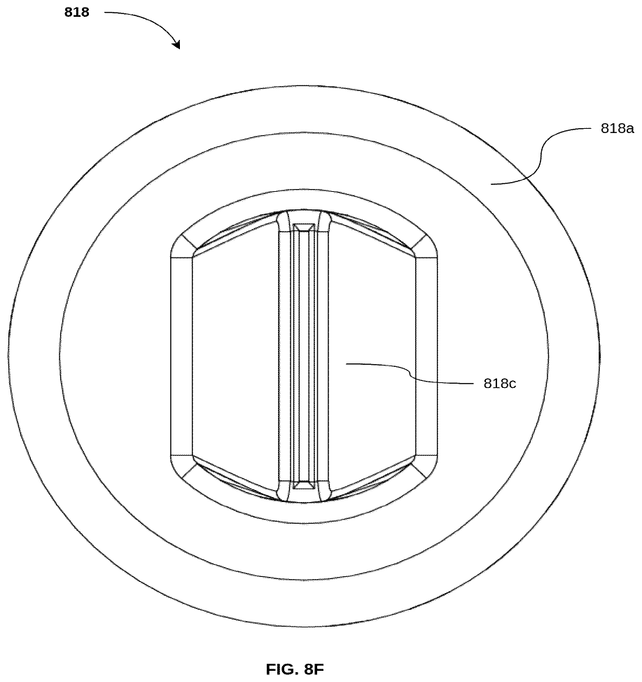

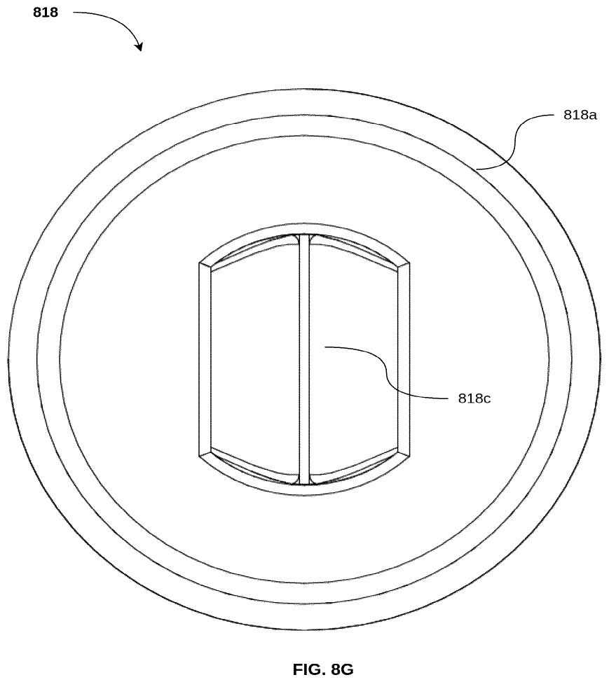

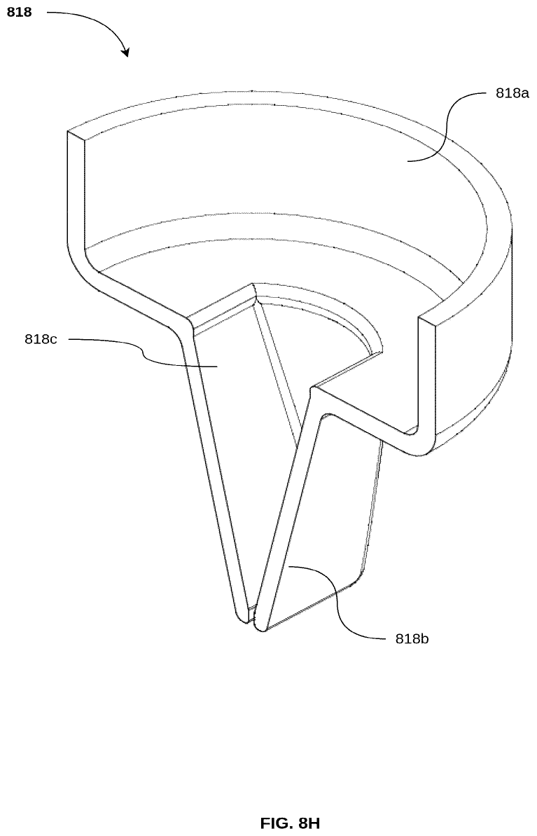

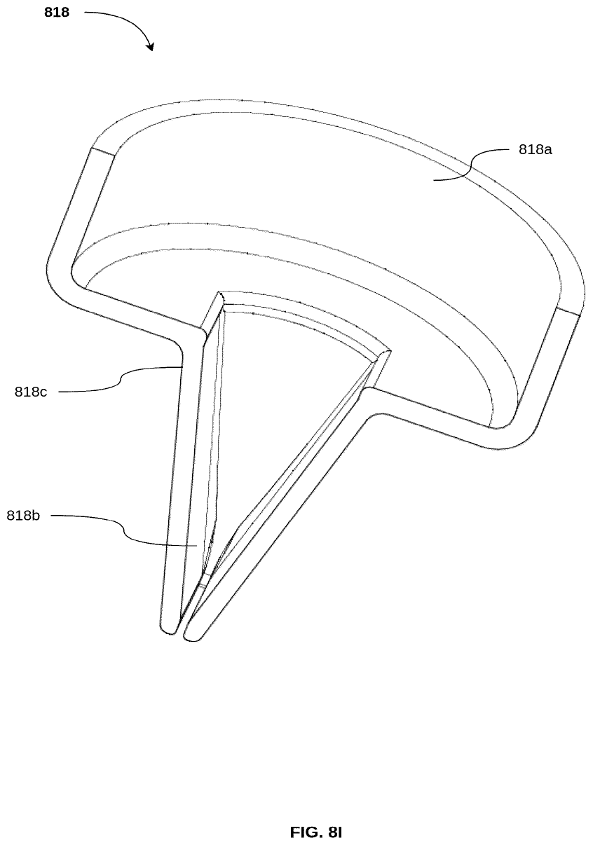

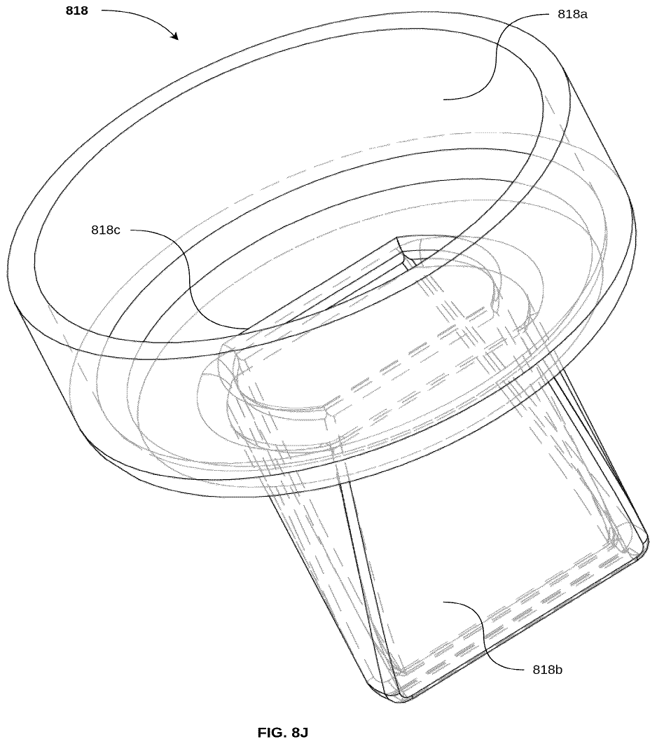

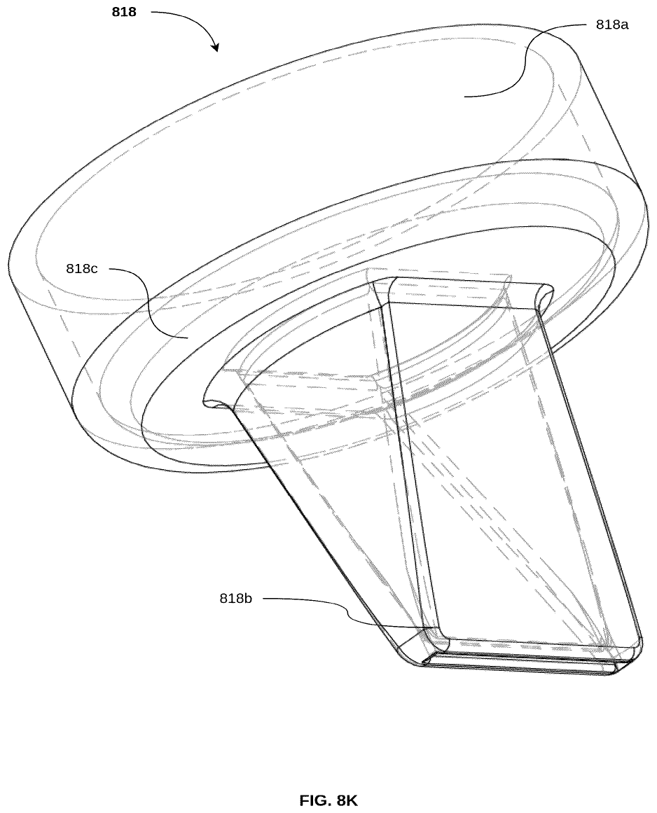

FIGS. 8A-8K illustrate, from a variety of perspectives, a duckbill valve 818, in accordance with an embodiment of the present disclosure. The illustrated duckbill valve 818 includes a first end 818a and a second end 818b as well as a one-way valve portion 818c. In some embodiments, the first end 818a of the duckbill valve 818 has a particular form factor that forms the one-way valve 818c. In some embodiments, the one-way valve 818c of the duckbill valve 818 is formed by a narrow channel or slit opening at the first end 818a of the duckbill valve 818. In some embodiments, the narrowness of the channel or slit opening allows for lactation fluid to flow unidirectionally through the one-way valve 818c due in part to the pumping/suction force exerted by the pump on the lactation fluid expressed from the breast of the lactating person, and in certain circumstances also due to gravity due to the angle and positioning of the duckbill valve 818 relative to the breast shield (e.g., 505) and other components along the lactation fluid flow path between the breast shield (e.g., 505) and the lactation fluid collection container (e.g., breastmilk collection bag).

In some embodiments, the second end 818b of the duckbill valve 818 is dimensioned and configured (e.g., by having a form factor and one or more particular dimensions) to accommodate frictional coupling of the duckbill valve 818 to a duckbill connector (e.g., 716) or a component thereof (e.g., second end 716b).

In some embodiments, the second end 818b is dimensioned and configured as a coupling sleeve that receives a portion of the duckbill connector (e.g., 716). In some embodiments, the second end 818b can have an inner diameter that is approximately the same as an outer diameter of the duckbill connector (e.g., 716), such that when a portion of the duckbill connector (e.g., 716) is disposed or partially disposed within the second end 818b of the duckbill valve 818, the duckbill connector (e.g., 716) is frictionally retained within the second end 818b of the duckbill valve 818 due in part to the surface characteristics of the two components and also due to the similarity of the inner diameter of the second end 818b of the duckbill valve 818 relative to the outer diameter of the duckbill connector (e.g., 716).

Conventional breast pump systems typically utilize unitary components that are difficult to clean, recycle, or dispose of in an environmentally responsible manner. There is a need for modular designs that allow for simplified manufacturing, improved sustainability, and user customization while maintaining structural integrity during operation.

For example, breast pump flanges and other breast pump system/device components are generally formed as one-piece components, e.g., made of medical-grade materials such as polypropylene or silicone. During use, lactation fluids sch as milk may contact interior surfaces of the flange and/or other components, which may require thorough cleaning between pumping sessions. Because flanges often include narrow geometries, curved interiors, and integrated ducts, they can be difficult to fully access and clean, creating potential for residue buildup and bacterial growth.

Typically, users rely on disassembly, washing, soaking, steaming, or boiling to ensure hygiene of milk-contacting components. This process is time-consuming and inconsistent, especially for caregivers who pump frequently. The lack of modularity also limits replacement options.

Additionally, there are challenges with balancing sustainability and convenience. Fully reusable systems require maintenance and carry cleaning burdens. There remains a need for a modular breast pump system that allows flexible assembly, selective reuse, disposable components, and hygienic isolation of milk-contacting surfaces without compromising performance.

Other embodiments described herein comprise breast pump systems and devices that may incorporate modular components to enhance flexibility, ease of assembly, and sustainability. In certain embodiments, one or more parts of the breast pump, such as the flange, are configured to be separable into multiple subcomponents that can be joined together prior to use. For example, the flange may be divided into two substantially symmetrical halves along a vertical plane. These halves can be individually manufactured, packaged, and transported, and subsequently assembled by the user to form a complete flange. The modular design facilitates reduced material usage, improved recyclability, and simplified cleaning or disposal. Additionally, the modular nature allows for interchangeability of parts, enabling customization of fit and function for different users.

Various mechanisms may be employed to secure the modular components together. In some embodiments, the two flange halves include complementary interlocking features. One flange half may include one or more longitudinal ridges extending along its mating edge, while the opposing flange half includes corresponding channels. During assembly, the ridges slide into the channels, creating a frictional or snap-fit engagement that maintains alignment and structural integrity during use. Alternatively, the flange halves may be held together by an elastic fastener, such as a band, ring, or strap, disposed circumferentially around the exterior of the assembled flange. The elastic member applies radial compression to the halves, preventing separation under normal operating conditions. The fastener may be formed from a biodegradable or compostable elastomeric material to maintain the overall sustainability of the system. In some embodiments, the joining mechanism may include clips, latches, or adhesive strips that provide temporary or semi-permanent attachment. These features may be integrated into the flange halves or provided as separate components. The joining mechanisms are designed to withstand the mechanical stresses encountered during pumping while allowing for easy disassembly for cleaning, recycling, or disposal.

In some embodiments, a breast pump system is provided that comprises modular components, such as a flange, that can be separated into multiple subcomponents and joined together prior to use. Various joining mechanisms are disclosed, including interlocking features, elastic fasteners, and other attachment means. The modular design facilitates reduced material usage, improved recyclability, and ease of cleaning or disposal.

In some embodiments, the breast pump system includes a modular component, such as a flange, that is configured to be separable into two substantially symmetrical halves, e.g., along a vertical plane. These halves may be individually manufactured, packaged, and transported, and subsequently assembled by the user to form a complete flange. The modular nature allows for interchangeability of parts, enabling customization of fit and function for different users.

In some embodiments, the two flange halves may be secured together using various mechanisms. In some embodiments, one flange half includes one or more longitudinal ridges extending along its mating edge, while the opposing flange half includes corresponding channels. During assembly, the ridges slide into the channels, creating a frictional or snap-fit engagement that maintains alignment and structural integrity during use.

In some embodiments, the flange halves are held together by an elastic fastener, such as a band, ring, or strap, disposed circumferentially around the exterior of the assembled flange. The elastic member applies radial compression to the halves, preventing separation under normal operating conditions. The fastener may be formed from a biodegradable or compostable elastomeric material to maintain sustainability.

In some embodiments, portions (e.g., halves) may include embedded magnets or magnetic inserts that attract and hold the halves together. In some embodiments, portions (e.g., halves) may comprise magnets that are disposed on or coupled to one or more of the portions (e.g., both halves) such that magnets on or within a first half are configured to align with magnets on or within a second half.

In some embodiments, portions (e.g., halves) may be held together using, e.g., a hook-and-loop (Velcro®-type) mechanism. For example, portions of the component can comprise one or more hook-and-loop interfaces, e.g., provided along the mating edges for quick attachment and detachment.

In some embodiments, portions (e.g., halves) may include protruding tabs and corresponding recesses that snap together when pressed.

In some embodiments, portions (e.g., halves) may include interlocking threads or bayonet-style fittings that engage upon rotation.

In some embodiments, portions (e.g., halves) may comprise one or more removable, pressure-sensitive adhesive strips that may be applied along the mating edges for temporary bonding.

In some embodiments, portions (e.g., halves) may be joined by applying localized heat to melt and fuse the edges, forming a semi-permanent bond.

In some embodiments, portions (e.g., halves) can comprise one or more fasteners, such as a clip made from compostable material, that may be used to clamp the halves together.

In some embodiments, portions (e.g., halves) may be held together using an elastic fastening device, such as an elastic band. The elastic fastening device may be overmolded onto one half or portion and stretched over the other half during assembly.

In some embodiments, mating edges of the different portions (e.g., halves) may include alternating finger-like projections that interlock when pressed together or which may be interleaved.

In some embodiments, portions (e.g., halves) may include sealing surfaces that create a vacuum lock when pressed together, aided by a small suction port.

These joining mechanisms are designed to withstand mechanical stresses encountered during pumping while allowing for easy disassembly for cleaning, recycling, or disposal. The components may be manufactured from recyclable polymers, biodegradable materials, or compostable composites to enhance environmental sustainability.

These joining mechanisms may also be added to other components of breast pump systems and devices.

The invention provides a breast pump system comprising modular components, such as a flange, duckbill valve, flange connector, duckbill connector, backflow preventer, and breastmilk collection container, that can be separated into multiple subcomponents and joined together prior to use. Various joining mechanisms are disclosed, including interlocking features, elastic fasteners, magnetic couplings, and other attachment means. The modular design facilitates reduced material usage, improved recyclability, and ease of cleaning or disposal.

In some embodiments, the flange is separable into two substantially symmetrical halves along a vertical plane. These halves may be joined using ridges and channels for sliding engagement, elastic fasteners disposed circumferentially, or snap-fit tabs. Alternative embodiments include magnetic coupling, hook-and-loop fasteners, rotational locking threads, adhesive strips, heat-sealable edges, biodegradable clips, overmolded elastic bands, interdigitated fingers, and vacuum-assisted seals.

In some embodiments, a duckbill valve may comprise a valve body and flexible sealing lips manufactured as separate pieces. The lips may attach via interference fit, snap-fit tabs, or elastic rings. Alternative embodiments include magnetic coupling, rotational locking, biodegradable clips, or adhesive bonding. Modular design allows replacement of worn sealing lips without discarding the entire valve.

In some embodiments, a flange connector may be split into two halves joined by ridges and channels, snap-fit tabs, or elastic bands. Alternative embodiments include hook-and-loop fasteners, interdigitated fingers, adhesive strips, vacuum-assisted seals, or magnetic inserts. Modular connectors enable interchangeability between different flange sizes and tubing diameters.

In some embodiments, a duckbill connector may include multiple segments joined by frictional engagement, elastic fasteners, magnetic inserts, rotational locking threads, or biodegradable clips. Modular connectors allow easy replacement of individual segments.

In some embodiments, a backflow preventer may comprise a housing and a movable diaphragm. The housing may be split into two halves joined by snap-fit tabs, ridges and channels, or elastic bands. Alternative embodiments include heat-sealable edges, hook-and-loop fasteners, interdigitated fingers, or magnetic coupling. Modular design facilitates disassembly for cleaning and diaphragm replacement.

In some embodiments, a breastmilk collection container may be provided that is modular. In some embodiments, the container may include a base and a removable upper collar. The collar may attach via threaded engagement, snap-fit tabs, or elastic sealing rings. Alternative embodiments include magnetic coupling, adhesive strips, or vacuum-assisted seals. Modular containers enable compatibility with different flange connectors and allow replacement of worn parts.

In some embodiments, modular components may be manufactured from recyclable polymers, biodegradable materials, or compostable composites to enhance environmental sustainability. Joining mechanisms are designed to withstand mechanical stresses encountered during pumping while allowing for easy disassembly for cleaning, recycling, or disposal.

In some embodiments, a breast pump system can be provided that comprises a flange configured to be separable into at least two subcomponents. In some embodiments, components (e.g., the flange) of the breast pump system can be configured to be separated/disassembled into multiple segments and joined/assembled together via a mechanism selected from among, e.g., ridges and channels, snap-fit tabs, elastic bands, hook-and-loop fasteners, interdigitated fingers, adhesive strips, threaded engagement, elastic sealing rings, vacuum-assisted seals, magnetic inserts, heat-sealable edges, or magnetic coupling.

In some embodiments, a modular breast pump system can be provided that comprises a duckbill valve having a valve body and flexible sealing lips. In some embodiments, the valve body and/or sealing lips can be configured to be separated/disassembled into multiple segments and joined/assembled together via a mechanism selected from among, e.g., ridges and channels, snap-fit tabs, elastic bands, hook-and-loop fasteners, interdigitated fingers, adhesive strips, threaded engagement, elastic sealing rings, vacuum-assisted seals, magnetic inserts, heat-sealable edges, or magnetic coupling.

In some embodiments, a breast pump system can be provided that comprises a flange connector configured to couple a flange to a collection container. In some embodiments, the flange connector can be configured to be separated/disassembled into multiple segments and joined/assembled together via a mechanism selected from among, e.g., ridges and channels, snap-fit tabs, elastic bands, hook-and-loop fasteners, interdigitated fingers, adhesive strips, threaded engagement, elastic sealing rings, vacuum-assisted seals, magnetic inserts, heat-sealable edges, or magnetic coupling.

In some embodiments, a breast pump system can be provided that comprises a backflow preventer having a housing and a diaphragm. In some embodiments, the housing for the diaphragm of the backflow preventor can be configured to be separated/disassembled into multiple segments and joined/assembled together via a mechanism selected from among, e.g., ridges and channels, snap-fit tabs, elastic bands, hook-and-loop fasteners, interdigitated fingers, adhesive strips, threaded engagement, elastic sealing rings, vacuum-assisted seals, magnetic inserts, heat-sealable edges, or magnetic coupling.

In some embodiments, a breast pump system can be provided that comprises a breastmilk collection container having a base and a removable upper collar. In some embodiments, the breastmilk collection container, base and/or removable upper collar can be configured to be separated/disassembled into multiple segments and joined/assembled together via a mechanism selected from among, e.g., ridges and channels, snap-fit tabs, elastic bands, hook-and-loop fasteners, interdigitated fingers, adhesive strips, threaded engagement, elastic sealing rings, vacuum-assisted seals, magnetic inserts, heat-sealable edges, or magnetic coupling.

In some embodiments, portions (e.g., halves) of a modular component can be manufactured from biodegradable or compostable material. In some embodiments, the sealing lips are replaceable independently of the valve body. In some embodiments, the flange connector segments are interchangeable to accommodate different tubing diameters. In some embodiments, the diaphragm is replaceable without discarding the housing. In some embodiments, the removable upper collar is compatible with multiple flange sizes. In some embodiments, the joining mechanism comprises a vacuum-assisted seal for leak prevention. In some embodiments, the elastic fastener is formed from a compostable elastomeric material. In some embodiments, portions (e.g., halves) of a modular component can be configured for disassembly to facilitate recycling. In some embodiments, portions (e.g., halves) of a modular component can be packaged separately for compact storage. In some embodiments, a modular design may reduce material usage compared to unitary components.

In some embodiments, a breast pump system may include one or more disposable liners configured to cover inner surfaces of components that contact breast milk. These liners may provide a hygienic barrier between the milk-contacting surfaces and the milk, reducing contamination risk and simplifying cleaning. The liners may be formed from thin, flexible, food-grade materials that are recyclable, biodegradable, or compostable. Liners may be pre-sterilized and packaged individually or in sets.

In some embodiments, the liners are designed for easy application and removal without compromising the functionality of the underlying component. They may include features such as elastic edges, adhesive strips, interlocking tabs, or drawstring-like closures to maintain position during use. After pumping, the liner can be removed and discarded, leaving the component clean and ready for reuse or recycling.

In some embodiments, a disposable liner may be configured to conform to the inner surface of the flange, including the tapered region that contacts the breast. In some embodiments, the liner comprises a thin, flexible sleeve with an elastic rim that stretches over the flange opening and secures around the outer edge. In some embodiments, adhesive-backed liners can be provided that are configured to adhere to the inner surface or liners with interlocking tabs that engage with grooves in the flange. In some embodiments, liner removal is accomplished by peeling or sliding the liner off the flange or other component.

In some embodiments, a liner for the duckbill valve may be shaped as a small pouch or sheath that covers the inner surfaces of the valve body and the flexible lips. In one embodiment, the liner includes a narrow neck that fits snugly around the valve inlet and an expandable body that lines the milk-contacting surfaces. The liner may be applied by inserting it into the valve body and stretching the neck over the inlet. Removal involves pulling the liner out by its neck, minimizing contact with residual milk.

In some embodiments, the flange connector liner may be a tubular sleeve that lines the interior passage through which milk flows. In one embodiment, the liner includes elastic ends that grip the connector openings. In some embodiments, liners can be provided that comprise adhesive edges or snap-fit rings that secure the liner in place. In some embodiments, liner removal is achieved by pulling the liner out through either end of the connector.

In some embodiments, similar to the flange connector, the duckbill connector liner may be a flexible tube that lines the internal milk-contacting surfaces. In some embodiments, a liner for a flange connector may include elastic or adhesive features for secure placement. In some embodiments, the liner is pre-formed to match the connector's internal geometry for precise fit.

In some embodiments, a liner for the backflow preventer may be configured as a thin membrane or pouch that covers the diaphragm and inner housing surfaces. In some embodiments, the liner includes tabs or adhesive strips for secure placement. In some embodiments, liner removal involves lifting the liner out by its tabs, leaving the diaphragm and housing clean.

In some embodiments, the collection container liner may be a flexible bag or pouch that fits inside the container and conforms to its shape. In some embodiments, the liner includes a collar that stretches over the container rim and secures under the flange connector. In some embodiments, liners can be provided which include drawstring closures or adhesive rims. After pumping, the liner containing the milk can be sealed and removed for storage or disposal, eliminating the need to wash the container.

In some embodiments, liners may be applied manually by stretching, sliding, or pressing into place. Some embodiments include alignment indicators or tabs to ensure proper positioning. In some embodiments, liner removal is designed to be quick and hygienic, often involving pulling tabs, peeling adhesive, or sliding the liner out without spilling milk. In some embodiments, liners may be compatible with modular components, allowing them to be installed before or after assembly.

In certain embodiments, the breast pump system is configured as a hybrid assembly in which selected components are reusable while others are designed for single use, which may include disposable, recyclable, and/or compostable materials. This approach optimizes sustainability and user convenience by reducing waste while maintaining hygiene. For example, the flange may be manufactured as a reusable component from durable, sterilizable material such as medical-grade silicone or rigid polymer. Prior to each use, a thin, flexible disposable liner is applied to the inner surface of the flange to create a hygienic barrier between the breast and the flange. After pumping, the liner is removed and discarded, leaving the flange clean and ready for reuse without requiring washing.

Similarly, the breastmilk collection container may be reusable, while a disposable liner in the form of a pouch or bag is positioned inside the container to receive expressed milk. The liner may include a collar that secures around the container rim or under the flange connector, ensuring leak-free operation. Once pumping is complete, the liner can be sealed and removed for storage or disposal, eliminating the need to clean the container. In contrast, components such as the duckbill valve or backflow preventer may be configured as single-use elements formed from recyclable or compostable plastics. These components are typically more difficult to clean thoroughly due to their internal geometries and small openings, making single-use construction advantageous for hygiene and convenience.

Hybrid embodiments may also include reusable connectors, such as a flange connector or duckbill connector, which are covered by disposable tubular liners that line the internal milk-contacting surfaces. This design allows the connector to remain clean after liner removal, reducing cleaning requirements. The liners may be applied manually by stretching over openings or sliding into internal passages, and may include elastic rims, adhesive strips, or interlocking tabs for secure placement. Removal is designed to be quick and hygienic, often involving pulling tabs or peeling adhesive edges. By combining reusable components with disposable liners and single-use parts, the system achieves a balance between sustainability, user convenience, and hygienic performance, while reducing the overall environmental impact compared to fully disposable systems.

In some embodiments, a modular breast pump system may include on or more components that are modular or at least one flange assembly defining a breast-contact surface and one or more optional liners or inserts. The flange assembly may consist of two or more interfacing components-for example, first and second portions-that when coupled form a substantially continuous internal contour.

The system is not limited to any particular number, orientation, or geometry of portions. The components may be symmetrical or asymmetrical, continuous or segmented, planar or curved, and may be configured to align vertically, horizontally, or along any arbitrary axis.

Each portion may contain features for structural integrity, fluid routing, or ergonomic comfort, and may be assembled, sealed, or retained in position by any form of mechanical, magnetic, adhesive, pneumatic, or other coupling method.

Coupling mechanisms between portions may include any type of interface capable of maintaining structural or fluid continuity. Examples include, but are not limited to:
magnetic or electromagnetic attraction,
snap-fit, latch, clip, or interference-fit connectors,
threaded, bayonet, or twist-lock joints,
elastic bands, gaskets, or seals,
hinges, pivots, or flexible connectors,
adhesive, cohesive, or solvent-bonded joints, and/or
mechanical fasteners such as screws, pins, or detents.

Any combination or sequence of such mechanisms may be used, and the invention encompasses all equivalents or future coupling technologies that perform the same or similar function.

The system may incorporate one or more liners, membranes, sleeves, or inserts that can serve as the milk-contacting surface of the interior of the flange. These elements may:
be rigid, flexible, or semi-flexible,
be disposable, reusable, or partially reusable,
be positioned inside, outside, or between structural components, and/or
attach by friction, suction, adhesive, mechanical retention, or any equivalent mechanism.

The liner material may include any natural or synthetic substance suitable for hygienic isolation, including but not limited to: thermoplastics, elastomers, fibers, textiles, papers, metals, composites, coated substrates, biodegradable polymers, and combinations thereof. Coatings may include hydrophobic, hydrophilic, antimicrobial, or barrier layers, whether organic or inorganic.

The modular breast pump system may be fabricated from any combination of medically compatible materials suitable for structural support, hygiene, comfort, and repeated sterilization.

Materials may be selected based on strength, elasticity, chemical resistance, sustainability, or tactile performance.

The flange assembly or structural components may be composed of any rigid, semi-rigid, or elastomeric material capable of maintaining form under negative pressure and contact with biological fluids.

Acceptable materials include, without limitation, medical-grade thermoplastics, such as polypropylene (PP), polyethylene (PE, including HDPE and LDPE), polycarbonate (PC), polyethylene terephthalate (PET), polyether ether ketone (PEEK), and polylactic acid (PLA); Thermoplastic elastomers (TPE), thermoplastic vulcanizates (TPV), silicone rubbers, and medical-grade urethanes; Fiber-reinforced or composite polymers combining rigidity and flexibility; Biopolymers or biodegradable resins, including PHB, PHA, and other plant-based or microbial-derived polymers; and Metals, ceramics, or glass-reinforced materials where rigidity or transparency is desired.

Flange components may be transparent, translucent, or opaque; may include colorant or texture for visual inspection or grip; and may incorporate coatings or surface treatments to reduce friction, enhance tactile comfort, or improve sterilization response. Surface finishes may include mechanical polishing, mold texturing, plasma treatment, or vapor deposition coatings to modify hydrophobicity or antimicrobial performance.

The liner, insert, sleeve, or barrier components may be fabricated from any flexible, conformable, or semi-rigid material capable of forming a hygienic, low-adhesion interface with expressed milk while maintaining dimensional stability during suction.

Suitable materials include, without limitation, molded natural or synthetic fibers, including cellulose pulp, bamboo fiber, or biopolymer-reinforced fiber blends; polymeric films or membranes, such as polyethylene (PE), polypropylene (PP), polyvinylidene chloride (PVDC), thermoplastic polyurethane (TPU), or multilayer laminates; elastomeric sheets or films, including silicone, latex-free rubbers, or medical-grade TPEs; paperboard or molded fiber substrates coated or laminated with hydrophobic layers, waxes, resins, or biodegradable barrier films; and/or composites or hybrid materials incorporating multiple layers to balance flexibility, barrier performance, and sustainability.

Surface treatments may include hydrophobic, oleophobic, antimicrobial, or biodegradable coatings, such as natural waxes (e.g., carnauba, candelilla, soy, or beeswax), plant-derived resins, or synthetic polymer coatings.

The liner material may be designed for single-use or limited reuse and may be sterilized by any physical, chemical, or irradiation method compatible with its structure.

The materials described herein are illustrative and non-limiting, and the invention encompasses all equivalents, derivatives, blends, and future-developed materials that perform the same or similar function in forming or isolating breast pump components.

Assembly of the modular breast pump system may occur by any manual, mechanical, or automated process in which components are aligned, mated, and secured to create an operational device. Assembly may involve sequential or simultaneous joining, partial or complete sealing, or pre-packaged sterile configuration.

During use, suction generated by a pump draws milk through the assembled components and into a collection container. Liners can be inserted or removed before or after each session. Components may be sterilized using any known or future sterilization or sanitization method.

According to other embodiments, variations, substitutions, and equivalents of the above embodiments of the breast pump component, devices, and systems can be provided that perform substantially the same function or achieve substantially the same result.

In some embodiments, multi-piece assemblies having more than two flange portions; flanges integrated with sensors, valves, or fluid-control elements; systems in which the liner or insert is self-supporting or collapsible; components formed as unitary flexible membranes; hybrid systems where only part of the assembly is reusable; alternate geometries (circular, oval, polygonal, contoured, or anatomical); and any configuration allowing interchange or upgrade of materials or parts.

The modular system provides for many different advantages, which may include (but are not limited to): broad compatibility with diverse materials and manufacturing techniques; ease of cleaning and improved hygiene through component separation; reduced environmental impact through biodegradable disposable components (liners/inserts); ergonomic and comfort adaptability; and future-proof design enabling integration with evolving pump technologies.

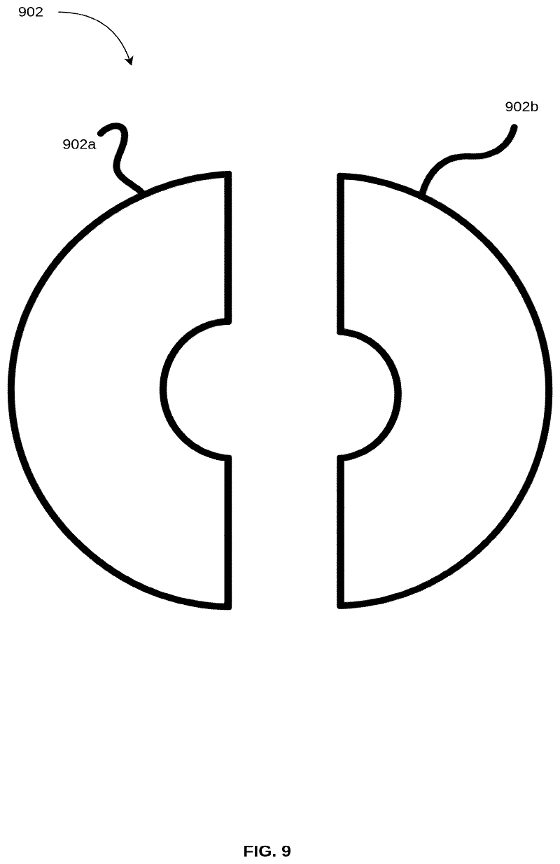

Referring now to FIG. 9-37, various components are illustrated for breast pump devices and systems and methods for using the same. For example, in FIG. 9, a breast pump flange 902 is illustrated from a top view that is modular in nature. The breast pump flange 902 comprises a first flange half 902a and a second flange half 902b. The two flange halves 902a, 902b, that are joinable at one or more connecting surfaces or mating surfaces. The two flange halves 902a, 902b can be provided to a user as part of a kit and assembled by the user before use. As described above, the two flange halves 902a, 902b can be coupled together using any suitable external coupling or fastening means, any suitable internal or integrated coupling or fastening means, or any suitable combination thereof.

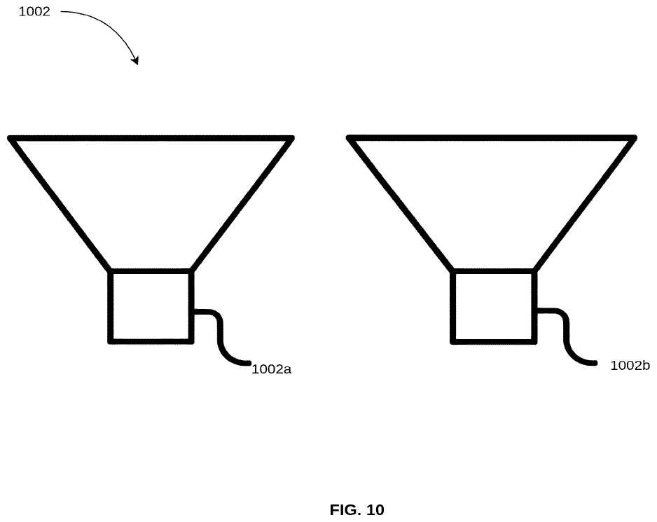

As illustrated in FIG. 10, a breast pump flange 1002 can be provided that comprises a first breast pump flange half 1002a and a second breast pump flange half 1002b that are configured to be coupled together to make an inner volume of the breast pump flange 1002 airtight. In some embodiments, the two halves (1002a, 1002b) of the breast pump flange 1002 can each define one or more concavities and one or more convexities. In some embodiments, the two halves (e.g., 1002a, 1002b) of the breast pump flange 1002 can each define one or more mating surfaces 1004a, 1004b.

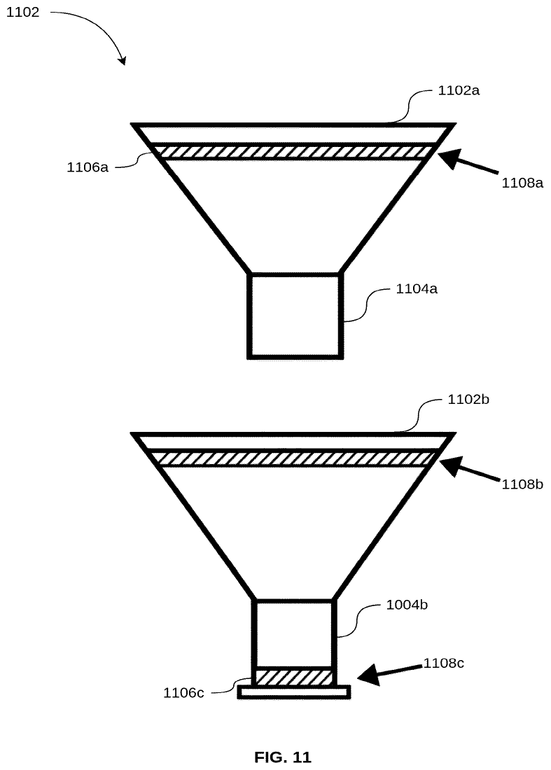

As illustrated in FIG. 11, a breast pump flange 1102 can be provided that comprises a first breast pump flange half 1102a and a second breast pump flange half 1102b that are configured to be coupled together to make an inner volume of the breast pump flange 1102 airtight. In some embodiments, the two halves (1102a, 1102b) of the breast pump flange 1102 can each define one or more concavities and one or more convexities. In some embodiments, the two halves (e.g., 1102a, 1102b) of the breast pump flange 1102 can each define one or more mating surfaces 1104a, 1104b. The breast pump flange halves 1102a, 1102b can further comprise or define one or more coupling mechanisms 1106a, 1106b, 1106c that are configured to retain one or more securing elements 1108a, 1108b, 1108c. As shown in FIG. 11, breast pump flange halves (e.g., 1102a) can comprise a single coupling mechanism 1106a, which may be or comprise a channel, groove, slot, or the like. As shown in FIG. 11, breast pump flange halves (e.g., 1102b) can comprise multiple coupling mechanisms 1106b, 1106c, which may be or comprise a channel, groove, slot, or the like. In some embodiments, the one or more securing elements 1108a, 1108b, 1108c can be or comprise an elastic band, a snap, a latch, a screw, a cylindrical connector, a mortise, a securing wire, a connecting loop, a removable collar, or the like.

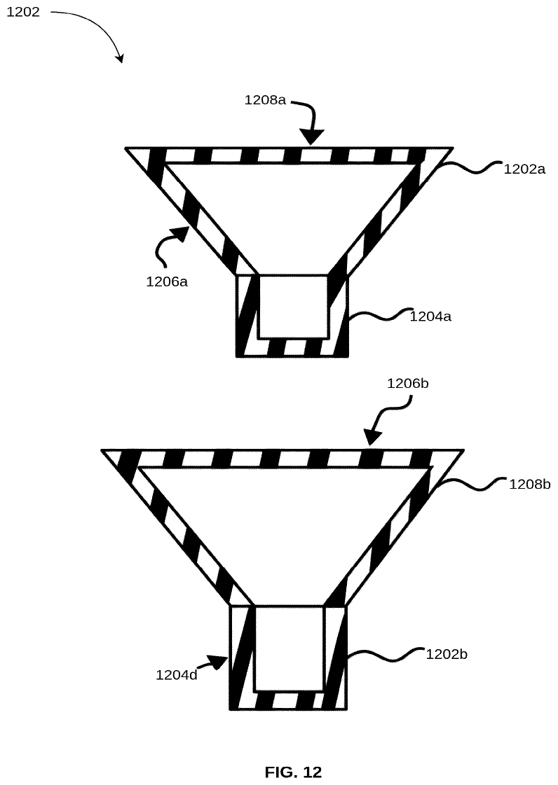

As illustrated in FIG. 12, a breast pump flange 1202 can be provided that comprises a first breast pump flange half 1202a and a second breast pump flange half 1202b that are configured to be coupled together to make an inner volume of the breast pump flange 1202 airtight. In some embodiments, the two halves (1202a, 1202b) of the breast pump flange 1202 can each define one or more concavities and one or more convexities. In some embodiments, the two halves (e.g., 1202a, 1202b) of the breast pump flange 1202 can each define one or more mating surfaces 1204a, 1204b. The breast pump flange halves 1202a, 1202b can further comprise or define one or more coupling mechanisms, which may comprise a plurality of ridges 1206a, 1206b that are configured to be retained within respective of a plurality of channels 1208a, 1208b.

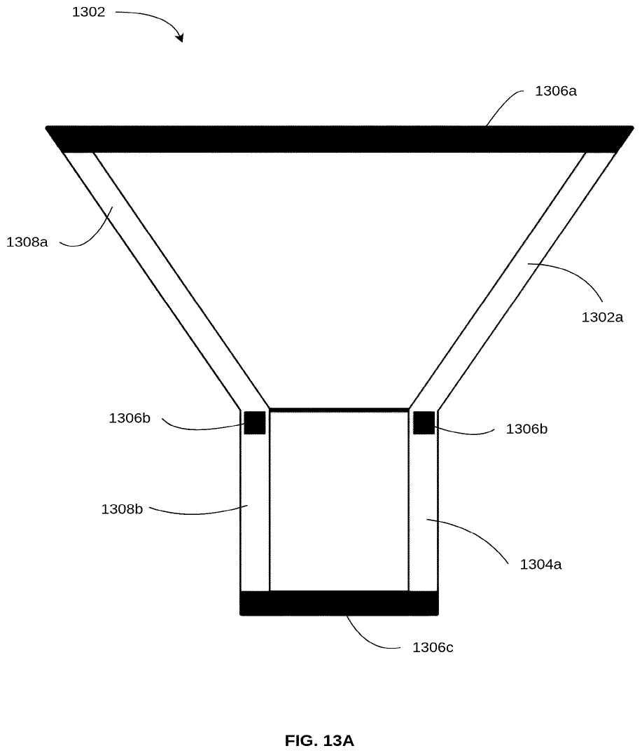

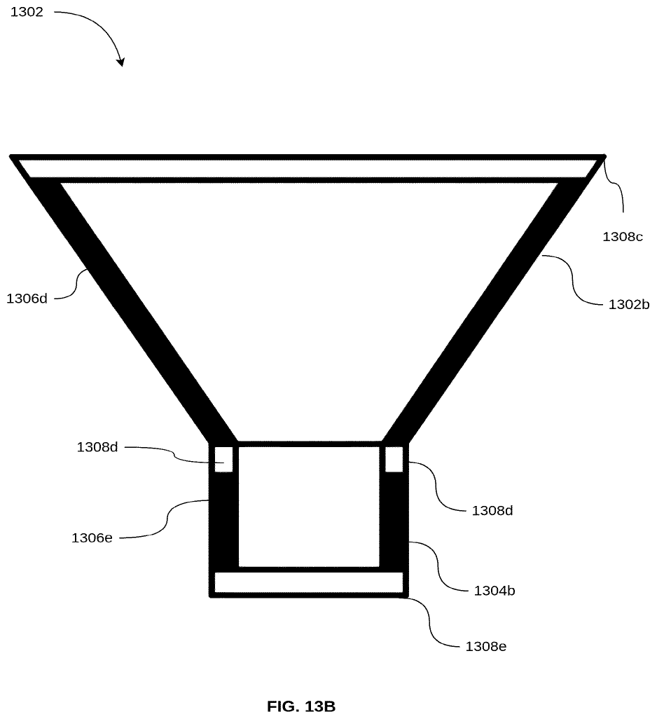

As illustrated in FIGS. 13A and 13B, a breast pump flange 1302 can be provided that comprises a first breast pump flange half 1302a and a second breast pump flange half 1302b that are configured to be coupled together to make an inner volume of the breast pump flange 1302 airtight. In some embodiments, the two halves (1302a, 1302b) of the breast pump flange 1302 can each define one or more concavities and one or more convexities. In some embodiments, the two halves (1302a, 1302b) of the breast pump flange 1302 can each define one or more mating surfaces 1304a, 1304b. The breast pump flange halves 1302a, 1302b can further comprise or define one or more coupling mechanisms, which may comprise a plurality of ridges 1306a, 1306b, 1306c, 1306d, 1306e that are configured to be retained within respective of a plurality of channels 1308a, 1308b, 1308c, 1308d, 1308e.

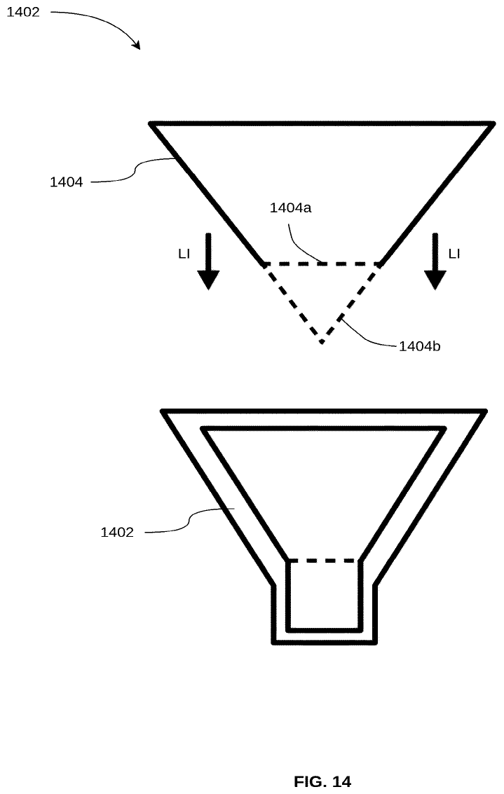

Referring now to FIG. 14, a breast pump flange 1402 is illustrated that is configured to receive a liner 1904 within an inner volume of the breast pump flange 1402. According to some embodiments, the process for disposing the liner 1404 within the breast pump flange 1402 is illustrated as liner insertion, marked LI, in which the liner 1404 is slidably disposed through a top opening of the breast pump flange 1402 until a bottom surface of the liner 1404 comes to rest against one or more inner surfaces within the inner volume of the breast pump flange 1402.

According to some embodiments, the liner 1404 can have a suitable form factor with respect to the form factor of the inner volume of the breast pump flange 1402. For example, in some embodiments, the liner 1404 can have a three-dimensional geometric form factor with a circular opening at a top end and conical shaped wall that tapers smoothly from the circular opening, down to a second circular opening 1404a. In other embodiments, the liner 1404 can have a three-dimensional geometric form factor with a circular opening at a top end and conical shaped wall that tapers smoothly from the circular opening, down to a vertex 1404b.

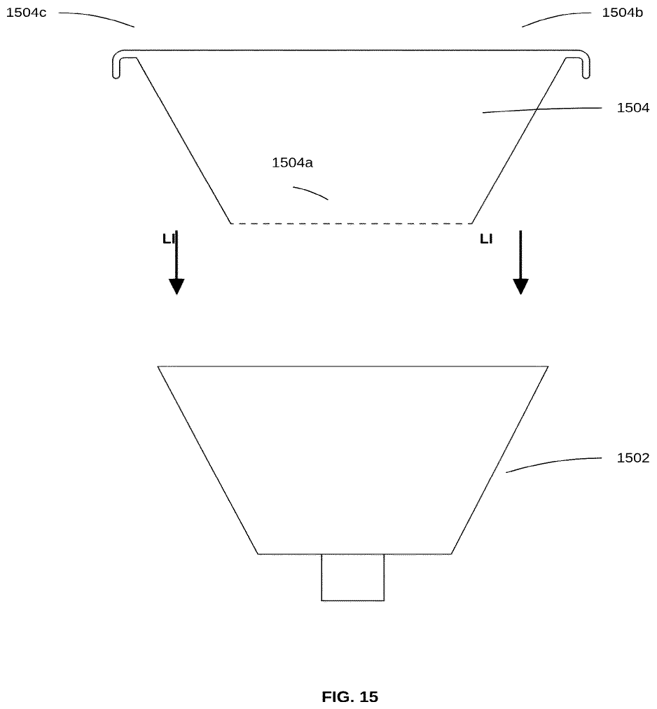

Referring now to FIG. 15, a breast pump flange 1502 is illustrated that is configured to receive a liner 1504 within an inner volume of the breast pump flange 1502. According to some embodiments, the process for disposing the liner 1504 within the breast pump flange 1502 is illustrated as liner insertion, marked LI, in which the liner 1504 is slidably disposed through a top opening of the breast pump flange 1502 until a bottom surface of the liner 1504 comes to rest against one or more inner surfaces within the inner volume of the breast pump flange 1502.

According to some embodiments, the liner 1504 can have a suitable form factor with respect to the form factor of the inner volume of the breast pump flange 1502. For example, in some embodiments, the liner 1504 can have a three-dimensional geometric form factor with a circular opening at a top end and conical shaped wall that tapers smoothly from the circular opening, down to a second circular opening 1504a. In some embodiments, the liner 1504 can further comprise one or more overhanging portions 1504b, 1504c. In some embodiments, the overhanging portions 1504b, 1504c can be configured to be disposed over at least a portion of the top opening of the breast pump flange 2002. According to certain embodiments, the overhanging portions 1504b, 1504c, when disposed over at least a portion of the top opening of the breast pump flange 1502, the overhanging portions 1504b, 1504c of the liner 1504 can further prevent the breast pump flange 1502 from coming into contact with breast tissue or breast milk during use of the breast pump flange 1502 in, e.g., a breast pumping device or system.

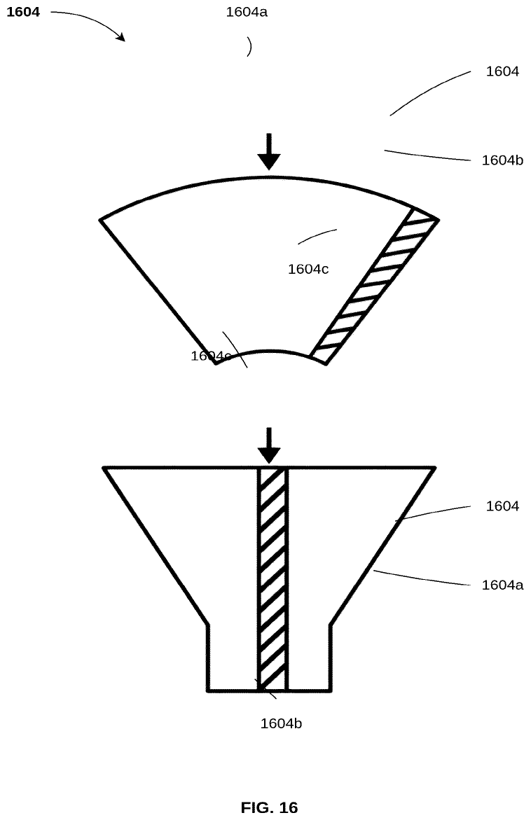

As shown in FIG. 16, a liner 1604 is illustrated from a top view and a side view, respectively. In FIG. 16, the top view of the liner 1604 shows that the liner 1604 comprises a planar portion 1604a, an overlapping portion 1604b, and, optionally, an adhesion portion 1604c. As shown in FIG. 16, when the planar portion 1604a is rotationally or configurationally manipulated into a generally conical shape, the overlapping portion 1604b is configured to overlap an opposite end portion (e.g., end strip) of the planar portion 1604a of the liner 1604. The overlapping portion 1604b of the liner 1604 can be coupled to, joined to, adhered to, or otherwise held in place against the opposite end portion (e.g., end strip) of the planar portion 1604b of the liner 1604 by way of the adhesion portion 1604c.

In some embodiments, the liner 1604 can be provided to a user individually or as part of a kit. In some embodiments, when the liner 1604 is provided to the user, the adhesion portion 1604c of the liner 1604 can be covered by a protective film, or the like. Then, before using the liner 1604, the user can remove the protective film from the adhesion portion 1604c of the liner 1604, roll the planar portion 1604a into a generally conical shape such that the overlapping portion 1604b overlaps an opposite end portion (e.g., end strip) of the planar portion 1604a of the liner 1604, and can press the overlapping portion 1604b of the liner 2104 until the adhesion portion 1604c of the liner 1604 adheres to the opposite end portion (e.g., end strip) of the planar portion 1604b to retain the liner 1604 in the generally conical shape by way of the adhesion portion 1604c.

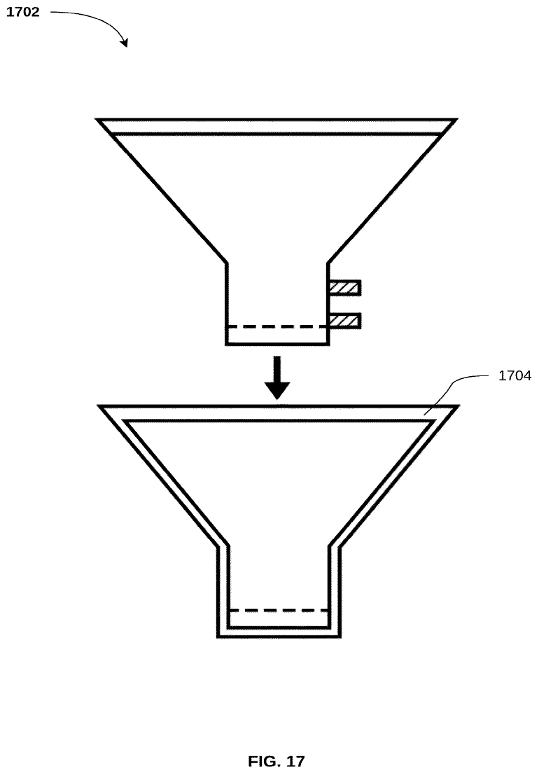

As shown in FIG. 17, a first breast pump flange half 1702 is shown from a side view that is configured to be coupled to or retained in airtight contact with a second breast pump flange half, which may be the same as, or similar to, the first breast pump flange half 1702. As illustrated, a liner 1704 can be provided (illustrated from a top view) that is dimensioned and configured to be disposed on or within the first breast pump flange half 1702. The liner 1704 may be slidably disposed (marked as LI) within or into the inner volume of the first breast pump flange half 2202. The liner 1704 can comprise one or more alignment tabs 1704a, 1704b. In some embodiments, LI can be facilitated by aligning the one or more alignment tabs 1704a, 1704b with one or more registration marks, alignment marks, or features on or of the first breast pump flange half 1702.

In embodiments in which a breast pump flange can be formed by coupling the first breast pump flange half 1702 to a second breast pump flange half (e.g., which may be similar to or identical to the first breast pump flange half 1702), two liners (e.g., such as liner 1704) can be disposed within the two breast pump flange halves (e.g., 1702). Thereafter, when the two breast pump flange halves (e.g., 1702) are coupled together, the two liners (e.g., liner 1704) are disposed within the two portions of the inner volume of the breast pump flange.

In some embodiments, the liner 1704 can be dimensioned such that at least a portion of the liner 1704 extends over one or more edges of the first breast pump flange half 1702.

In some embodiments, the one or more alignment tabs 1704a, 1704b, in addition to being configured to facilitate alignment with one or more registration marks, alignment marks, or features on or of the first breast pump flange half 1702, can function as adhesion strips configured to extend past the end of the first breast pump flange half 1702 and be adhesively coupled to an outer surface of a second breast pump flange half (e.g., which may be similar to or the same as the first breast pump flange half 1702). In some embodiments, the one or more alignment tabs 1704a, 1704b, can function as adhesion strips configured to extend past the end of the first breast pump flange half 1702 and be adhesively coupled to an outer surface of the first breast pump flange half 1702 in order to retain the liner within or one an inner surface of the first breast pump flange half 1702.

In some embodiments, the liner 1704 can be provided to a user individually or as part of a kit. In some embodiments, when the liner 1704 is provided to the user, the one or more alignment tabs 1704a, 1704b of the liner 1704 can be covered by a protective film, or the like. Then, before using the liner 1704, the user can remove the protective film from the one or more alignment tabs 1704a, 1704b of the liner 1704, dispose the liner 1704 onto the inner surfaces of the first breast pump flange half 1702, and press the one or more alignment tabs 1704a, 1704b of the liner 1704 to adhere the liner 1704 to the inner surface of the first breast pump flange half 1702.

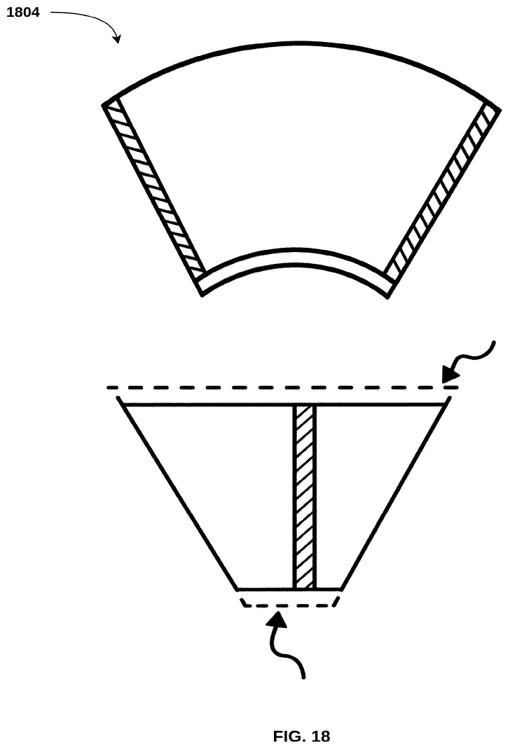

Referring now to FIG. 18, a liner 1804 is illustrated from a top view and a side view. In FIG. 18, the top view of the liner 1804 shows that the liner 1804 comprises an adhesive portion 1804a and an end strip portion 1804b. As shown in FIG. 18, when the liner 1804 is rotationally or configurationally manipulated into a generally conical shape, the adhesive portion 1804a is configured to overlap the end strip portion 1804b of the liner 1804. The adhesive portion 1804a of the liner 1804 can be coupled to, joined to, adhered to, or otherwise held in place against the end strip portion 1804b of the liner 1804 by way of, e.g., an adhesive disposed on the adhesive portion 1804a.

In some embodiments, the liner 1804 can be provided to a user individually or as part of a kit. In some embodiments, when the liner 1804 is provided to the user, the adhesive portion 1804a of the liner 1804 can be covered by a protective film, or the like. Then, before using the liner 1804, the user can remove the protective film from the adhesive portion 1804a of the liner 1804, roll the liner 1804 into a generally conical shape such that the adhesive portion 1804a overlaps the end strip portion 1804b of the liner 1804, and can press the adhesive portion 1804a of t he liner 1804 until the adhesive portion 1804a of the liner 1804 adheres to the end strip portion 1804b of the liner 1804 in the generally conical shape by way of the adhesive portion 1804a.

In some embodiments, the liner 1804 can comprise a first adhesive portion 1804c and a second adhesive portion 1804c. The first adhesive portion 1804c and the second adhesive portion 1804c can be positioned or disposed on an outer surface of the liner 1804. In some embodiments, the internal adhesive portions 1804b, 1804c can be dimensioned and configured to abut an inner surface within the inner volume of a breast pump flange. Like the adhesive portion 1804a, adhesive portions 1804b and 1804c can be covered with a protective film that the user removes after forming the liner 1804 into a generally conical shape but before disposing the liner 1804 into the inner volume of the breast pump flange.

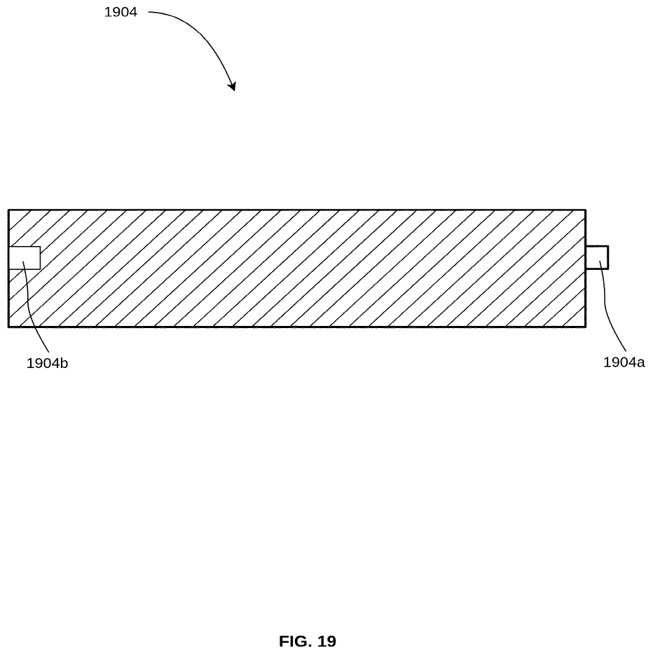

Additionally or alternatively, a liner can be disposed onto or within other components of a breast pump device or system. For example, as illustrated in FIG. 19, a liner 1904 can be provided that has a suitable form factor for the liner 1904 to be disposed within other components of a breast pump device or system. The liner 1904 can have a square or rectangular form factor. In some embodiments, the liner 1904 can comprise one or more alignment tabs 1904a and/or one or more alignment markings 1904b. In some embodiments, an alignment tab 1904a can be positioned at (e.g., extending past) a first end of the liner 1904. In some embodiments, an alignment marking 1904b can be positioned at a second end (e.g., opposite the first end) of the liner 1904. In some embodiments, the liner 1904 may be assembled for use in or with a component of a breast pump device and system by rolling the liner 1904 up from the first end to the second end. In some embodiments, when rolling the liner 1904 up from the first end to the second end, the assembly or reconfiguration of the liner 1904 can be facilitated by aligning the alignment tab 1904a with the alignment marking 1904b to determine that the liner 1904 is properly configured and ready to be disposed within the component of the breast pump device or system.

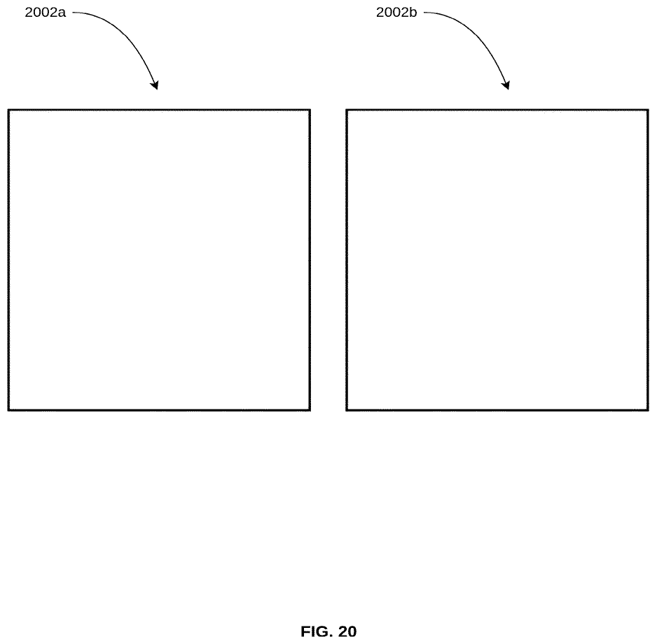

FIG. 20 illustrates a side view of a first and second flange connector half 2002a, 2002b. When the first and second flange connector halves 2002a, 2002b are coupled together, they are configured to form a flange connector.

In some embodiments, the first and second flange connector halves 2002a, 2002b, when coupled together at one or more mating surfaces, can form an inner volume of the flange connector. In some embodiments, the inner volume of the flange connector can be defined by the first and second flange connector halves 2002a, 2002b. In some embodiments, the liner 1904 illustrated in FIG. 19 can be disposed on the inner surfaces of the flange connector as defined by the first and second flange connector halves 2002a, 2002b.

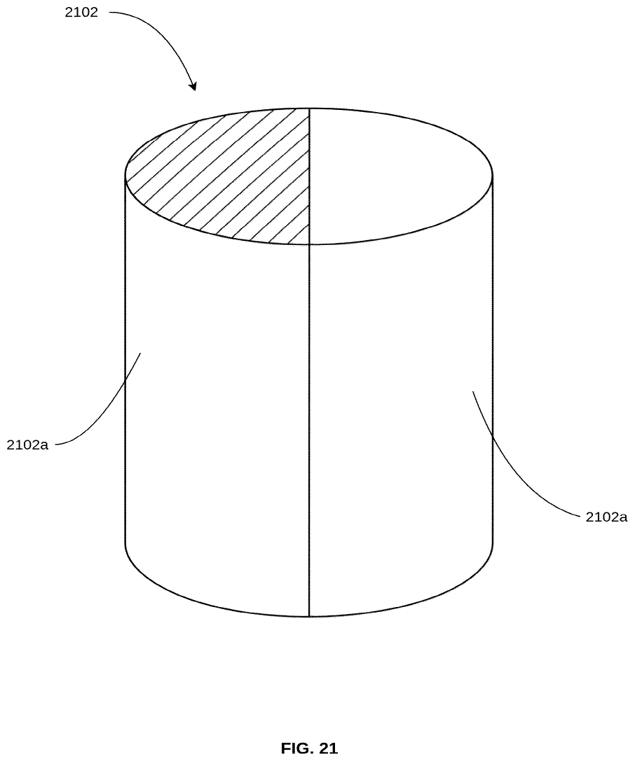

For example, as illustrated in FIG. 21, when first and second flange connector halves 2102a, 2102b are coupled together at one or more mating surfaces, the first and second flange connector halves 2102a, 2102b form a flange connector 2102. The inner surfaces and inner volume within the flange connector 2102 can be dimensioned and configured to receive a liner, such as the liner 1904 illustrated in FIG. 19.

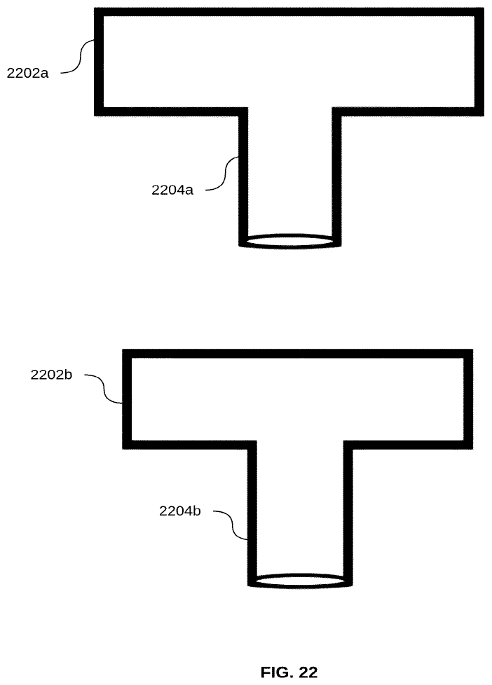

Referring now to FIG. 22, a first duckbill valve half 2202a and a second duckbill valve half 2202b are illustrated from a side view. The first and second duckbill valve halves 2202a, 2202b can, respectively, comprise or define one or more mating surfaces 2204a, 2204b. The first and second duckbill valve halves 2202a, 2202b can be provided to a user as separate pieces and assembled by the user into a duckbill valve. To assemble the first and second duckbill valve halves 2202a, 2202b to form the duckbill valve, the user can align the one or more mating surfaces 2204a of the first duckbill valve half 2202a to the one or more mating surfaces 2204b of the second duckbill valve half 2202b. As with the valve and/or other components, the first and second duckbill valve halves 2202a, 2202b can be coupled together with the mating surfaces 2204a, 2204b abutting using any suitable fastening or connecting means, such as an external fastener or an internal/integral fastener.

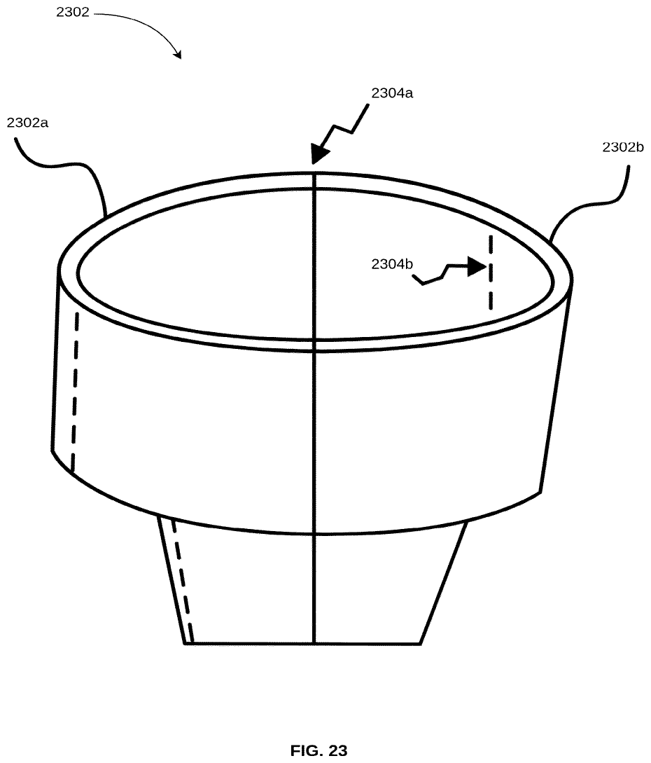

As shown in FIG. 23, a duckbill valve 2302 can be provided that comprises a first duckbill valve half 2302a and a second duckbill valve half 2302b. In some embodiments, the first and second duckbill valve halves 2302a, 2302b can be dimensioned and configured such that the duckbill valve 2302 comprises vertical mating interface 2304a and such that the first and second duckbill valve halves 2302a, 2302b are symmetrical. In some embodiments, the first and second duckbill valve halves 2302a, 2302b can be dimensioned and configured such that the duckbill valve 2302 comprises vertical mating interface 2304b and such that the first and second duckbill valve halves 2302a, 2302b are asymmetrical.

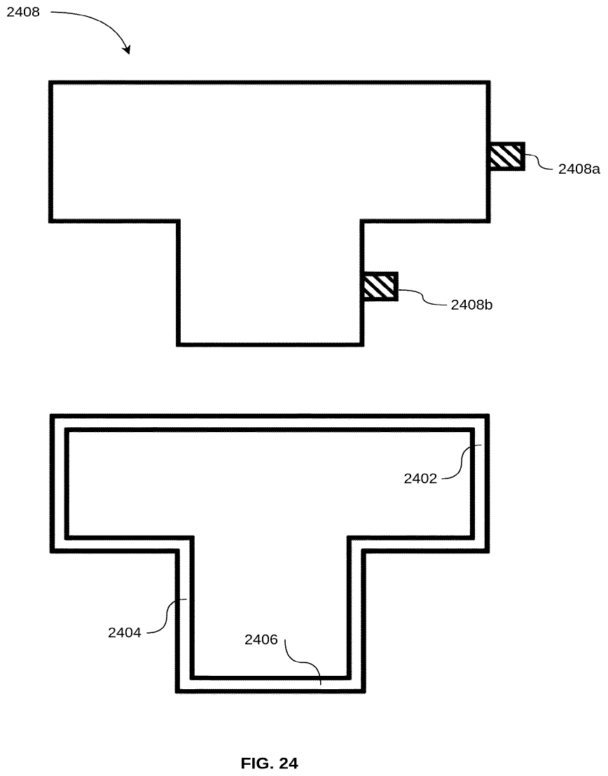

Referring now to FIG. 24, a duckbill valve half 2402 can be provided that comprises one or more mating surfaces 2404 and one or more valve apertures 2406. The duckbill valve half 2402 can comprise one or more convexities and/or one or more concavities. In some embodiments, the duckbill valve half 2402 can comprise an inner surface that is configured to receive a liner 2408. The liner 2408 can be dimensioned and configured to be disposed/retained on the inner surface of the duckbill valve half 2402. The liner 2408 can comprise one or more alignment tabs 2408a, 2408b. The one or more alignment tabs 2408a, 2408b can be used to align the liner 2408 within an inner volume of the duckbill valve half 2402 or to align the liner 2408 on the inner surface of the duckbill valve half 2402.

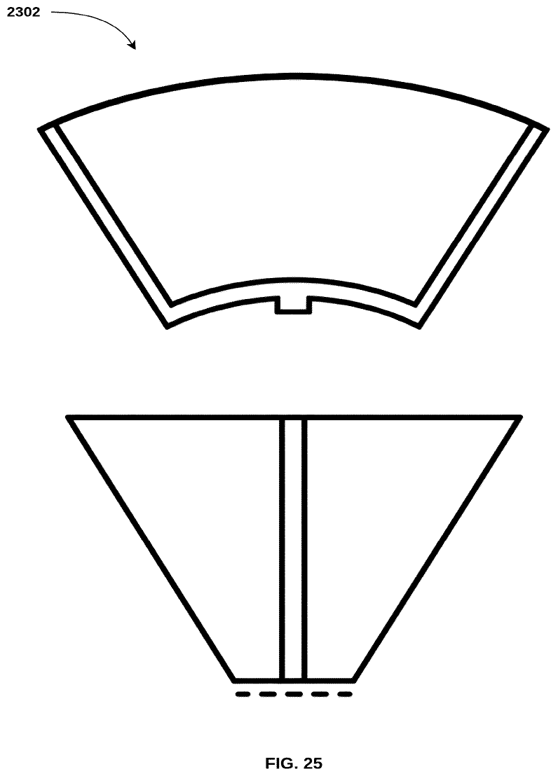

Referring now to FIG. 25, a backflow preventor half 2502 can be provided that comprises one or more mating surfaces 2504. The mating surfaces 2504 can be configured to allow the backflow preventor half 2502 to be coupled to one or more other mating surfaces of a second backflow preventor half (e.g., which may be similar to or the same as the backflow preventor half 2502). The duckbill valve half 2502 can comprise one or more convexities and/or one or more concavities. In some embodiments, the duckbill valve half 2502 can comprise an inner surface that is configured to receive a liner 2508. The liner 2508 can be dimensioned and configured to be disposed/retained on the inner surface of the duckbill valve half 2502. The liner 2508 can comprise a deformable portion 2508a. The liner 2508 can further comprise a bottom opening 2508b.

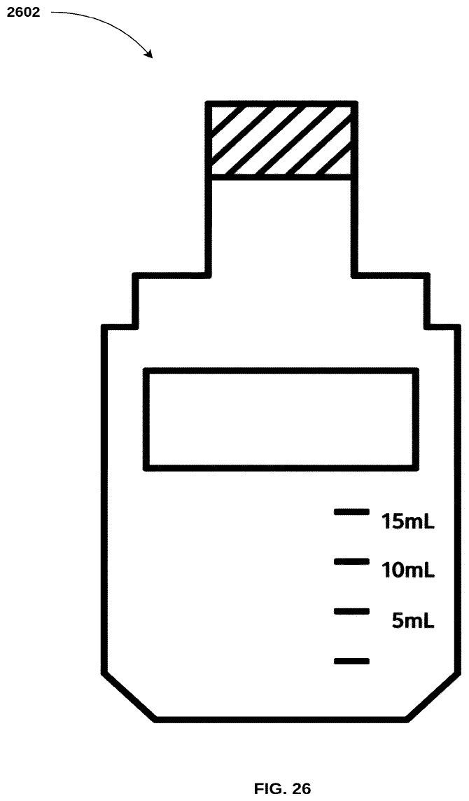

Referring now to FIG. 26, a lactation fluid collection container 2602 can be provided that is dimensioned and configured to be removably coupled to other components of a breast pump system or device. For example, the lactation fluid collection container 2602 can comprise a collection throat 2602a fluidically coupled to a collection volume 2602b of the lactation fluid collection container 2602. The collection throat 2602a is configured to receive therewithin a portion of a duckbill valve, such as 2202, 2302, 2402. In some embodiments, when a duckbill valve is disposed within the collection throat 2602a of the lactation fluid collection container 2602, the lactation fluid collection container 2602 can be retained in place (e.g., the duckbill valve retained in place within the collection throat 2602a of the lactation fluid collection container 2602), by any suitable means. According to some embodiments, the lactation fluid collection container 2602 can further comprise one or more adhesion portions 2602c. The one or more adhesion portions 2602c can be positioned on or within an inner surface within the collection throat 2602a. The one or more adhesion portions 2602c positioned on or within the inner surface within the collection throat 2602a can be configured to adhere to an outside of the duckbill valve (e.g., 2202, 2302, 2402) during use of the beast pumping system or device.

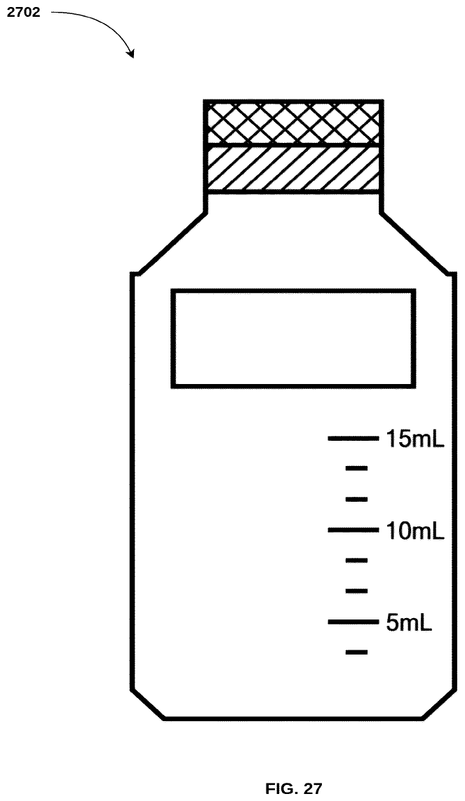

Referring now to FIG. 27, a lactation fluid collection container 2702 can be provided that is dimensioned and configured to be removably coupled to other components of a breast pump system or device. For example, the lactation fluid collection container 2702 can comprise a collection throat 2702a fluidically coupled to a collection volume 2702b of the lactation fluid collection container 2702. The collection throat 2702a is configured to receive therewithin a portion of a duckbill valve, such as 2202, 2302, 2402. In some embodiments, when a duckbill valve is disposed within the collection throat 2702a of the lactation fluid collection container 2702, the lactation fluid collection container 2702 can be retained in place (e.g., the duckbill valve retained in place within the collection throat 2702a of the lactation fluid collection container 2702), by any suitable means. According to some embodiments, the lactation fluid collection container 2702 can further comprise one or more first adhesion portions 2702c. The one or more first adhesion portions 2702c can be positioned on or within an inner surface within the collection throat 2702a. The one or more first adhesion portions 2702c positioned on or within the inner surface within the collection throat 2702a can be configured to adhere to an outside of the duckbill valve (e.g., 2202, 2302, 2402) during use of the beast pumping system or device.

In some embodiments, the lactation fluid collection container 2702 can further comprise one or more second adhesion portions 2702d. In some embodiments, the one or more second adhesion portions 2702d can be positioned on or about an outer surface of the collection throat 2702a. In some embodiments, the one or more second adhesion portions 2702d can be configured to adhere to one or more other portions of an outer surface of the lactation fluid collection container 3202. For example, after using the breast pump device or system, the lactation fluid collection container 3202 can be sealed or partially sealed using the one or more second adhesion portions 2702d by folding over the collection throat 2702a until the one or more second adhesion portions 2702d on the outside of the collection throat 2702a to adhere to the outer surface of the collection volume 2702b of the lactation fluid collection container 2702.

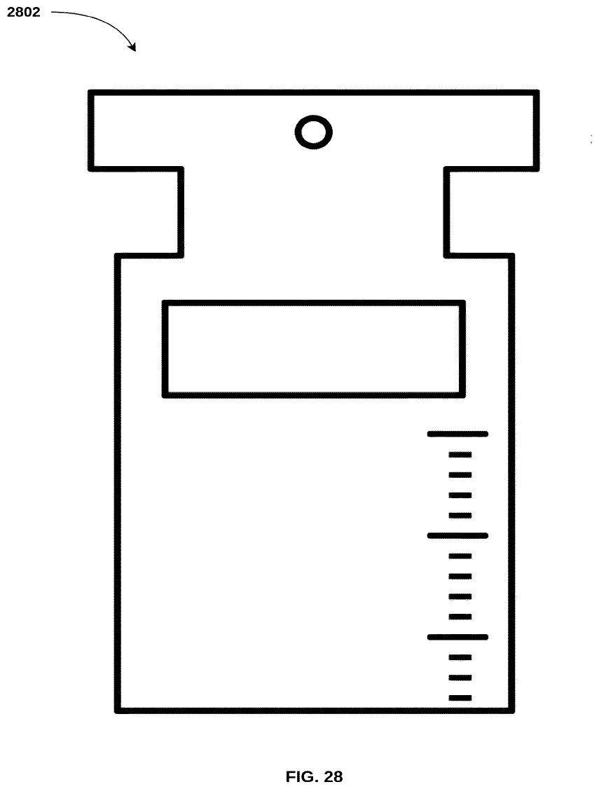

Referring now to FIG. 28, a lactation fluid collection container 2802 can be provided that is dimensioned and configured to be removably coupled to other components of a breast pump system or device. For example, the lactation fluid collection container 2802 can comprise a collection throat 2802a fluidically coupled to a collection volume 2802b of the lactation fluid collection container 2802. The collection throat 2802a is configured to receive therewithin a portion of a duckbill valve, such as 2202, 2302, 2402. In some embodiments, when a duckbill valve is disposed within the collection throat 3302a of the lactation fluid collection container 2802, the lactation fluid collection container 2802 can be retained in place (e.g., the duckbill valve retained in place within the collection throat 2802a of the lactation fluid collection container 2802), by any suitable means. According to some embodiments, the lactation fluid collection container 2802 can further comprise one or more retention elements 2802c. The one or more retention elements 2802c can be positioned on or within the collection throat 2802a. The one or more retention elements 2802c positioned on or within the collection throat 2802a can be configured to be coupled to or about the duckbill valve (e.g., 2202, 2302, 2402) during use of the beast pumping system or device.

In some embodiments, the one or more retention elements 2802c can comprise one or more apertures (e.g., grommeted apertures) through the collection throat 2802a of the lactation fluid collection container 2802. In some embodiments, the lactation fluid collection container 2802 can be retained within place about the duckbill valve or another portion or component of the breast pumping device or system using one or more hangers that are disposed or partially disposed through the apertures through the collection throat 2802a that define the one or more retention elements 2802c.

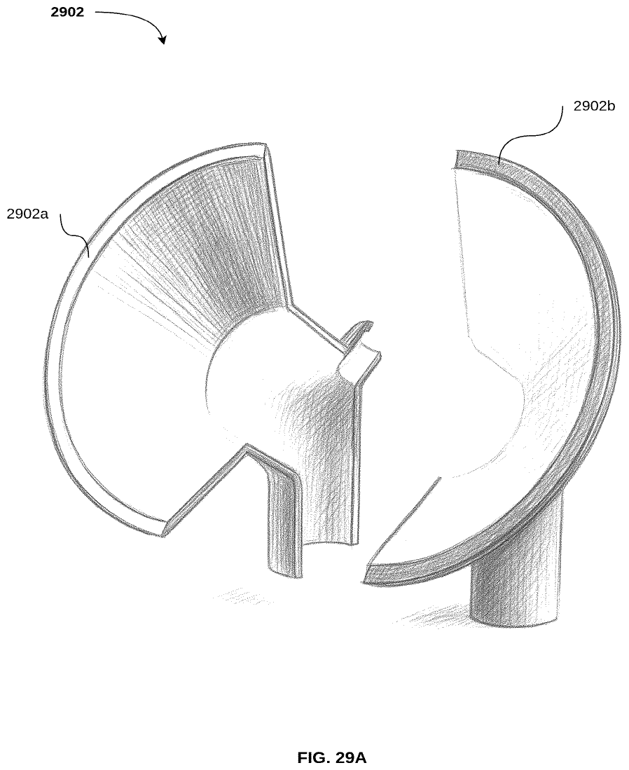

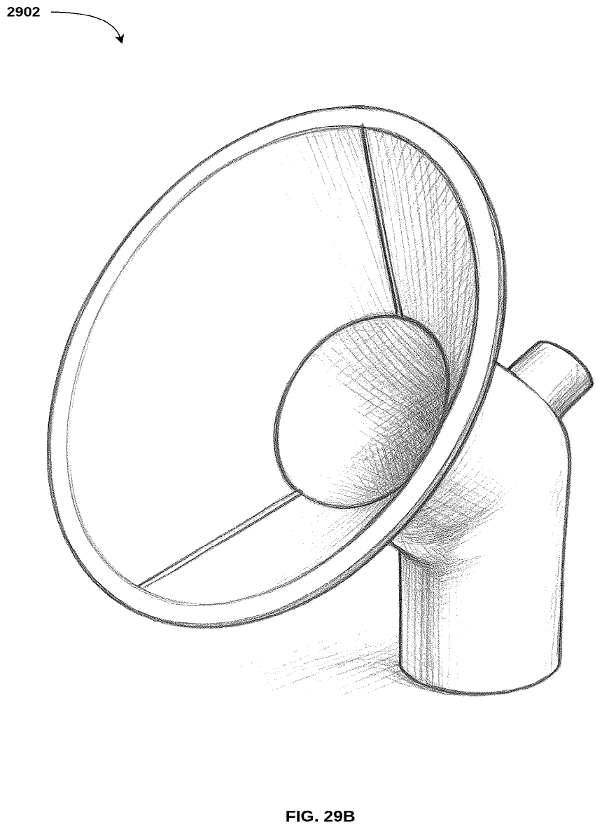

Referring now to FIGS. 29A and 29B, a breast pump flange 2902 is illustrated from a top view that is modular in nature. The breast pump flange 2902 comprises a first flange half 2902a and a second flange half 2902b. The two flange halves 2902a, 2902b can be dimensioned and configured to be joined together or coupled together. The two flange halves 2902a, 2902b can be provided to a user as part of a kit and assembled by the user before use. As described above, the two flange halves 2902a, 2902b can be coupled together using any suitable external coupling or fastening means, any suitable internal or integrated coupling or fastening means, or any suitable combination thereof.

In some embodiments, the first breast pump flange half 2902a and the second breast pump flange half 2902b can be configured to be coupled together to make an inner volume of the breast pump flange 2902 airtight. In some embodiments, the two halves (2902a, 2902b) of the breast pump flange 2902 can each define one or more concavities and one or more convexities. In some embodiments, the two halves (2902a, 2902b) of the breast pump flange 2902 can each define one or more mating surfaces. For example, the first breast pump flange half 2902a can comprise a first mating surface 2904a and the second breast pump flange half 2902b can comprise a second mating surface 2904b. In some embodiments, the first breast pump flange half 2902a and/or the second breast pump flange half 2902b can comprise a plurality of mating surfaces.

In some embodiments, the first breast pump flange half 2902a can comprise a first flange connector 2902c and the second breast pump flange half 2902b can comprise a second flange connector 2902d. In some embodiments, the first breast pump flange half 2902a can further comprise a first backflow preventor connector 2902e and the second breast pump flange half 2902b can further comprise a second backflow preventor connector 2902f.

In some embodiments, the first breast pump flange half 2902a can be joined with/coupled to the second breast pump flange half 2902b via the first and second mating surfaces 2904a, 2904b.

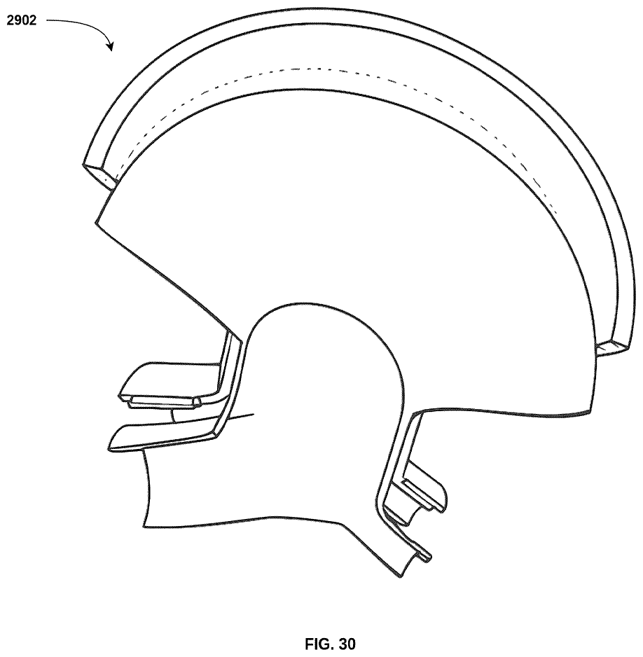

Referring now to FIG. 30, a breast pump flange 3002 is illustrated from a top view that is modular in nature. The breast pump flange 3002 comprises a first flange half 3002a and a second flange half 3002b. The two flange halves 3002a, 3002b can be dimensioned and configured to be joined together or coupled together. The two flange halves 3002a, 3002b can be provided to a user as part of a kit and assembled by the user before use. As described above, the two flange halves 3002a, 3002b can be coupled together using any suitable external coupling or fastening means, any suitable internal or integrated coupling or fastening means, or any suitable combination thereof.

In some embodiments, the first breast pump flange half 3002a and the second breast pump flange half 3002b can be configured to be coupled together to make an inner volume of the breast pump flange 3002 airtight. In some embodiments, the two halves (3002a, 3002b) of the breast pump flange 3002 can each define one or more concavities and one or more convexities. In some embodiments, the two halves (3002a, 3002b) of the breast pump flange 3002 can each define one or more mating surfaces. For example, the first breast pump flange half 3002a can comprise a first mating surface 3004a and the second breast pump flange half 3002b can comprise a second mating surface 3004b. In some embodiments, the first breast pump flange half 3002a and/or the second breast pump flange half 3002b can comprise a plurality of mating surfaces.

In some embodiments, the first breast pump flange half 3002a can comprise a first flange connector 3002c and the second breast pump flange half 3002b can comprise a second flange connector 3002d. In some embodiments, the first breast pump flange half 3002a can further comprise a first backflow preventor connector 3002e and the second breast pump flange half 3002b can further comprise a second backflow preventor connector 3002f.

In some embodiments, the first breast pump flange half 3002a can be joined with/coupled to the second breast pump flange half 3002b via the first and second mating surfaces 3004a, 3004b.

In some embodiments, the first breast pump flange half 3002a can be dimensioned and configured to be nested within or partially within the second breast pump flange half 3002b for storage, transportation, kit assembly, etc.

In any of the embodiments disclosed herein, any of the components can be provided in a partially coupled or partially assembled state. For example, a breast pump flange can be provided in a clamshell arrangement or configuration. A breast pump flange can be provided that comprises a first half that is coupled/joined to a second half at, e.g., a hinge, a joint, a pivot, an axis, a fulcrum, an axle, a swivel, a spindle, or the like. In some embodiments, other components that can be provided in a partially coupled or partially joined state can include a breast pump flange connector, a backflow preventor, a backflow preventor body, a backflow preventor connector, a valve, a duckbill valve, a valve connector, a duckbill valve connector, a collection container, one or more liners for a breast pump flange, one or more liners for any other components or subcomponents disclosed herein, and/or the like.

In any of the embodiments disclosed herein, any of the components disclosed herein can be dimensioned and configured to receive one or more liners to cover or partially cover one or more inner surfaces of the component. In some embodiments, the liner can comprise one or multiple layers. For example, a liner can comprise a plurality of layers that are stratified, or otherwise arranged into a plurality of liner layers. In some embodiments, the plurality of layers of the liner(s) can be configured to be removed in a layer-by-layer manner. In some embodiments, after a first use of a component comprising a liner disposed on inside surfaces of the component, an uppermost/outside layer of the liner can be removed while leaving the remainder of the layers of the liner(s), thereby leaving one or more remaining layers of the liner(s) disposed on the inside surface(s) of the component.

The manufacturing, storage, shipping, provisioning, kit assembly, kit disassembly, and/or use of any of the above components (e.g., 902, 1002, 1102, 1202, 1302, 1402, 1404, 1502, 1504, 1604, 1702, 1704, 1804, 1904, 2002, 2102, 2202, 2304, 2402, 2408, 2502, 2508, 2602, 2702, 2802, 2902, or 3002) can be performed, such as according to the below non-limiting embodiments.

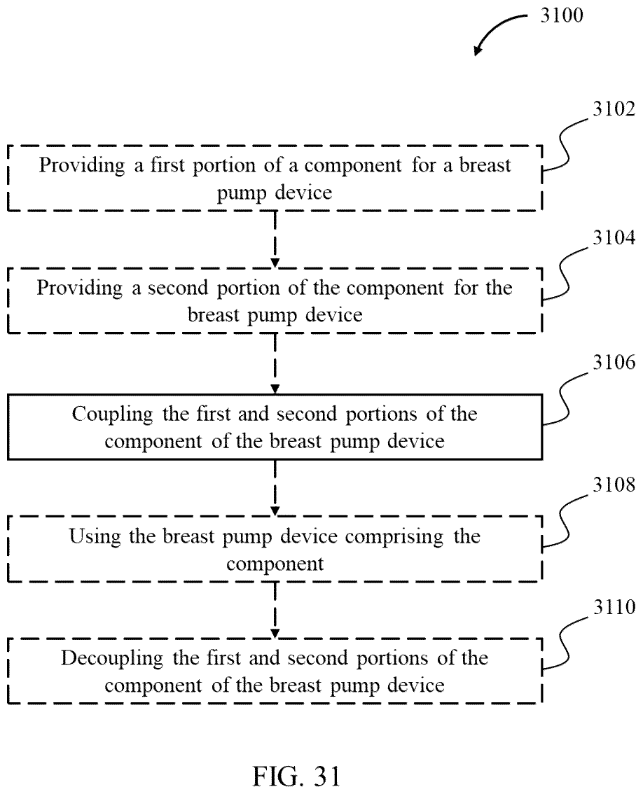

FIG. 31 is a block flow diagram illustrating a method 3100 that, optionally, comprises providing a first portion of a component for a breast pump device, at 3102. The method 3100 can, optionally, further comprise providing a second portion of the component for the breast pump device, at 3104. The method 3100 can comprise coupling the first and second portions of the component of the breast pump device, at 3106. The method 3100 can, optionally, further comprise using the breast pump device comprising the component, at 3108. The method 3100 can, optionally, further comprise decoupling the first and second portions of the component of the breast pump device, at 3110.

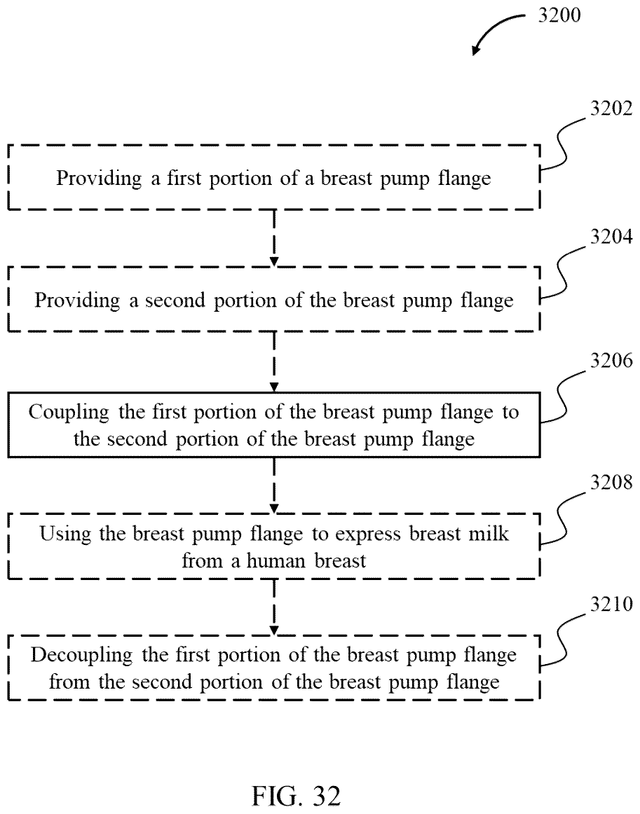

FIG. 32 is a block flow diagram illustrating a method 3200 that, optionally, comprises providing a first portion of a breast pump flange, at 3202. The method 3200 can, optionally, further comprise providing a second portion of the breast pump flange, at 3204. The method 3200 comprises coupling the first portion of the breast pump flange to the second portion of the breast pump flange, at 3206. The method 3200 can, optionally, further comprise using the breast pump flange to express breast milk from a human breast, at 3208. The method 3200 can, optionally, further comprise decoupling the first portion of the breast pump flange from the second portion of the breast pump flange, at 3210.

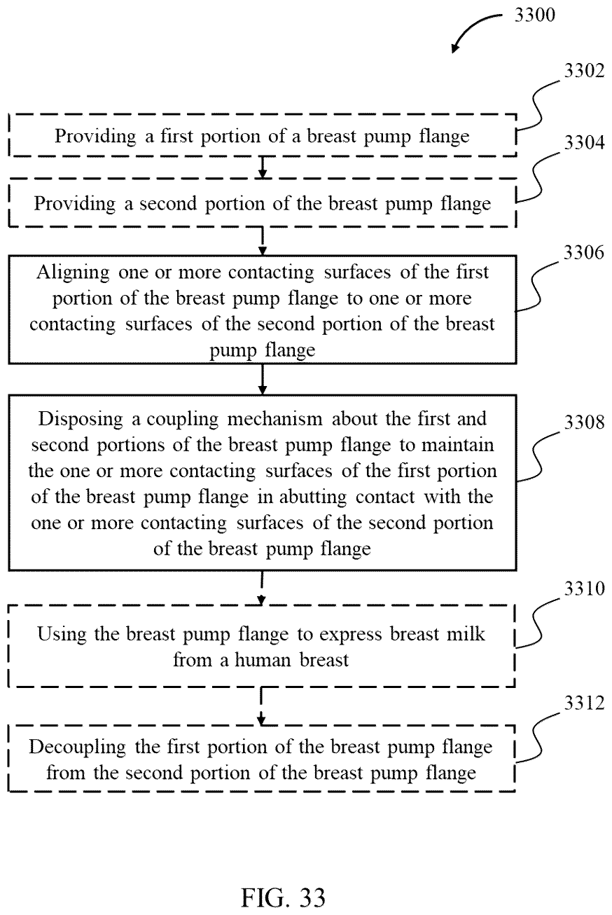

FIG. 33 is a block flow diagram illustrating a method 3300 that, optionally, comprises providing a first portion of a breast pump flange, at 3302. The method 3300, optionally, further comprises providing a second portion of the breast pump flange, at 3304. The method 3300 comprises aligning one or more contacting surfaces of the first portion of the breast pump flange to one or more contacting surfaces of the second portion of the breast pump flange, at 3306. The method 3300 can further comprise disposing a coupling mechanism about the first and second portions of the breast pump flange to maintain the one or more contacting surfaces of the first portion of the breast pump flange in abutting contact with the one or more contacting surfaces of the second portion of the breast pump flange, at 3308. The method 3300 can, optionally, further comprise Using the breast pump flange to express breast milk from a human breast, at 3310. The method 3300 can, optionally, further comprise decoupling the first portion of the breast pump flange from the second portion of the breast pump flange, at 3312.

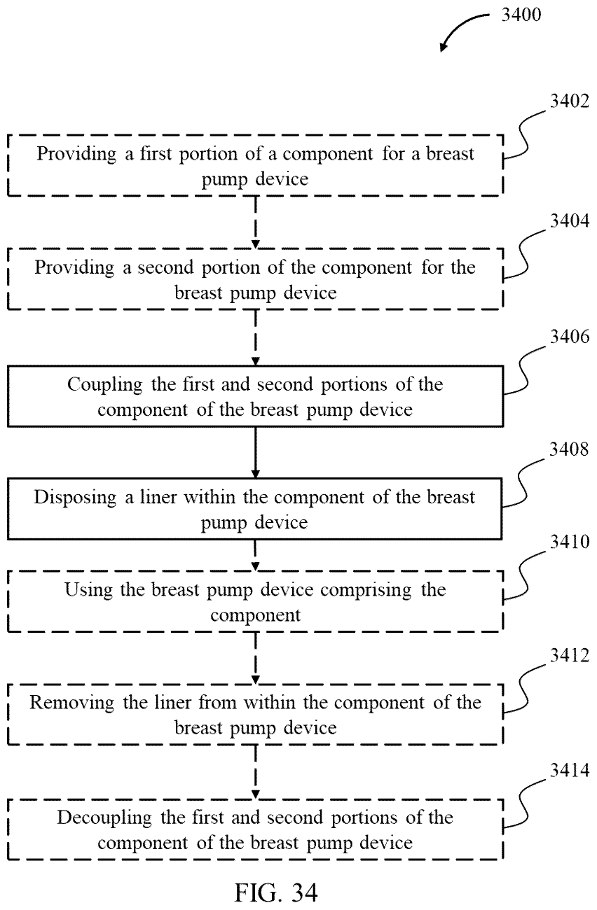

FIG. 34 is a block flow diagram illustrating a method 3400 that, optionally, comprises providing a first portion of a component for a breast pump device, at 3402. The method 3400 can optionally, further comprise providing a second portion of the component for the breast pump device, at 3404. The method 3400 comprises coupling the first and second portions of the component of the breast pump device, at 3406. The method 3400 further comprises disposing a liner within the component of the breast pump device, at 3408. The method 3400 can, optionally, further comprise using the breast pump device comprising the component, at 3410. The method 3400 can, optionally, further comprise removing the liner from within the component of the breast pump device, at 3412. The method 3400 can, optionally, further comprise decoupling the first and second portions of the component of the breast pump device, at 3414.

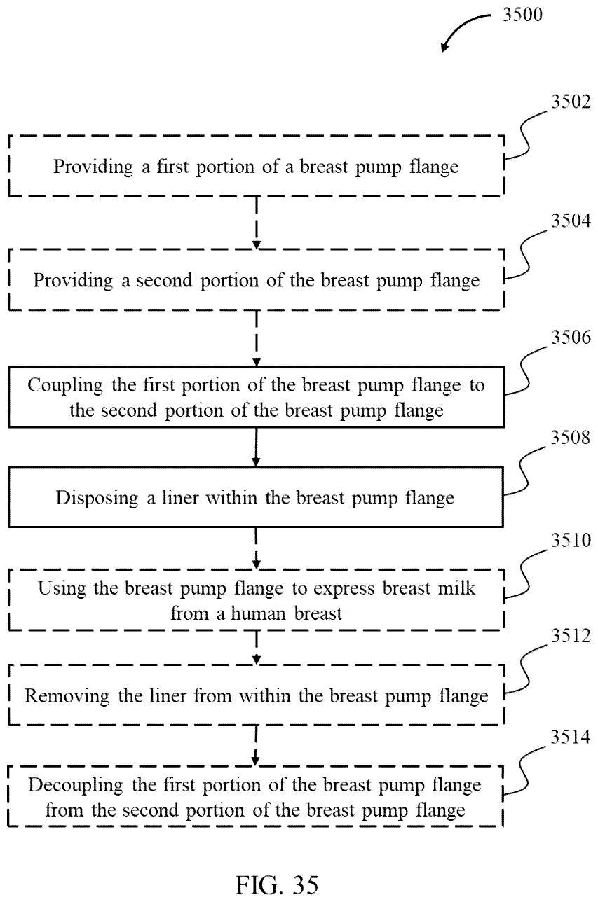

FIG. 35 is a block flow diagram illustrating a method 3500 that, optionally, comprises providing a first portion of a breast pump flange, at 3502. The method 3500 can, optionally, further comprise providing a second portion of the breast pump flange, at 3504. The method 3500 comprises coupling the first portion of the breast pump flange to the second portion of the breast pump flange, at 3506. The method 3500 further comprises disposing a liner within the breast pump flange, at 3508. The method 3500 can, optionally, further comprise using the breast pump flange to express breast milk from a human breast, at 3510. The method 3500 can, optionally, further comprise removing the liner from within the breast pump flange, at 3512. The method 3500 can, optionally, further comprise decoupling the first portion of the breast pump flange from the second portion of the breast pump flange, at 3514.

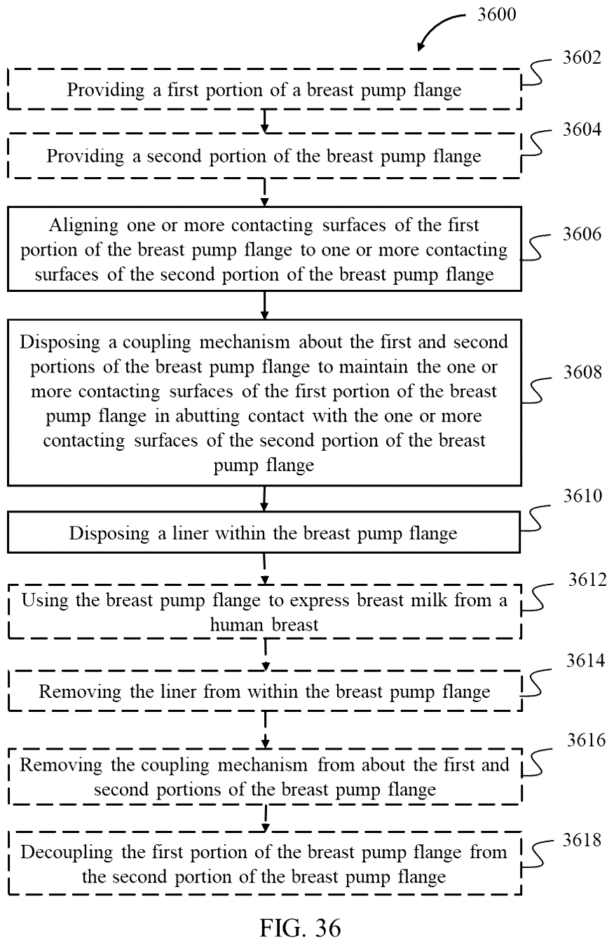

FIG. 36 is a block flow diagram illustrating a method 3600 that, optionally, comprises providing a first portion of a breast pump flange, at 3602. The method 3600 can, optionally, further comprise providing a second portion of the breast pump flange, at 3604. The method 3600 comprises aligning one or more contacting surfaces of the first portion of the breast pump flange to one or more contacting surfaces of the second portion of the breast pump flange, at 3606. The method 3600 further comprises disposing a coupling mechanism about the first and second portions of the breast pump flange to maintain the one or more contacting surfaces of the first portion of the breast pump flange in abutting contact with the one or more contacting surfaces of the second portion of the breast pump flange, at 3608. The method 3600 further comprises disposing a liner within the breast pump flange, at 3610. The method 3600 can, optionally, further comprise using the breast pump flange to express breast milk from a human breast, at 3612. The method 3600 can, optionally, further comprise removing the liner from within the breast pump flange, at 3614. The method 3600 can, optionally, further comprise removing the coupling mechanism from about the first and second portions of the breast pump flange, at 3616. The method 3600 can, optionally, further comprise decoupling the first portion of the breast pump flange from the second portion of the breast pump flange, at 3618.

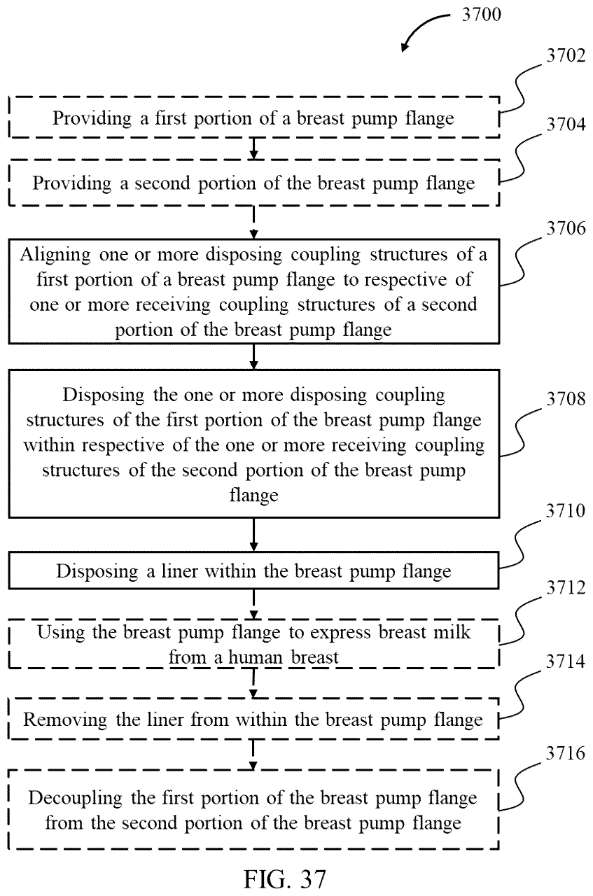

FIG. 37 is a block flow diagram illustrating a method 3700 that, optionally, comprises providing a first portion of a breast pump flange, at 3702. The method 3700 can, optionally, further comprise providing a second portion of the breast pump flange, at 3704. The method 3700 comprises aligning one or more disposing coupling structures of a first portion of a breast pump flange to respective of one or more receiving coupling structures of a second portion of the breast pump flange, at 3706. The method 3700 further comprises disposing the one or more disposing coupling structures of the first portion of the breast pump flange within respective of the one or more receiving coupling structures of the second portion of the breast pump flange, at 3708. The method 3700 further comprises disposing a liner within the breast pump flange, at 3710. The method 3700 can, optionally, further comprise using the breast pump flange to express breast milk from a human breast, at 3712. The method 3700 can, optionally, further comprise removing the liner from within the breast pump flange, at 3714. The method 3700 can, optionally, further comprise decoupling the first portion of the breast pump flange from the second portion of the breast pump flange, at 3716.

According to various embodiments described herein, one or more of these methods (e.g., 3600-5100) can be carried out at least in part using one or more of the of the systems (e.g., 100, 200), devices (e.g., 300), kits (e.g., 400), or components (e.g., 505, 614, 716, 818, 902, 1002, 1004, 1102, 1104, 1202, 1204, 1302, 1304, 1402, 1404, 1502, 1504, 1600, 1702, 1704, 1802, 1804, 1904, 2002, 2102, 2202, 2204, 2302, 2402, 2404, 2502, 2504, 2602, 2702, 2802, 2902, and 3002) described herein.

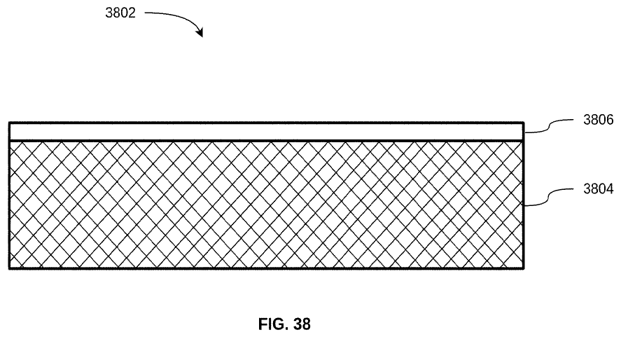

Referring now to FIGS. 38-48, various embodiments of breast pump components formed from laminate materials comprising compressed fiber materials and barrier layers are illustrated and described. These laminate structures provide a combination of structural rigidity, reduced weight, liquid impermeability, and mechanical compliance that enables the use of biodegradable and compostable materials in breast pump applications where such materials would otherwise be unsuitable.

FIG. 38 illustrates a side profile view of a planar laminate material 3802 comprising a compressed fiber material layer 3804 and a barrier layer 3806 disposed on at least one surface of the compressed fiber material layer 3804. The compressed fiber material layer 3804 forms the structural layer of the laminate and may comprise one or more cellulosic fiber materials including, but not limited to, cellulose fibers, wood pulp fibers, bamboo fibers, bagasse fibers, hemp fibers, flax fibers, cotton fibers, recycled paper fibers, wheat straw fibers, or combinations thereof. The compressed fiber material layer 3804 may be formed through wet molded fiber forming, dry molded fiber forming, thermoforming, compression molding, or hot pressing of fibrous material. In some embodiments, the compressed fiber material layer 3804 comprises a blend of two or more fibrous materials selected to balance stiffness and toughness. The compressed fiber material layer 3804 may have a density in a range of 0.3 to 1.2 g/cm³, 0.4 to 0.8 g/cm³, or 0.5 to 0.7 g/cm³, and may have a thickness in a range of 0.3 mm to 5 mm, 0.5 mm to 3 mm, or 1 mm to 2 mm.

The barrier layer 3806 disposed on the compressed fiber material layer 3804 provides a liquid-impermeable, food-contact-compatible, and mechanically compliant surface suitable for contact with lactation fluid. The barrier layer 3806 may comprise one or more of polyethylene, polypropylene, ethylene-vinyl acetate (EVA), polyurethane, polycaprolactone, polylactic acid (PLA), polyhydroxyalkanoates (PHA), polybutylene adipate terephthalate (PBAT), or combinations thereof. In some embodiments, the barrier layer 3806 comprises a multilayer film including at least one elastomeric layer and at least one barrier layer. The barrier layer 3806 may have a thickness in a range of 5 μm to 300 μm, 10 μm to 150 μm, or 25 μm to 100 μm, and may have an elongation at break of at least 100%, at least 200%, or at least 400%.

The laminate material may be formed by laminating the barrier layer 3806 onto the compressed fiber material layer 3804 using heat, pressure, adhesives, extrusion coating, or combinations thereof. In some embodiments, the barrier layer 3806 is formed by extrusion, blown-film extrusion, cast-film extrusion, solution casting, melt casting, calendaring, or extrusion coating. The barrier layer 3806 may be applied to the compressed fiber material layer 3804 through heat and pressure lamination, adhesive lamination, extrusion coating directly onto the substrate, spray coating, dip coating in a polymeric dispersion or solution, or roll-to-roll coating or lamination processes.

The compressed fiber material layer 3804 by itself may be permeable to lactation fluid and mechanically unsuitable for breast pump use due to its tendency to absorb liquids, soften when wet, and lack sufficient elastic recovery under cyclic vacuum loading. However, laminating the barrier layer 3806 onto the compressed fiber material layer 3804 produces a composite structure that simultaneously meets liquid-impermeability requirements for milk contact and elastic flexure requirements for repeated partial-vacuum operation of a breast pump device. The barrier layer 3806 can be engineered, dimensioned, and/or configured to reduce liquid permeation through the laminate material (reducing or eliminating liquid permeation through the compressed fiber material layer 3804) by at least an order of magnitude relative to the compressed fiber material layer 3804 alone, and may reduce liquid absorption into the compressed fiber material layer 3804 by at least 90%, at least 95%, or at least 99% relative to uncoated fiber material (e.g., the compressed fiber material layer 3804 alone).

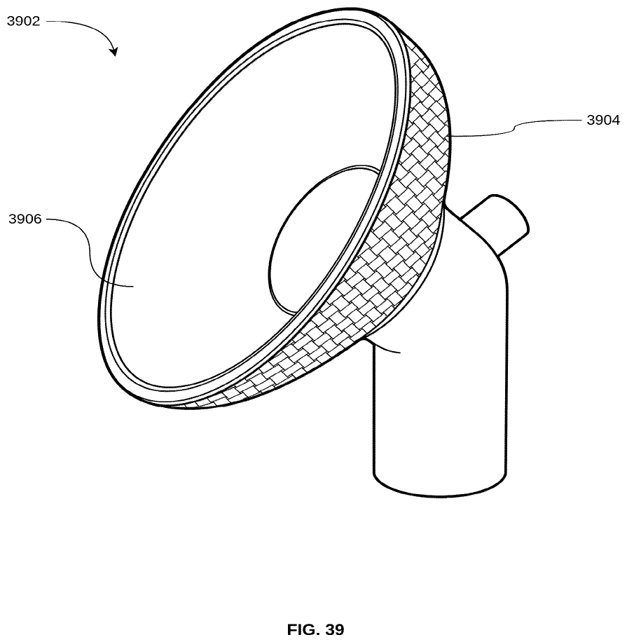

FIG. 39 illustrates a perspective view of a breast shield 3902 formed from a laminate material (e.g., 3802). The breast shield 3902 can comprise a funnel-shaped flange with a wide circular opening that tapers downward to a narrower cylindrical connector base. The outer surface 3904 of the flange can comprise or be formed from a compressed fiber material (e.g., 3804) that forms a structural layer of the laminate, providing rigidity and shape retention while reducing the overall mass of the component. An inner surface 3906 of the flange is defined by a barrier layer (e.g., 3806), which serves as the lactation fluid contact surface during breast pumping. This configuration demonstrates how the planar laminate material can be formed into a three-dimensional breast pump component while maintaining the layered structure of the compressed fiber material (e.g., 3804) and barrier (e.g., 3806).

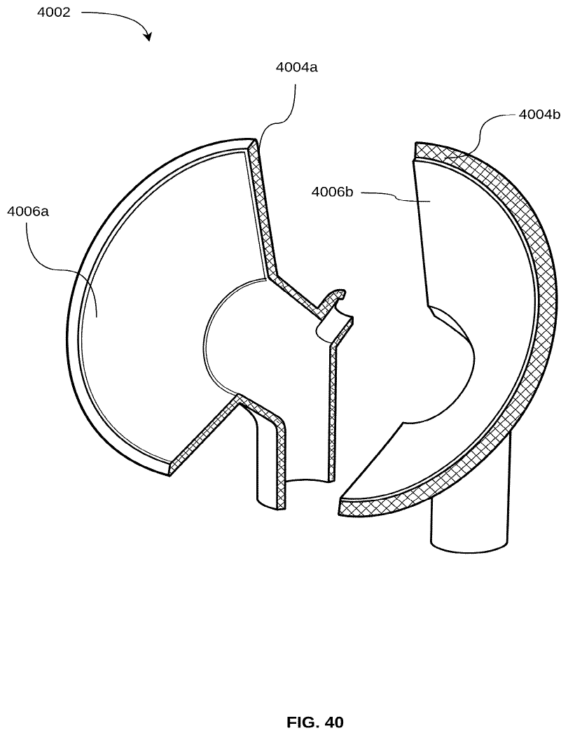

FIG. 40 illustrates an exploded perspective view of a modular breast shield 4002 formed from the laminate material, showing a two-part breast shield assembly in an exploded configuration. The breast shield 4002 comprises a first portion and a second portion that are configured to be coupled together to form the breast pump component. Each of the first portion and the second portion is formed from the laminate material comprising a compressed fiber material layer (e.g., 3804) and a barrier layer (e.g., 3806) disposed on at least one surface of the compressed fiber material. In an assembled configuration, the barrier layer of each of the first portion and the second portion defines at least one internal surface of the breast pump component that is configured for contact with lactation fluid. The compressed fiber material provides structural support and reduced weight while the barrier layer provides regulatory compatibility and mechanical compliance.

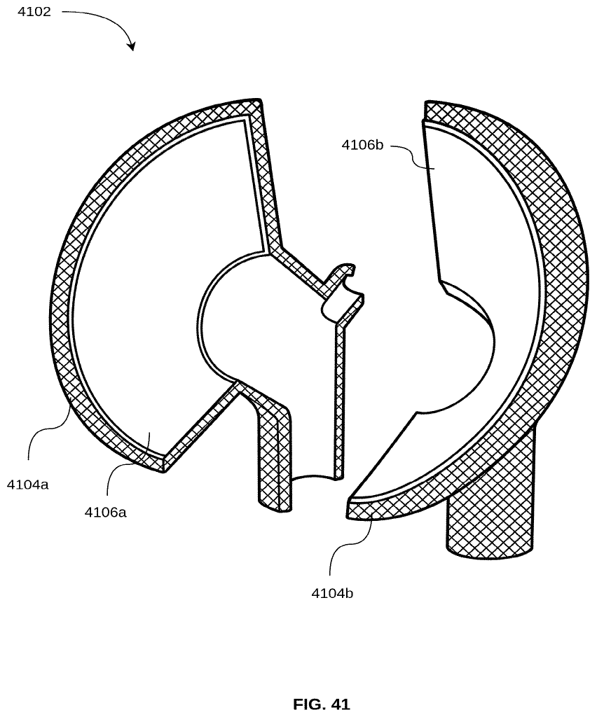

FIG. 41 illustrates an exploded perspective view of a modular breast shield 4102 formed from a laminate material comprising a compressed fiber material layer (e.g., 3804) comprising a full outer surface of the breast pump flange. In this embodiment, the compressed fiber material forms a continuous structural shell across the entire exterior of the breast shield 4102, providing maximum structural rigidity while the smooth inner surfaces of both halves are defined by a barrier layer (e.g., 3806). The compressed fiber material defines a majority of the structural thickness of the component and the barrier layer defines a minority thickness of the component. Structural rigidity of the component is provided primarily by the compressed fiber material rather than by the barrier layer, and the barrier layer has a thickness insufficient to maintain the structural shape of the component in the absence of the compressed fiber material.

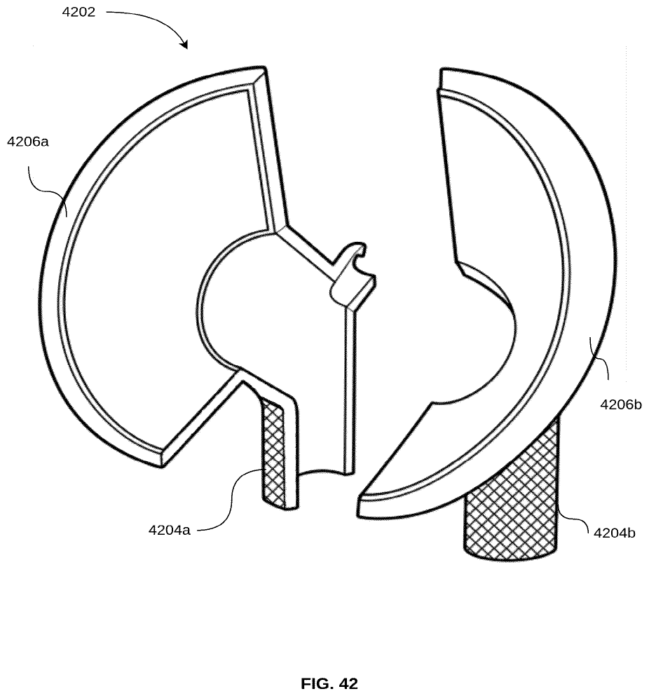

FIG. 42 illustrates an exploded perspective view of a modular breast shield 4202 formed from a laminate material with a barrier layer (e.g., 3806) wrapping over skin-contacting and coupling edges of a compressed fiber material (e.g., 3804). In this embodiment, the barrier layer extends beyond the inner funnel surface to wrap over the outer skin-contacting edges and the coupling edges where the two halves mate together. This configuration ensures that all surfaces that contact skin or that form the mating interface between the two modular halves are covered by the barrier, providing a hygienic, liquid-impermeable, and mechanically compliant surface. The barrier layer may extend across one or more mating surfaces of the first portion and the second portion, and the barrier layers of the first and second portions may be bonded together along the one or more mating surfaces to form a continuous liquid-impermeable barrier across a joint between the first portion and the second portion of the modular breast shield 4202.

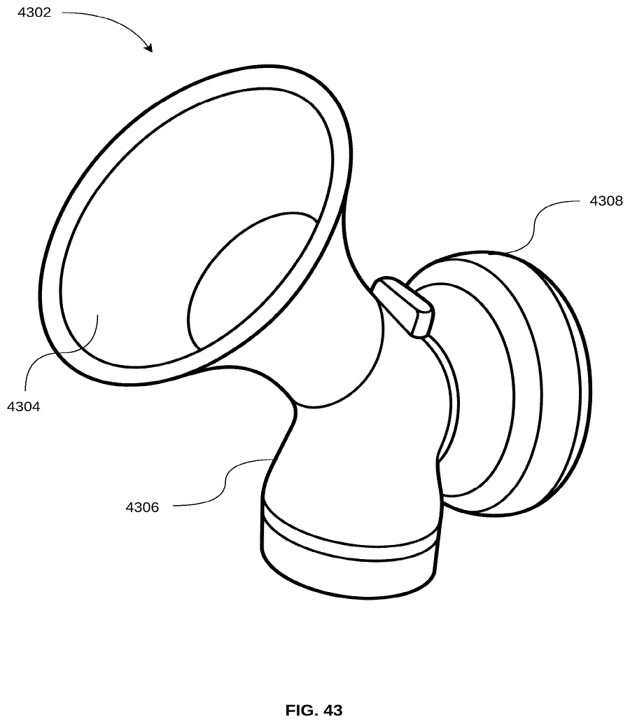

FIG. 43 illustrates a perspective view of a breast pump component 4302 formed from a laminate material, such as 3802. The breast pump component 4302 comprises a funnel-shaped breast shield 4304, flange, body portion, and backflow protector in their assembled configuration.

The laminate material exhibits liquid impermeability and mechanical durability sufficient for use in a breast pump device under partial vacuum conditions. The laminate material resists measurable milk transport into the compressed fiber material during use and maintains barrier integrity during repeated vacuum cycles representative of breast pump operation. The laminate material may be configured to withstand repeated partial-vacuum loading cycles between 80 mmHg and 280 mmHg for at least 1,000 cycles without delamination of the barrier layer from the compressed fiber material and without formation of cracks, creases, or fluid leakage paths.

The laminate material resists collapse and permanent deformation when subjected to partial vacuum conditions during breast pump operation. The laminate material elastically deforms under vacuum pressure and substantially returns to an original shape upon release of the vacuum. The barrier layer is configured to elastically deform under partial vacuum while the compressed fiber material resists permanent deformation. The laminate material is configured to bend or flex under vacuum loading without forming creases or stress concentrations within the compressed fiber material, and the barrier layer distributes vacuum-induced stress across the compressed fiber material to prevent localized failure points.

The laminate material (e.g., 3802) resists structural collapse under a partial vacuum of at least 150 mmHg, at least 200 mmHg, or at least 250 mmHg without permanent deformation. The laminate material maintains dimensional integrity when subjected to repeated vacuum cycles within a range of 50 mmHg to 300 mmHg and exhibits a maximum permanent deformation of less than 5%, less than 3%, or less than 1% after vacuum loading representative of breast pump operation. The laminate material may have a flexural modulus in a range of 0.1 to 5 GPa, 0.3 to 3 GPa, or 0.5 to 2 GPa, measured in bending.

The laminate material (e.g., 3802) can comprise a barrier layer (e.g., 3804) and a compressed fiber material layer (e.g., 3806). The barrier layer may increase the flexural endurance of the laminate material by at least 25%, at least 50%, or at least 100% relative to the compressed fiber material alone. The laminate material withstands at least 500, at least 1,000, or at least 5,000 flexural deformation cycles without cracking or delaminating. The laminate material exhibits an elastic strain at break in a range of 1% to 20%, 2% to 15%, or 5% to 10%. The barrier layer exhibits greater elongation at break than the compressed fiber material and accommodates elastic deformation during vacuum cycling. The laminate material exhibits fatigue resistance sufficient to withstand at least 2,000 vacuum cycles without loss of barrier or mechanical function.

The laminate material (e.g., 3802) exhibits a liquid permeation rate of less than 1 g/m²/day, less than 0.5 g/m²/day, or less than 0.1 g/m²/day. Transport of lactation fluid through the laminate material is substantially prevented over a continuous exposure period of at least 4 hours, at least 12 hours, or at least 24 hours. The barrier layer (e.g., 3806) exhibits a water vapor transmission rate (WVTR) of less than 20 g/m²/day, less than 10 g/m²/day, or less than 5 g/m²/day. The compressed fiber material (e.g., 3804) remains substantially dry after repeated exposure to lactation fluid due to the barrier properties of the barrier layer.

Adhesion between the barrier layer (e.g., 3806) and the compressed fiber material (e.g., 3804) exhibits a peel strength of at least 1 N/cm, at least 3 N/cm, or at least 5 N/cm. The laminate material resists delamination under cyclic mechanical loading representative of breast pump use. Bonding between the barrier layer and the compressed fiber material is maintained after exposure to moisture, vacuum, and mechanical deformation. The laminate material exhibits improved flexural endurance relative to the compressed fiber material alone and demonstrates greater durability under wet conditions than the compressed fiber material alone.

In some embodiments, the barrier layer (e.g., 3806) includes a patterned reinforcement structure selected from lattice patterns, rib patterns, corrugations, or anisotropic reinforcement features configured to increase stiffness in selected regions of the component. The patterned reinforcement may span an interface between the first portion and the second portion to reduce deformation at a seam under vacuum loading. These patterned thin-film reinforcement layers may be laminated, fused, or co-formed onto or within the flange structure to overcome the inherent mechanical limitations of biodegradable materials while maintaining environmental sustainability.

The barrier layer (e.g., 3806) may be configured to satisfy biocompatibility requirements for materials contacting lactation fluid in a medical device pursuant to a biological evaluation conducted in accordance with ISO 10993-1. Materials defining milk-contacting surfaces exhibit no cytotoxic response when evaluated using an in-vitro cytotoxicity assay consistent with ISO 10993-5 and exhibit no irritation or sensitization response when evaluated under conditions consistent with ISO 10993-10. Chemical characterization testing demonstrates that extractable or leachable substances from the barrier layer remain below toxicological concern thresholds established under FDA biocompatibility guidance.

The barrier layer (e.g., 3806) composition may be selected as a food-contact substance authorized for contact with human milk under 21 CFR Parts 175, 176, or 177. The laminate material exhibits overall migration values below 10 mg/dm² when tested under food-contact conditions representative of human milk storage. Specific migration of constituent substances from the barrier layer into human milk remains below FDA-recognized thresholds of toxicological concern. The laminate material maintains food-contact compliance under conditions of refrigerated storage and frozen storage of human milk.

In some embodiments, the compressed fiber material (e.g., 3804) is not suitable for direct contact with lactation fluid, and the barrier layer isolates the compressed fiber material from the lactation fluid. The compressed fiber material may not be approved for direct contact with lactation fluid, and the barrier layer provides a regulatory barrier between the compressed fiber material and the lactation fluid. In some embodiments, the barrier layer (e.g., 3806) is selected to satisfy food-contact or medical-device fluid-contact requirements independently of the compressed fiber material. The compressed fiber material may be selected without regard to food-contact regulatory approval and the barrier layer compensates for such regulatory limitations.

In some embodiments, the laminate architecture enables use of a compressed fiber substrate (e.g., 3804) that is not independently approved for food contact by providing a compliant polymeric barrier (e.g., 3806). The laminate material maintains mechanical integrity under vacuum levels representative of FDA-cleared breast pump operation. Cyclic vacuum testing demonstrates no cracking, delamination, or barrier failure after a number of cycles representative of normal breast pump use. The same laminate material architecture supports compliance with both FDA medical-device requirements for milk expression and FDA food-contact requirements for milk storage.

In some embodiments, the first portion and the second portion may be configured to be shipped in an unassembled state and joined together by a user prior to first use. Assembly of the first and second portions by a user enables formation of an internal fluid-contact surface that does not exist prior to assembly. In some embodiments, the internal fluid-contact surface is completed upon joining of the barrier layers of the first and second portions. In some embodiments, the barrier layer (e.g., 3806) of the first portion may be bonded directly to the barrier layer of the second portion to form a sealed joint. In some embodiments, the compressed fiber materials (e.g., 3804) of the first and second portions are not directly bonded to one another at a fluid-contact interface, and the barrier layer extends continuously across a joint between the first portion and the second portion.

In some embodiments, the breast pump component may comprise a breast pump flange, a flange connector, a valve, a duckbill valve, a backflow preventer, or a lactation fluid collection container. In some embodiments, the component is configured to be biodegradable or compostable after use while maintaining regulatory compliance during use. In some embodiments, neither the first portion nor the second portion is injection molded as a unitary plastic body. In some embodiments, at least one portion of the component has a mass lower than an equivalent injection-molded plastic component of the same geometry, and the compressed fiber material provides a reduction in shipping weight relative to an injection-molded plastic breast pump component providing equivalent structural strength.

In some embodiments, a compostable breast milk storage container is provided comprising two compression-molded fiber halves joined together to define an internal milk storage volume. In some embodiments, each half includes a polymeric biopolymer barrier coating on an inner surface thereof. The two molded fiber halves may be joined by direct heat-sealing of opposing biopolymer barrier layers along a perimeter rim. The storage container may have a top edge wider than a bottom edge such that the container exhibits a trapezoidal cross-section. In some embodiments, the trapezoidal geometry is configured so that a plurality of identical containers stacked in alternating vertical orientation form a level, stable stack. In some embodiments, the geometry provides substantially uniform face-to-face contact between adjacent containers to improve heat transfer during freezing.

The compostable breast milk storage container may further comprise at least one pre-formed port opening disposed along an upper edge of the container and configured to accept a breast pump adapter. In some embodiments, the port opening establishes a sealed fluid pathway from a breast pump directly into the container without an intermediate collection vessel. The port opening may be recessed relative to an outer surface of the container such that port openings of adjacent containers do not mechanically interfere when the containers are stacked in alternating orientations.

A breast milk expression and storage system may be provided comprising a reusable breast pump, a disposable breast pump flange formed from molded fiber with a biopolymer barrier layer defining milk-contacting surfaces, and a disposable breast milk storage container formed from molded fiber with a biopolymer barrier layer. In some embodiments, the system is configured to establish a sealed milk flow path from the breast pump directly into the storage container such that all milk-contacting components are disposable and compostable. In some embodiments, the disposable breast pump flange and the disposable breast milk storage container may be fluidically coupled by a removable adapter configured to interface with multiple breast pump models.

In some embodiments, a breast pump flange is provided comprising a body formed from biodegradable materials and configured to transition between a substantially planar configuration for packaging and a three-dimensional conical configuration for use. In some embodiments, the flange includes predetermined fold lines and reinforcement features that cause the flange to assume the three-dimensional configuration upon attachment to a breast pump or application of vacuum. In some embodiments, the flange may be formed from molded fiber with a biopolymer barrier layer defining milk-contacting surfaces, and elastic energy stored in the flange body may bias the flange toward the three-dimensional configuration.

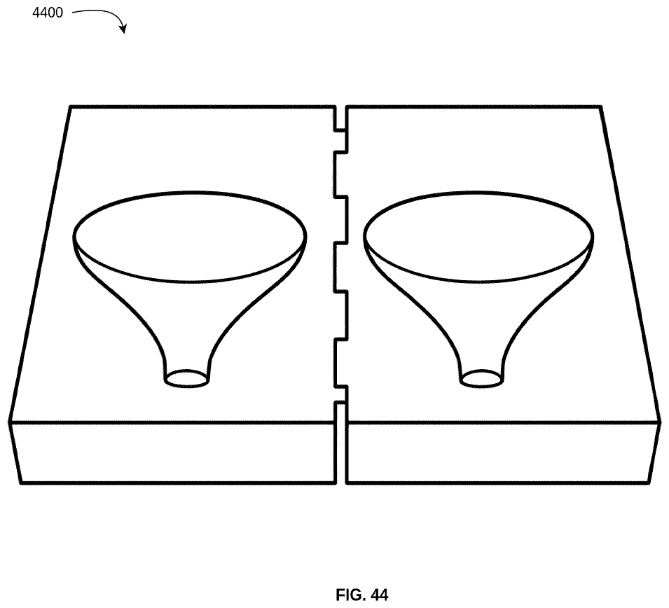

Referring now to FIGS. 44-48, various embodiments of manufacturing methods and apparatus for forming breast pump components from laminate materials are illustrated and described.

FIG. 44 illustrates a perspective view of a thermoforming mold 4400 comprising two symmetrical mold block halves, according to one or more embodiments of the present disclosure. Each mold block half includes a cavity having a funnel-shaped upper region that tapers to a narrower throat section and terminates in a channel at the bottom. The mold cavities are configured to thermoform breast pump component halves from a laminate material comprising a compressed fiber material and a barrier layer. The mating faces of the two mold block halves include interlocking alignment features for registering the halves together during molding operations. The thermoforming mold 4400 may be used to form breast pump flanges, flange connectors, valve housings, or other breast pump components from the laminate material.

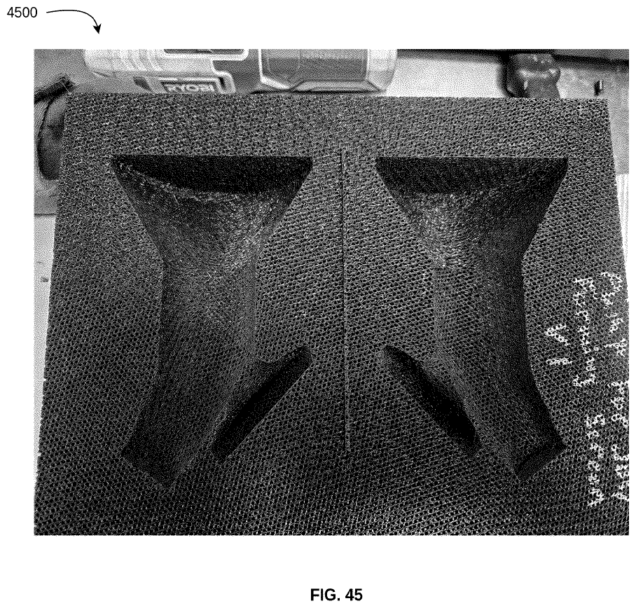

FIG. 45 illustrates a photograph of a forming screen 4500 comprising two symmetrical cavities, according to one or more embodiments of the present disclosure. Each cavity includes a funnel-shaped region corresponding to the breast shield portion of a breast pump component and a narrower section corresponding to the connector base. The forming screen 4500 is configured for use in forming breast pump component halves from a laminate material comprising a compressed fiber material and a barrier layer. The forming screen 4500 may be used in wet molded fiber forming, dry molded fiber forming, or other fiber forming processes to create the compressed fiber material substrate prior to or concurrent with application of the barrier layer.

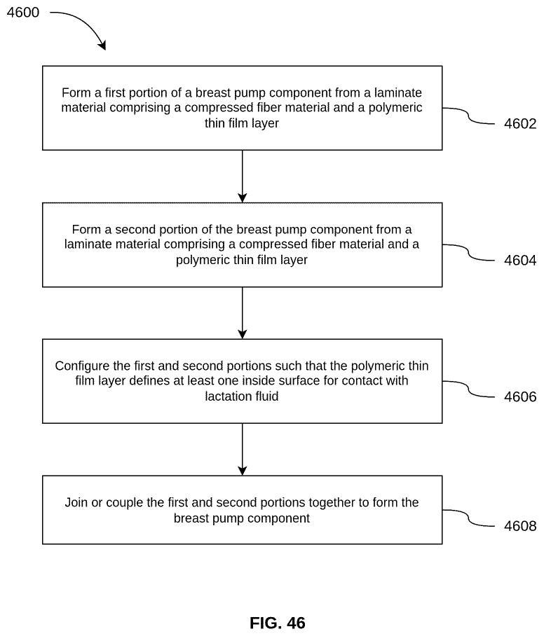

FIG. 46 illustrates a block flow diagram of a method 4600 for forming a breast pump component, according to one or more embodiments of the present disclosure. The method 4600 includes a step 4602 of forming a first portion of a breast pump component from a laminate material comprising a planar compressed fiber material and a barrier layer disposed on at least one surface of the compressed fiber material. The method 4600 includes a step 4604 of forming a second portion of the breast pump component from a laminate material comprising a planar compressed fiber material and a barrier layer disposed on at least one surface of the compressed fiber material. The method 4600 includes a step 4606 of configuring the first portion and the second portion such that the barrier layer of each portion defines at least one inside surface configured for contact with lactation fluid when the breast pump component is assembled. The method 4600 includes a step 4608 of joining or coupling the first portion and the second portion together to form the breast pump component. The compressed fiber material provides structural rigidity, reduced weight, and biodegradability, while the barrier layer provides a liquid-impermeable, food-contact-compatible, and mechanically compliant surface suitable for use in a breast pump device.

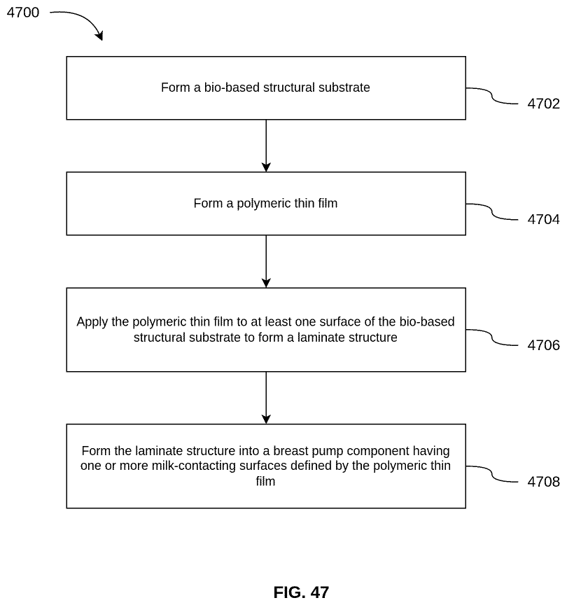

FIG. 47 illustrates a block flow diagram of a method 4700 of manufacturing a breast pump component, according to one or more embodiments of the present disclosure. The method 4700 includes a step 4702 of forming a bio-based structural substrate. The method 4700 includes a step 4704 of forming a barrier. The method 4700 includes a step 4706 of applying the barrier to at least one surface of the bio-based structural substrate to form a laminate structure. The method 4700 includes a step 4708 of forming the laminate structure into a breast pump component having one or more milk-contacting surfaces defined by the barrier. Forming the bio-based structural substrate may comprise wet molded fiber forming, dry molded fiber forming, thermoforming, compression molding, or hot pressing of fibrous material. Forming the barrier may comprise extrusion, blown-film extrusion, cast-film extrusion, solution casting, melt casting, calendaring, or extrusion coating. Applying the barrier to the bio-based structural substrate may comprise heat and pressure lamination, adhesive lamination, extrusion coating directly onto the substrate, spray coating, dip coating in a polymeric dispersion or solution, or roll-to-roll coating or lamination processes.

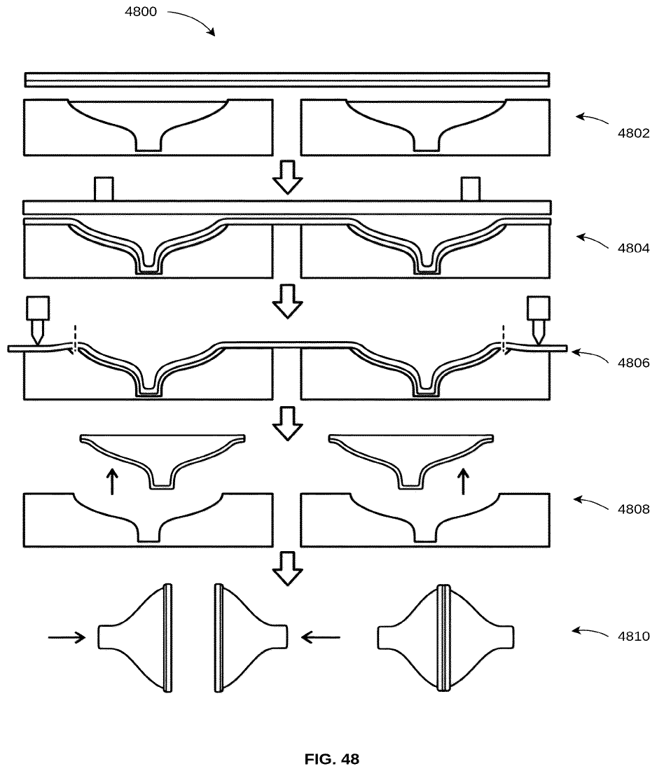

FIG. 48 illustrates a side-view schematic illustration of a thermoforming process 4800 for manufacturing breast pump component halves from a laminate material, according to one or more embodiments of the present disclosure. In a first step 4802, a planar laminate material comprising a compressed fiber material and a barrier layer is positioned over a first breast pump half mold and a second breast pump half mold. In a second step 4804, the laminate material is thermoformed into the mold cavities by press plates to conform to the shape of the breast pump component halves. In a third step 4806, cutting tools trim excess laminate material around the outer edges of the thermoformed halves. In a fourth step 4808, the thermoformed breast pump component halves are released from the molds. In a fifth step 4810, the two breast pump component halves are coupled together to form the breast pump component. The thermoforming process 4800 enables efficient manufacturing of modular breast pump components from the laminate material while maintaining the layered structure of the compressed fiber material and barrier layer throughout the forming process.

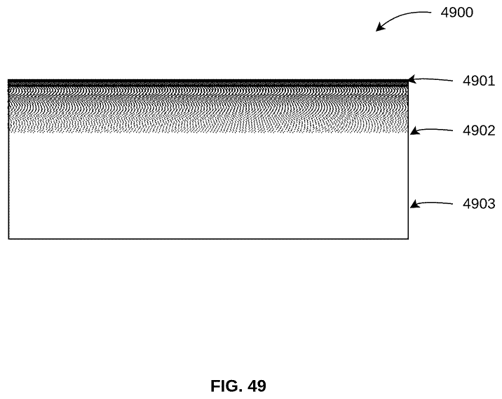

FIG. 49 is a cross-sectional view of a material 4900 comprising a fiber material 4903 i which a barrier material gradient extends from a contact surface 4901 to a barrier material interface 4902 where the barrier material is no longer present in the fiber material 4903. The contact surface 4901 can be impermeable, mostly impermeable, selectively impermeable, and/or substantially impermeable to lactation fluids such as breastmilk. The barrier material can be a liquid in which the fiber material 4903 is dipped or soaked starting at a top surface of the fiber material 4903, wherein the extent to which the barrier material soaks into the fiber material 4903 results in the gradient of barrier material within the fiber material 4903 and/or the distance between the contact surface 4901 and the barrier material interface 4902. Alternatively, the barrier material can be sprayed or otherwise disposed onto an inside surface of the fiber material 4903, and the gradient can naturally occur due to capillarity within pore spaces or inter-fiber spaces within the fiber material 4903.

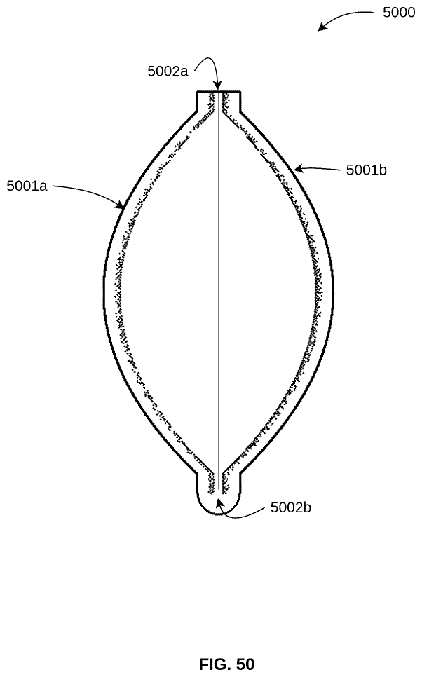

FIG. 50 is a top-down cross-sectional view of a lactation fluid pouch 5000 formed from a material, such the material 4900. The lactation fluid pouch 5000 can comprise a first half 5001a and a second half 5001b formed from a fiber-based material that is coated with a barrier coating, for example. The first and second halves 5001a, 5001b can be formed from one sheet of the material (e.g., 4900) that is bent about a bending point 5002b and coupled together at a coupling point 5002a.

In some embodiments, the method of manufacturing a breast pump component further comprises forming the breast pump component in at least two separate portions and joining the at least two separate portions together to form the breast pump component. Joining the at least two separate portions may comprise heat sealing opposing barrier surfaces to one another, ultrasonic welding, adhesive bonding, mechanical fastening, or combinations thereof. Joining the at least two separate portions may form a liquid-impermeable seam without use of a separate gasket or sealing member.

In some embodiments, a method of manufacturing a user-customized breast pump component is provided comprising selecting one or more target mechanical properties and one or more target geometric dimensions associated with a specific user, selecting material compositions, material layer configurations, and manufacturing parameters based on the selected target mechanical properties and target geometric dimensions, and forming the breast pump component using the selected material compositions, material layer configurations, and manufacturing parameters to achieve the target mechanical properties and target geometric dimensions. The target mechanical properties may comprise one or more of elasticity, flexural modulus, tensile strength, fatigue resistance, tear resistance, creep resistance, or vacuum-induced deformation resistance. The target geometric dimensions may comprise one or more of flange diameter, nipple tunnel diameter, nipple tunnel length, flange cone angle, rim thickness, component wall thickness, or overall component curvature.

In some embodiments, alternative methods for forming modular breast pump component portions may be employed. The compressed fiber material may be formed using vacuum forming processes in which a fibrous slurry is deposited onto a porous mold surface and vacuum is applied to draw water through the mold, consolidating the fibers into a shaped substrate. The vacuum-formed fiber substrate may subsequently be dried, pressed, and laminated with the barrier layer. In other embodiments, the compressed fiber material may be formed using pressure forming in which positive air pressure is applied to force the fibrous material into conformance with a mold cavity. Pressure forming may achieve finer surface detail and tighter dimensional tolerances compared to vacuum forming alone.

In some embodiments, the modular breast pump component portions may be formed using matched-die molding in which the laminate material is pressed between complementary male and female mold halves. Matched-die molding may provide precise control over wall thickness and surface finish on both interior and exterior surfaces of the component portions. The matched-die molding process may be performed at elevated temperatures to soften the barrier layer and improve conformance to the mold geometry while maintaining adhesion to the compressed fiber material.

In some embodiments, rotational molding or centrifugal molding techniques may be employed to form breast pump component portions having complex three-dimensional geometries. The laminate material may be positioned within a rotating mold, and centrifugal force may be used to press the material against the mold surface during heating and forming operations.

In some embodiments, the first portion and the second portion of the breast pump component may be coupled together using interference fit connections in which one portion includes a protruding feature dimensioned to be slightly larger than a corresponding receiving feature on the other portion. The interference fit creates a friction-based connection that resists separation during use. The interference fit may be enhanced by surface texturing, knurling, or ribbing on the mating surfaces.

In some embodiments, the first portion and the second portion may be coupled together using tongue-and-groove joints in which one portion includes a longitudinal tongue extending along its mating edge and the other portion includes a corresponding groove configured to receive the tongue. The tongue-and-groove joint provides alignment and resistance to lateral displacement while the barrier layers on the mating surfaces may be heat-sealed or adhesively bonded to form a liquid-impermeable seal.

In some embodiments, the first portion and the second portion may be coupled together using dovetail joints, mortise-and-tenon joints, or other interlocking mechanical features formed in the compressed fiber material. These mechanical interlocking features may provide structural integrity and alignment while the barrier layers are bonded to provide fluid sealing.

In some embodiments, the first portion and the second portion may be coupled together using ultrasonic welding in which high-frequency vibrations are applied to the mating surfaces to generate localized heating and fusion of the barrier layers. Ultrasonic welding may form a strong, liquid-impermeable bond without the need for additional adhesives or fasteners. The ultrasonic welding parameters, including amplitude, frequency, pressure, and weld time, may be selected based on the thickness and composition of the barrier layer to achieve optimal bond strength.

In some embodiments, the first portion and the second portion may be coupled together using laser welding in which a focused laser beam is directed at the interface between the barrier layers to generate localized melting and fusion. Laser welding may provide precise control over the weld location and energy input, enabling formation of narrow, high-strength seams.

In some embodiments, the first portion and the second portion may be coupled together using solvent bonding in which a solvent is applied to the barrier layers at the mating surfaces to temporarily soften the polymer and enable interdiffusion of polymer chains across the interface. Upon evaporation of the solvent, a strong bond is formed between the barrier layers.

In some embodiments, the first portion and the second portion may be coupled together using a combination of mechanical interlocking features and adhesive or thermal bonding. The mechanical interlocking features may provide initial alignment and structural support during the bonding process, while the adhesive or thermal bond provides the liquid-impermeable seal required for breast pump operation.

In some embodiments, a breast milk storage container may be formed from a translucent paper substrate coated with one or more functional coatings to provide barrier properties suitable for storing expressed human breast milk. The translucent paper substrate may comprise a thin, densified cellulosic sheet having sufficient optical clarity to permit visual assessment of milk volume and condition while maintaining structural integrity during handling and storage. The translucent paper may be formed by refining cellulosic fibers to a high degree of fibrillation, which increases fiber-to-fiber bonding and reduces light scattering within the paper matrix. Supercalendering or other densification processes may be applied to further increase translucency by collapsing air voids within the paper structure.

In some embodiments, the translucent paper substrate is coated with a hydrophobic coating configured to prevent water and aqueous components of breast milk from absorbing into or permeating through the paper. The hydrophobic coating may comprise one or more of natural waxes such as carnauba wax, candelilla wax, beeswax, or soy wax; plant-derived resins; silicone-based coatings; fluorine-free water repellents; alkyl ketene dimer (AKD); alkenyl succinic anhydride (ASA); or biodegradable hydrophobic polymers such as polylactic acid or polyhydroxyalkanoates. The hydrophobic coating may be applied by spray coating, dip coating, rod coating, blade coating, gravure coating, flexographic coating, or slot-die coating processes.

In some embodiments, the translucent paper substrate is additionally or alternatively coated with a lipophobic coating configured to prevent fats and lipids present in breast milk from absorbing into or staining the paper substrate. The lipophobic coating may comprise fluorine-free oil-resistant agents, modified starches, chitosan-based coatings, or other bio-based lipophobic materials. In some embodiments, a single coating formulation provides both hydrophobic and lipophobic properties, eliminating the need for separate coating applications.

In some embodiments, the coated translucent paper may be formed into a breast milk storage bag by folding, creasing, and sealing operations. Two sheets of coated translucent paper may be positioned with their coated surfaces facing inward to define the milk-contacting interior of the bag. The sheets may be sealed along peripheral edges using heat sealing, ultrasonic sealing, adhesive bonding, or mechanical crimping to form a liquid-tight enclosure. The sealing process may be configured to bond the hydrophobic or lipophobic coatings to one another without compromising barrier integrity at the seal locations.

In some embodiments, the breast milk storage bag may be formed from a single sheet of coated translucent paper that is folded along one or more fold lines and sealed along remaining open edges. The fold lines may be pre-scored or embossed into the paper to facilitate consistent folding during bag formation. Gusseted side panels may be incorporated to increase the volumetric capacity of the bag while permitting flat storage when empty.

In some embodiments, the breast milk storage bag includes a resealable closure mechanism at an upper opening configured to permit filling of the bag with expressed milk and subsequent sealing for storage. The resealable closure may comprise a press-to-seal zipper formed from biodegradable polymers, an adhesive strip with a peel-away release liner, a fold-over flap with an integrated adhesive tab, or a mechanical clip or clamp. The closure mechanism may be configured to maintain a liquid-tight seal during refrigerated or frozen storage of expressed breast milk.

In some embodiments, the breast milk storage bag includes graduated volume markings printed or embossed on an exterior surface of the translucent paper to permit measurement of milk volume. The translucency of the paper substrate permits visual observation of the milk level within the bag for comparison against the graduated markings. The volume markings may be printed using food-safe inks that do not migrate through the paper substrate to contact the stored milk.

In some embodiments, the breast milk storage bag includes a pour spout or dispensing feature configured to facilitate controlled pouring of milk from the bag during feeding preparation. The pour spout may be formed by shaping a corner region of the bag into a tapered channel or by incorporating a separate spout component sealed into an opening in the bag.

In some embodiments, the coated translucent paper and the breast milk storage bag formed therefrom are configured to be biodegradable, compostable, or recyclable after use, providing an environmentally sustainable alternative to conventional plastic breast milk storage bags. The hydrophobic and lipophobic coatings may be selected from materials that do not interfere with composting or recycling processes applicable to paper products.

In some embodiments, breast milk storage bags and other milk-contacting components may be formed from alternative bio-based materials. Regenerated cellulose films, such as cellophane, may be employed as a substrate material providing transparency, flexibility, and biodegradability. The regenerated cellulose film may be coated with a moisture barrier layer comprising nitrocellulose, polyvinylidene chloride, or biodegradable alternatives such as shellac or zein protein coatings derived from corn.

In some embodiments, milk-contacting components may be formed from bacterial cellulose, which is produced by microbial fermentation and exhibits high purity, mechanical strength, and water-holding capacity. Bacterial cellulose may be dried and densified to form thin, flexible sheets suitable for forming storage bags or liner components. The bacterial cellulose may be surface-treated with hydrophobic agents to improve moisture resistance while maintaining biocompatibility.

In some embodiments, milk-contacting components may be formed from alginate-based films derived from brown seaweed. Alginate films may be crosslinked with calcium ions to improve mechanical strength and water resistance. The alginate films may be blended with other biopolymers such as pectin, carrageenan, or chitosan to tailor barrier properties and mechanical performance for breast milk storage applications.

In some embodiments, milk-contacting components may be formed from starch-based films derived from corn, potato, tapioca, or other plant sources. The starch films may be plasticized with glycerol, sorbitol, or other food-safe plasticizers to improve flexibility and reduce brittleness. Starch-based films may be blended with polyvinyl alcohol or other water-soluble polymers to enhance film-forming properties and moisture resistance.

In some embodiments, milk-contacting components may be formed from protein-based films derived from casein, whey protein, soy protein, wheat gluten, or collagen. Protein films may provide oxygen barrier properties and may be crosslinked using enzymatic, chemical, or physical methods to improve water resistance and mechanical strength. The protein films may be laminated with other biodegradable materials to form composite structures having enhanced barrier and mechanical properties.

In some embodiments, milk-contacting components may be formed from chitin or chitosan derived from crustacean shells or fungal sources. Chitosan films exhibit antimicrobial properties that may help preserve expressed breast milk during storage. The chitosan films may be blended with other biopolymers or coated with hydrophobic layers to improve moisture barrier performance.

In some embodiments, milk-contacting components may be formed from pullulan, a polysaccharide produced by fungal fermentation. Pullulan films are transparent, flexible, and exhibit excellent oxygen barrier properties. The pullulan films may be coated or laminated with moisture barrier layers to provide comprehensive protection for stored breast milk.

In some embodiments, milk-contacting components may be formed from polyhydroxyalkanoate (PHA) films produced by bacterial fermentation of renewable feedstocks. PHA films are biodegradable and compostable while providing mechanical properties comparable to conventional petroleum-based plastics. Different PHA copolymer compositions may be selected to tailor flexibility, barrier properties, and degradation rates for specific breast pump component applications.

In some embodiments, milk-contacting components may be formed from polylactic acid (PLA) films derived from fermented plant sugars. PLA films may be modified with plasticizers or blended with other biodegradable polymers to improve flexibility and impact resistance. The PLA films may be coated with barrier layers to reduce moisture and oxygen permeation during breast milk storage.

In some embodiments, milk-contacting components may be formed from polybutylene succinate (PBS) or polybutylene adipate terephthalate (PBAT) films, which are biodegradable polyesters exhibiting flexibility and toughness. These materials may be blended with PLA or other biopolymers to achieve desired mechanical and barrier properties for breast pump applications.

In some embodiments, the breast pump system may include one or more pump compatibility adapters configured to interface with breast pumps from specific manufacturers. The pump compatibility adapters may be configured for use with a variety of different breast pump systems and devices. Each pump compatibility adapter may be dimensioned and configured to establish a sealed pneumatic connection between the disposable breast pump components described herein and the vacuum port or connector geometry of a specific pump brand. The pump compatibility adapters may enable cross-brand compatibility, allowing a single set of disposable components to be used with multiple different pump systems through selection of an appropriate adapter.

In some embodiments, the breast pump system may include one or more bag-direct pumping adapters configured to establish a sealed fluid pathway from the breast pump flange directly into a breast milk storage bag without an intermediate collection bottle or container. The bag-direct pumping adapter may comprise a rigid or semi-rigid coupling portion configured to interface with the pump-side portion of the breast shield or flange connector, and a flexible or deformable sealing portion configured to interface with an opening of a breast milk storage bag. The bag-direct pumping adapter may enable expressed milk to flow directly from the breast shield into the storage bag during pumping, eliminating the need to transfer milk from a collection bottle to a storage bag after pumping.

In some embodiments, the barrier layer or biopolymer barrier coating may be applied to the compressed fiber material or molded fiber substrate using aqueous dispersion coating methods. Aqueous dispersion coating may comprise applying an aqueous dispersion or emulsion of biodegradable polymer particles, such as PHA or PLA particles suspended in water, to the fiber substrate surface. The aqueous dispersion may be applied by spray coating, dip coating, rod coating, blade coating, gravure coating, flexographic coating, slot-die coating, curtain coating, or other coating methods suitable for aqueous dispersions. Following application, the coated substrate may be dried to remove water and form a continuous polymeric barrier layer on the fiber surface. Aqueous dispersion coating may provide advantages including reduced use of organic solvents, improved coating uniformity, and compatibility with existing paper coating equipment.

In some embodiments, a vacuum-adaptive biopolymer diaphragm may be provided as a disposable, compostable membrane configured to replace silicone diaphragms commonly used in breast pump systems. The vacuum-adaptive diaphragm may be formed from biodegradable elastomeric materials including, but not limited to, polyhydroxyalkanoates (PHAs), thermoplastic elastomeric starch blends, biodegradable polyurethane analogs, or PLA-based elastomers modified with citrate plasticizers or rubber-like copolymers. The vacuum-adaptive diaphragm may be manufactured through thermoforming, thin-film casting, blow-molding, dip-molding, extrusion-film stretching, or solution casting processes. Additives such as nanocellulose reinforcements, chitosan microfibers, or biodegradable elastomer microparticles may be incorporated into the diaphragm material to increase elasticity or tune stiffness gradients.

In some embodiments, the vacuum-adaptive diaphragm may exhibit vacuum-adaptive behavior arising from non-uniform thickness, variable crystallinity, engineered viscoelasticity, or multi-layer composite effects built into the diaphragm structure. The diaphragm may incorporate a central thin zone that flexes readily under low vacuum, surrounded by an outer ring with greater thickness or higher crystallinity level. When the pump initiates suction, the thin central region may collapse inward with minimal force, reducing startup noise and improving responsiveness. As vacuum increases, the outer ring, which may be engineered to resist deformation more strongly, may stretch less, creating a natural stiffness increase that stabilizes the diaphragm and prevents over-deflection.

In some embodiments, the vacuum-adaptive diaphragm may be formed from a multi-layer composite film where each layer exhibits different viscoelastic properties. A top layer may consist of a high-elasticity biopolymer that deforms easily, while a bottom layer may contain a stiffer, more crystalline polymer or a fiber-reinforced biofilm. During low pressures, only the top layer may engage, producing a soft, compliant response. During higher pressures, the lower layer may begin to bear load, effectively changing the diaphragm's stiffness as the vacuum level rises. The interface between layers may be engineered with micro-textured bonding regions that allow limited shear or slip, further tuning mechanical response.

In some embodiments, vacuum adaptation in the diaphragm may be achieved through strategic patterning. Laser-scored or die-cut micro-grooves may be introduced into specific regions of the diaphragm, reducing stiffness locally. As vacuum increases, these grooves may close, causing stiffness to increase. Corrugated or ribbed geometries may be molded into the diaphragm; these ribs may flatten under initial suction to allow movement but may interlock or stiffen when pulled past a threshold displacement. This may enable a single piece of biodegradable material to emulate the nonlinear stress-strain behavior of high-performance elastomers.

In some embodiments, the vacuum-adaptive diaphragm may be designed with non-uniform moisture absorption or plasticizer distribution such that different regions respond differently to humidity or temperature conditions encountered during pumping. A central region with higher plasticizer concentration may remain softer and more pliable, while drier, denser outer regions may maintain structural boundaries that protect the diaphragm from excessive deformation. As the pumping cycle progresses and the diaphragm warms slightly from repeated flexure, the central region may soften further, producing a self-tuning effect.

In some embodiments, the vacuum-adaptive diaphragm may incorporate phase-changing biopolymer micro-domains that temporarily modify stiffness when exposed to tension. These micro-domains may consist of biodegradable semicrystalline polymers that momentarily reorganize their crystalline structure under strain, effectively locking portions of the diaphragm to resist further stretching. When suction is released, the crystalline structure may relax, returning the diaphragm to a more flexible state. The diaphragm may be formed with variable tension fields, such as concentric tension bands that respond differently to strain, where at low suction levels only inner bands deform and at higher suction levels outer bands engage, limiting further travel and stabilizing pressure waveforms.

In some embodiments, a universal disposable valve cartridge may be provided as a single-use, compostable, self-contained valve-and-milk-path component engineered to interface with a variety of existing breast pump systems regardless of the manufacturer's proprietary connector geometry or suction profile. The universal disposable valve cartridge may be constructed from biodegradable polymers such as PLA, PHA, cellulose-reinforced biocomposites, thermoplastic starch blends, or multilayer biodegradable films. The cartridge structure may incorporate a rigid or semi-rigid outer shell providing dimensional stability, and a flexible, valve-containing inner module fabricated from a thin compostable elastomeric material or laminated film.

In some embodiments, the universal disposable valve cartridge may include a deformable, compressible sealing collar or skirt located at the interface between the cartridge and the pump's vacuum port. The sealing collar may be made from a soft biodegradable elastomer or foamed biopolymer designed to deform to match the geometric variations of multiple pump connectors. When pressed or twisted into place, the collar may compress and conform to the connector's shape, forming an airtight seal without requiring precise mechanical fit. The material behavior may be similar to a gasket or O-ring but may use compostable elastomeric formulations engineered to maintain resilience long enough for a pumping session yet biodegrade efficiently after disposal.

In some embodiments, the internal valve mechanism of the universal disposable valve cartridge may operate using a thin-film flap, micro-slit membrane, or umbrella-style diaphragm formed from a compostable elastomer with controlled thickness and geometry. During pumping, negative pressure generated by the pump may open the valve momentarily, allowing milk to pass forward through the cartridge into the collection container. When suction stops or reverses, the flexible membrane may close rapidly, preventing milk from flowing backward and preventing contamination of upstream pump components.

In some embodiments, the universal disposable valve cartridge may include internal flow channels and pressure-balancing pockets that slow, amplify, or smooth incoming vacuum pulses as needed. These channels may include constrictions, compliant chambers, or tapered pathways that tune the internal pressure response of the cartridge, making it mechanically agnostic to pump-specific pressure signatures. The cartridge may incorporate micro-textured inner surfaces to reduce milk adhesion and facilitate smoother flow through the valve. A multi-layer laminated film valve may be provided where an inner slit is cut through only one layer of the laminate; negative pressure may spread the layers apart at the slit, opening the valve, while positive pressure or milk back-pressure may force the layers to lie flush again, sealing the slit with surface tension and elastic recovery.

In some embodiments, manufacturing of the universal disposable valve cartridge may leverage scalable, low-cost processes such as thin-film thermoforming, ultrasonic welding, heat sealing, and multi-material over-molding. The inner flexible membrane may be cast or extruded as a sheet before being die-cut into shape and inserted into the cartridge housing. The outer housing components may be molded in two halves that capture the valve membrane between them, forming a hermetically sealed module. After pumping, the entire cartridge including all milk-contacting surfaces may be discarded, eliminating the need for cleaning multi-part systems.

In some embodiments, a biodegradable backflow-barrier membrane may be provided as a single-use, compostable valve structure designed to prevent milk from flowing backward into upstream pneumatic pathways of a breast pump. The biodegradable backflow-barrier membrane may be constructed from thin layers of biopolymer films such as polyhydroxyalkanoates (PHAs), biodegradable thermoplastic elastomers derived from renewable feedstocks, PLA-copolymer elastomers modified with citrate or epoxidized oils, or multilayer laminates that combine elastomeric layers with nanocellulose reinforcement. The film thickness may range from 20 to 250 microns depending on mechanical requirements.

In some embodiments, the biodegradable backflow-barrier membrane may comprise a slit-valve micro-membrane where a thin biodegradable film contains a precisely formed micro-slit created by laser scoring or mechanical die-cutting. Under negative pressure from the pump, the slit edges may separate, forming a narrow opening that allows milk to pass through the valve into the collection pathway. When suction is released or when positive pressure develops due to milk attempting to reverse direction, the elastic energy stored in the membrane may cause the slit to reseal instantly. Surface tension from residual milk may further assist the closing mechanism.

In some embodiments, the biodegradable backflow-barrier membrane may comprise a flexible umbrella-style membrane where the film is formed with a domed or conical shape that is anchored at its outer perimeter to the valve seat. Negative pressure may cause the dome to deflect upward and open, while positive pressure may force the dome downward into a sealing ridge or lip. The membrane may also be formed as a hinged flap created by molding or laminating the film so that one region has higher stiffness than the other, allowing predictable bending along a controlled hinge line.

In some embodiments, the biodegradable backflow-barrier membrane may incorporate micro-scale patterning to control its responsiveness. Small embossed textures may reduce stickiness between film surfaces, preventing the slit from adhering shut prematurely. Micro-ribbing or localized thinning may tune deformation so the membrane opens under specific suction thresholds. Anisotropic reinforcement may be achieved by aligning biodegradable micro-fibers or nanocellulose within the film, creating directional stiffness that causes the valve to deform in a prescribed manner. The membrane may be positioned within a compostable valve housing or cartridge, or may be integrated directly into the structure of a disposable flange or milk-handling pathway.

In some embodiments, a fold-flat pop-open flange architecture may be provided comprising a breast pump flange designed to transform between two distinct mechanical states: a folded, substantially planar configuration optimized for sterile packaging, transport, and storage, and a deployed three-dimensional conical configuration suitable for use as a functional breast pump flange. The fold-flat pop-open flange may be constructed from biodegradable, semi-elastic materials such as PLA-based elastomers, PHA blends, thermoplastic starch composites, cellulose-fiber laminates coated with biopolymer films, or thin-wall molded fiber structures treated with moisture-resistant biodegradable coatings.

In some embodiments, the fold-flat pop-open flange may include radial fold lines extending outward from the center toward the outer rim. These fold lines may be created through scoring, reduced-thickness regions, laser-scribed hinge zones, or differential material softening, allowing the flange to fold into an accordion-like or petal-like configuration when packaged. The outer perimeter may include interlocking geometric features such as micro-tabs or compressible bead-edges that hold the flange in its flattened form until the user releases it by bending, twisting, or attaching it to the pump. Additional reinforcing ribs or patterned thin-film laminates may be strategically placed across fold lines to prevent tearing while still allowing reversible deformation.

In some embodiments, the fold-flat pop-open flange may rely on stored elastic energy and engineered shape-memory behavior to pop into its three-dimensional form upon deployment. Reinforced ribs made from a stiffer biodegradable polymer may be pre-curved during manufacturing; when the flange is released from its folded condition, these ribs may naturally return to their curved profile, pushing the flange body toward a conical shape. Differential crystallinity or biaxial stretching of a thin biopolymer film may cause the film to relax toward its original molded curvature when external constraints are removed. Moisture-activated or temperature-responsive relaxation may cause body heat or ambient humidity to soften certain zones of the flange, allowing pre-imposed curvature to emerge.

In some embodiments, the nipple tunnel region of the fold-flat pop-open flange may be manufactured separately or integrally. If integral, the nipple tunnel may be formed as a collapsible cylinder that unfolds into a tubular shape when suction is applied. The pop-open transformation may be further supported by vacuum-assisted unfolding, where negative pressure helps pull the central region forward, straightening fold lines and causing the flange walls to lift into their conical configuration. The geometry of the fold lines may ensure that vacuum preferentially opens the flange rather than causing collapse or buckling.

In some embodiments, the fold-flat pop-open flange architecture may enable roll-to-roll manufacturing, sterile booklet packaging, or dispensing envelopes for efficient storage and distribution. After use, the flange may be easily compacted back into a flat form for disposal or composting, reducing waste volume.

In some embodiments, a dual-density adaptive seal lip may be provided comprising a flange rim made from two biodegradable materials having different mechanical properties. A softer inner band may be configured to conform to breast tissue and prevent leakage, while a stiffer outer support ring may be configured to maintain the geometric shape of the flange rim. Under vacuum, the softer material may mold to the breast surface for a superior seal, while the outer ring may prevent the rim from folding or collapsing. The dual-density behavior may be engineered into the materials and formed in a single molding process.

In some embodiments, a universal vacuum-profile adapter insert may be provided comprising an insert configured to smooth out or amplify suction profiles to make disposable components compatible with different pump brands' pressure curves. The universal vacuum-profile adapter insert may contain internal compliant chambers that expand or compress with each vacuum cycle, altering the waveform of the vacuum before it reaches the flange or valve. This may enable a single disposable component design to perform consistently across multiple pump systems having different native suction characteristics.

In some embodiments, self-stiffening structural ribs may be provided in biopolymer breast pump components. The biopolymer components may include engineered micro-ribs that lock together under compression during suction, increasing stiffness only when needed. During suction, paired ribs may interlock or press into one another, increasing stiffness to resist collapse. When suction is removed, the ribs may disengage, making the component flexible again for comfortable handling and compact storage.

In some embodiments, a leak-resistant compostable flange rim may be provided comprising a flange rim designed with micro-scalloped edges or fluid-locking channels configured to prevent leakage. When pressed against skin, the scallops may increase local pressure at contact points, improving seal quality. Fluid-locking channels may capture micro-amounts of milk or condensation, preventing edge leaks during pumping.

In some embodiments, a collapsible milk pathway with anti-pooling geometry may be provided comprising a milk channel configured to collapse flat when not under suction, preventing milk from settling and spoiling. The pathway may use two thin biodegradable films sealed at the edges; suction may inflate the channel to permit milk flow. When suction stops, the walls may collapse, forcing residual milk forward and downward toward the collection container.

In some embodiments, a disposable multi-size flange with elastic memory may be provided comprising a single flange configured to fit multiple nipple sizes using radial elastic cut patterns. The nipple tunnel may include petal-like slits or flexible sectors that expand or contract under suction, shaping themselves to different nipple diameters. The bio-elastomer material may regain its original size after use, enabling consistent performance across multiple pumping sessions.

In some embodiments, a heat-responsive anatomical fit insert may be provided comprising a milk-contact insert formed from a thermoplastic biodegradable alloy that softens at body temperature. At room temperature, the insert may remain rigid for easy handling and insertion. Once placed against skin, the insert may warm and become supple, forming a custom fit around the nipple and areola for improved comfort and better suction alignment.

In some embodiments, directional flow ribbing for milk transport may be provided comprising disposable milk channels with asymmetric rib geometry configured to guide milk toward the collection container and prevent back-eddies. The ribs may create low-resistance paths that counteract gravitational pooling, improving flow efficiency in compostable pathways that might otherwise have more surface friction than smooth plastics.

In some embodiments, an airflow-optimized compostable tunnel geometry may be provided comprising a nipple tunnel geometry designed to optimize laminar airflow and milk extraction despite being made from softer compostable materials. A multi-diameter, smooth-transition tunnel may reduce turbulence and maintain steady suction even if wall stiffness is lower than conventional plastic equivalents.

In some embodiments, a biodegradable one-way micro-valve may be provided comprising a paper-polymer or starch-polymer micro-valve configured to allow flow in only one direction. The valve may use a hinging flap or micro-slit that opens under forward flow and immediately closes to block reverse flow, eliminating reliance on silicone duckbill valves.

In some embodiments, a disposable comfort-texture inner surface may be provided comprising a textured inner surface configured to reduce nipple friction during suction. The texture may be formed with micro-dimples or wave patterns added during molding, reducing surface area contact, minimizing sticking, and allowing milk film to reduce friction further.

In some embodiments, a universal pump-connector compression collar may be provided comprising a disposable collar made of compressible biodegradable foam-polymer composite configured to fit over output ports of many pump systems. The foam may deform to match the pump's connector geometry and rebound to form a tight seal, allowing compostable flanges or valves to be compatible with different pump models.

In some embodiments, a pre-sterilized compostable diaphragm cap may be provided comprising a cap covering the diaphragm chamber made entirely from biodegradable materials. The cap may include a precision-molded port for the vacuum line and may maintain dimensional stability despite being compostable. The cap may snap into place with compliant locking features.

In some embodiments, an integrated sterile-pack-to-flange deployment system may be provided wherein the sterile wrapper itself becomes part of the flange. The flange may be sandwiched between two compostable films that peel apart during deployment; one layer may stay attached as a shield or structural element. This configuration may avoid contamination, reduce packaging waste, and simplify the user workflow by eliminating separate packaging disposal steps.

In some embodiments, a flexible compostable breast milk storage bag may be provided comprising a flexible fiber-based substrate and a barrier layer disposed on at least one interior surface of the flexible fiber-based substrate. The flexible fiber-based substrate and the barrier layer together may define a sealed or sealable internal volume configured to store expressed human breast milk. The barrier layer may provide liquid impermeability sufficient to contain expressed breast milk under refrigerated and frozen storage conditions. The flexible compostable breast milk storage bag may be compostable without separation of constituent materials.

In some embodiments, the flexible fiber-based substrate may comprise glassine paper. Glassine paper may be a smooth, glossy, translucent or semi-translucent paper formed by supercalendering cellulosic fibers to create a dense, grease-resistant sheet. The glassine paper may have a basis weight in a range of 20 to 80 grams per square meter, 30 to 60 grams per square meter, or 40 to 50 grams per square meter. The glassine paper may exhibit translucency sufficient to permit visual observation of milk volume and condition within the storage bag. The glassine paper may be food-grade glassine approved for direct contact with human milk.

In some embodiments, the barrier layer disposed on the flexible fiber-based substrate may comprise a bio-based barrier coating. The bio-based barrier coating may comprise a plant-derived, heat-sealable barrier material derived from renewable agricultural feedstocks. The bio-based barrier coating may comprise one or more of polyhydroxyalkanoates (PHA), polylactic acid (PLA), polybutylene adipate terephthalate (PBAT), polycaprolactone, starch-based polymers, protein-based polymers, or combinations thereof. In some embodiments, the bio-based barrier coating may be derived from sugarcane-based chemistry. The bio-based barrier coating may be applied to the glassine paper substrate at coating weights sufficient for liquid containment under refrigerated and frozen storage conditions.

In some embodiments, the flexible compostable breast milk storage bag may be plastic-free and PFAS-free. The bag may contain no polyethylene, polypropylene, polyvinyl chloride, or other petroleum-based plastic materials. The bag may contain no per- and polyfluoroalkyl substances (PFAS) or other fluorinated compounds. The flexible compostable breast milk storage bag may be industrially compostable, home compostable, or both. The bag may be certified compostable according to ASTM D6400, ASTM D6868, EN 13432, or other recognized compostability standards.

In some embodiments, the flexible compostable breast milk storage bag may comprise an open-top configuration with a gusseted base configured to provide stand-up capability when the bag is filled with expressed milk. The gusseted base may comprise one or more fold lines or pleats in the fiber-based substrate that expand when the bag is filled, allowing the bag to stand upright on a flat surface. The gusseted base may be formed by folding and sealing the fiber-based substrate along predetermined fold lines during bag manufacturing. The stand-up capability may facilitate filling of the bag during pumping and may improve storage efficiency in refrigerators or freezers.

In some embodiments, the flexible compostable breast milk storage bag may comprise a three-side sealed construction with an open top configured for consumer filling and sealing. Three edges of the bag may be sealed during manufacturing using heat sealing, ultrasonic sealing, or adhesive bonding, while a fourth edge remains open to permit filling. The open top may be configured to receive expressed milk by pouring or by direct connection to a breast pump adapter. After filling, the open top may be sealed by the user using a separate heat sealer, adhesive closure, mechanical clip, or fold-over seal.

In some embodiments, the flexible compostable breast milk storage bag may include a tear notch at a sealed top edge configured for easy consumer opening after sealing. The tear notch may comprise a small cut, perforation, or weakened region in the fiber-based substrate and barrier layer that initiates controlled tearing when the user pulls on the bag. The tear notch may be positioned at a corner or along an edge of the sealed top to facilitate one-handed opening. The tear notch may be formed during bag manufacturing using die cutting, laser cutting, or perforation processes.

In some embodiments, the flexible compostable breast milk storage bag may include volume markings visible through the translucent fiber-based substrate to permit measurement of milk volume without opening the bag. The volume markings may be printed on an exterior surface of the bag using food-safe inks. The volume markings may indicate volumes in milliliters, fluid ounces, or both. The translucency of the glassine paper or other fiber-based substrate may permit visual observation of the milk level within the bag for comparison against the volume markings.

In some embodiments, the flexible compostable breast milk storage bag may be configured for direct pump-to-bag filling. The bag may include an opening or port configured to receive a breast pump adapter or to interface directly with a duckbill valve or other component of a breast pump device, establishing a sealed fluid pathway from the breast pump into the bag without an intermediate collection vessel. The bag-direct pumping adapter described herein may be configured to interface with the opening of the flexible compostable breast milk storage bag. Direct pump-to-bag filling may eliminate the need to transfer expressed milk from a collection bottle to a storage bag after pumping, reducing handling steps and contamination risk.

In some embodiments, the interior of the flexible compostable breast milk storage bag may be sanitized or sterilized prior to final sealing using non-thermal methods. Non-thermal sanitization or sterilization methods may include UV-C irradiation, electron-beam (e-beam) radiation, gamma radiation, or other ionizing or non-ionizing treatment methods. UV-C irradiation may be applied to the interior surfaces of the bag after forming but before final sealing to reduce microbial load. Electron-beam or gamma radiation may be applied to the sealed bag to achieve terminal sterilization. The use of low-dose ionizing radiation treatment may be compliant with 21 CFR 179.45 as applied to paper-based food contact packaging.

In some embodiments, a sequential manufacturing process for the flexible compostable breast milk storage bag may comprise forming the bag from the coated fiber-based substrate, sanitizing or sterilizing the bag interior, heat-sealing the bag closed along one or more edges, and packaging the sealed bag in compostable outer packaging. The bag may be formed by cutting the coated substrate to a predetermined shape and folding along predetermined fold lines. The bag may be sealed along three edges using heat sealing equipment configured for the specific seal parameters of the bio-based coated substrate. The sealed bag may be packaged in a compostable outer pouch or wrapper for distribution.

In some embodiments, a breast milk storage system may be provided comprising the flexible compostable breast milk storage bag and a dedicated consumer heat sealer. The heat sealer may be a purpose-built device optimized for the specific seal parameters of the bio-based coated fiber-based substrate. The heat sealer may include heated jaws or bars configured to apply controlled temperature and pressure to the open edge of the bag to fuse the barrier layers together and form a liquid-tight seal. The heat sealer may include temperature control, pressure control, and dwell time control to ensure consistent seal quality across different bag sizes and substrate thicknesses. The breast milk storage system may be configured as an insurance-billable breast milk storage system under HCPCS code A4287 or other applicable reimbursement codes.

In some embodiments, the flexible compostable breast milk storage bag may be provided as part of a kit with one or more disposable breast pump components described herein. The kit may include one or more flexible compostable breast milk storage bags, one or more pump compatibility adapters, and one or more bag-direct pumping adapters. The kit may enable a complete pump-to-storage workflow using entirely disposable and compostable components. The flexible compostable breast milk storage bag may serve as the lactation fluid collection container in any of the breast pump systems, devices, or kits described herein.

In some embodiments, the flexible compostable breast milk storage bag may be formed from alternative fiber-based substrates including but not limited to regenerated cellulose films, bacterial cellulose sheets, or other densified cellulosic materials coated with one or more of the barrier materials described herein. Regenerated cellulose films such as cellophane may provide transparency and flexibility. Bacterial cellulose sheets may provide high purity and mechanical strength. The barrier layer on the flexible compostable breast milk storage bag may comprise any of the barrier layer materials described herein for use with compressed fiber materials, including PHA, PLA, PBAT, or combinations thereof.

In some embodiments, the flexible compostable breast milk storage bag may exhibit liquid impermeability sufficient to prevent leakage of expressed breast milk during storage, transport, refrigeration, and freezing. The bag may maintain liquid impermeability for at least 24 hours, at least 48 hours, at least 72 hours, or at least one week under refrigerated storage conditions. The bag may maintain liquid impermeability for at least one month, at least three months, or at least six months under frozen storage conditions. The barrier layer may prevent absorption of milk into the fiber-based substrate and may prevent permeation of milk through the bag walls.

In some embodiments, the flexible compostable breast milk storage bag may be configured to withstand freezing and thawing cycles without loss of barrier integrity or mechanical failure. The bag may be filled with expressed milk, sealed, and frozen at temperatures of-20 ° C. or lower. Upon thawing, the bag may maintain its seal integrity and may not exhibit cracking, delamination, or leakage. The bag may withstand at least one freeze-thaw cycle, at least three freeze-thaw cycles, or at least five freeze-thaw cycles without failure.

In some embodiments, the flexible compostable breast milk storage bag may be configured to be compliant with FDA food-contact requirements for materials contacting human milk. The fiber-based substrate and barrier layer may be selected from materials authorized for food contact under 21 CFR Parts 175, 176, or 177. The bag may exhibit overall migration values below 10 mg/dm² when tested under food-contact conditions representative of human milk storage. Specific migration of constituent substances from the barrier layer into human milk may remain below FDA-recognized thresholds of toxicological concern. The bag may be suitable for use under conditions of refrigerated storage and frozen storage of human milk.

Many modifications and other embodiments of the inventions set forth herein will come to mind to one skilled in the art to which these inventions pertain having the benefit of teachings presented in the foregoing descriptions and the associated drawings. Although the figures only show certain components of the apparatus and systems described herein, it is understood that various other components may be used in conjunction with the system. Therefore, it is to be understood that the inventions are not to be limited to the specific embodiments disclosed and that modifications and other embodiments are intended to be included within the scope of the appended claims. Moreover, the steps in the method described above may not necessarily occur in the order depicted in the accompanying diagrams, and in some cases one or more of the steps depicted may occur substantially simultaneously, or additional steps may be involved. Although specific terms are employed herein, they are used in a generic and descriptive sense only and not for purposes of limitation. Specific equipment and materials described in the examples are for illustration only and not for purposes of limitation. For instance, any and all articles, portions of articles, structures, bulk materials, and/or the like, having any form factor, scale, dimensions, aesthetic attributes, material properties, internal structures, and/or mechanical properties, which are formed according to any of the disclosed methods, approaches, processes, or variations thereof, using any devices, equipment, apparatuses, systems, or variations thereof, using any of the build material, printing mixture, ink, yield-stress support material, or other material compositions described herein or variations thereof, are all contemplated and covered by the present disclosure. None of the examples provided are intended to, nor should they, limit in any way the scope of the present disclosure.

A number of implementations have been described. Nevertheless, it will be understood that various modifications may be made without departing from the spirit and scope of the disclosure. Accordingly, other implementations are within the scope of the following claims.

1. A breast pump component comprising:
a first portion of the breast pump component; and
a second portion of the breast pump component,
wherein the first portion and the second portion are configured to be coupled together to form the breast pump component,
wherein each of the first portion and the second portion is formed from a laminate material comprising a compressed fiber material and a barrier layer disposed on at least one surface of the compressed fiber material, and
wherein, in an assembled configuration, the barrier layer of each of the first portion and the second portion defines at least one internal surface of the breast pump component that is configured for contact with lactation fluid.

2. The breast pump component of claim 1, wherein the compressed fiber material provides structural rigidity and reduced weight of the breast pump component and the barrier layer provides liquid impermeability and mechanical compliance.

3. The breast pump component of claim 1, wherein the compressed fiber material is biodegradable or compostable.

4. The breast pump component of claim 1, wherein the barrier layer is disposed over one or more mating surfaces of the first portion and the second portion.

5. The breast pump component of claim 4, wherein the barrier layer forms a continuous liquid-impermeable barrier across a joint between the first portion and the second portion.

6. The breast pump component of claim 1, wherein the compressed fiber material is not suitable for direct contact with lactation fluid and the barrier layer isolates the compressed fiber material from the lactation fluid.

7. The breast pump component of claim 1, wherein the laminate material resists collapse and permanent deformation when subjected to partial vacuum conditions during breast pump operation.

8. The breast pump component of claim 7, wherein the laminate material elastically deforms under vacuum pressure and substantially returns to an original shape upon release of the vacuum.

9. The breast pump component of claim 1, wherein the barrier layer reduces liquid permeation through the laminate material relative to the compressed fiber material alone.

10. The breast pump component of claim 1, wherein the breast pump component comprises a breast pump flange, a valve, a duckbill valve, a flange connector, or a lactation fluid collection container.

11. A breast pump component comprising:
a first portion and a second portion that are coupled together to form the breast pump component,
wherein each of the first portion and the second portion is formed from a laminate material comprising a planar compressed fiber material and a barrier layer laminated onto the compressed fiber material,
wherein the barrier layers of the first portion and the second portion collectively define a continuous internal surface of the breast pump component configured for contact with lactation fluid,
and wherein the laminate material exhibits liquid impermeability and mechanical durability sufficient for use in a breast pump device under partial vacuum conditions.

12. The breast pump component of claim 11, wherein the laminate material resists measurable transport of lactation fluid into the compressed fiber material during use of the breast pump component.

13. The breast pump component of claim 11, wherein the laminate material maintains barrier integrity during repeated vacuum cycles representative of breast pump operation.

14. The breast pump component of claim 11, wherein the barrier layer has a thickness less than a thickness of the compressed fiber material.

15. The breast pump component of claim 11, wherein adhesion between the barrier layer and the compressed fiber material resists delamination during flexural deformation.

16. The breast pump component of claim 11, wherein the laminate material exhibits improved flexural endurance relative to the compressed fiber material alone.

17. The breast pump component of claim 11, wherein the barrier layer is selected to meet food-contact and medical-device milk-contact requirements.

18. The breast pump component of claim 11, wherein the laminate material supports both milk expression in a breast pump device and storage of expressed milk.

19. The breast pump component of claim 11, wherein the compressed fiber material comprises one or more cellulosic fiber materials.

20. A breast pump component comprising:
a first portion and a second portion coupled together to form the breast pump component,
wherein each of the first portion and the second portion is formed from a laminate material comprising a compressed cellulosic fiber material and a barrier layer laminated onto an internal surface of the compressed cellulosic fiber material,
wherein the barrier layer defines all milk-contacting surfaces of the breast pump component,
and wherein the laminate material exhibits both liquid impermeability sufficient to prevent milk absorption into the compressed cellulosic fiber material and elastic flexure sufficient to withstand repeated partial-vacuum pumping cycles without structural failure.

21-305. (canceled)
